(19) United States
(12) Patent Application Publication
(10) Pub. No.: US 2020/0216900 A1
(43) Pub. Date: Jul. 9, 2020

(54) NASAL BIOMARKERS OF ASTHMA

(71) Applicant: Icahn School Of Medicine at Mount Sinai, New York, NY (US)

(72) Inventors: Supinda Bunyavanich, New York, NY (US); Gaurav Pandey, New York, NY (US); Eric S. Schadt, Rye, NY (US)

(73) Assignee: Icahn School Of Medicine at Mount Sinai, New York, NY (US)

(21) Appl. No.: 15/999,796

(22) PCT Filed: Feb. 17, 2017
(86) PCT No.: PCT/US2017/018318

§ 371 Date: Aug. 17, 2018

Related U.S. Application Data
(60) Provisional application No. 62/296,291, filed on Feb. 17, 2016.
(60) Provisional application No. 62/296,915, filed on Feb. 18, 2016.

(51) Int. Cl. C12Q 1/6883; G16B 40/00; G06F 17/18; G16B 50/30; G16B 50/10

(52) U.S. Cl. CPC C12Q 1/6883; G16B 40/00; G06F 17/18; G01N 2800/122; G16B 50/10; C12Q 2600/158; G16B 50/30

(57) ABSTRACT

Asthma is a common, under-diagnosed disease affecting all ages. Mild to moderate asthma is particularly difficult to diagnose given currently available tools. A nasal biomarker of asthma is of high interest given the accessibility of the nose and shared airway biology between the upper and lower respiratory tract. A machine learning pipeline identified an asthma gene panel of 275 unique nasally-expressed genes interpreted via different classification models. This asthma gene panel can be utilized to reliably diagnose asthma in patients, including mild to moderate asthma, in a non-invasive manner and to distinguish asthma from other respiratory disorders, allowing appropriate treatment of the patient's asthma.

### CROSS-REFERENCE TO RELATED APPLICATIONS

This application claims priority to U.S. Provisional Application No. 62/296,291, filed on 17 Feb. 2016 and 62/296,915, filed on 18 Feb. 2016, the disclosures of each of which are herein incorporated by reference in their entirety.

### GOVERNMENT SPONSORSHIP

This invention was made with government support under Grant Nos. R01GM114434, K08AI093538 and R01AI118833, all awarded by the National Institutes of Health (NIH). The government has certain rights in the invention.

### BACKGROUND OF THE INVENTION

### 1. Field of the Invention

Embodiments of the present invention relate generally to methods for diagnosis and monitoring of asthma, including but not limited to mild to moderate asthma, and its differentiation from other respiratory disorders by determining the expression profiles of asthma-specific genes in nasal brushing samples.

### 2. Background

Asthma is a chronic respiratory disease that affects 8.6% of children and 7.4% of adults in the United States¹. The true prevalence of asthma may be higher than these estimates. In one study of US middle school children, 11% reported physician-diagnosed asthma with current symptoms, while an additional 17% reported active asthma-like symptoms without a diagnosis of asthma². Undiagnosed asthma leads to missed school and work, restricted activity, emergency department visits, and hospitalizations^{2, 3}. Mild to moderate asthma in particular can be difficult to diagnose, as it intrinsically involves fluctuating symptoms and signs⁴. The airflow obstruction, bronchial hyper-responsiveness and airway inflammation that characterize asthma are challenging to assess routinely and easily⁴. Given the high prevalence of asthma, there is high potential impact of improved diagnostic tools on reducing morbidity and mortality from asthma. Biomarkers could improve the identification of mild/moderate asthma so that appropriate management can be pursued.

National and international guidelines recommend that the diagnosis of asthma should be based on a history of typical symptoms and objective findings of variable expiratory airflow limitation^{6, 7}. However, obtaining such objective findings is challenging given currently available tools. Pulmonary function tests (PFTs) require equipment, expertise, and experience to execute well^{8, 9}. Many individuals have difficulty with PFTs (e.g., spirometry) because they require coordinated breaths into a device. Results are unreliable if the procedure is done with poor technique⁸. Large epidemiologic studies of both children and adults substantiate that despite guidelines recommending objective tests such as PFTs to assess possible asthma, PFTs are not done in over half of patients suspected of having asthma⁸. Induced sputum and exhaled nitric oxide have been explored as asthma biomarkers, but their implementation requires technical expertise and does not yield better clinical results than physician-guided management alone¹⁰. Given the above, the reality is that most asthma is still clinically diagnosed and managed in children and adults based on self-report^{8, 9}. This is suboptimal for mild/moderate asthma given its waxing/waning nature, and because self-reported symptoms and medication use are biased¹¹. There is need to improve asthma diagnosis, and an accurate biomarker of mild/moderate asthma could help meet that need. The ideal biomarker of mild/moderate asthma would be (1) obtainable noninvasively, (2) obtainable quickly, (3) interpretable without substantial expertise or infrastructure.

A nasal biomarker of asthma is of high interest given the accessibility of the nose and shared airway biology between the upper and lower respiratory tracts^{12, 13, 14, 15}. The easily accessible nasal passages are directly connected to the lungs and exposed to common environmental and microbial factors. An accurate nasal biomarker of asthma that could be quickly obtained by a simple nasal brush could improve asthma diagnosis in adult and pediatric populations.

An asthma-specific gene panel has high potential to be used as a non-invasive biomarker to aid in asthma diagnosis, as it can be quickly obtained by simple nasal brush, does not require machinery for collection, and is easily interpreted. As discussed herein, objective findings of asthma are often not obtainable. Patients with mild/moderate asthma may not be asymptomatic at the time of the clinical encounter, so they may have no detectable wheezing or cough on exam. In many cases, then, a clinician may diagnose asthma on the basis of history alone, and this contributes to the under-diagnosis and misclassification of asthma. Studies have shown that patients with active asthma under-perceive their symptoms and do not tell their primary care physician. An objective diagnostic tool that is easy and quick to obtain and interpret with minimal effort required by the provider and patient could improve asthma diagnosis so that appropriate management can be pursued. A nasal brush-based asthma gene panel meets these biomarker criteria and capitalizes on the common biology of the upper and lower airway, a concept supported by clinical practice and previous findings.

In finding nasal biomarkers of mild/moderate asthma (FIG. 1), the inventors used next-generation RNA sequencing and data analysis to comprehensively profile nasal epithelial gene expression from nasal brushings collected from a well-characterized cohort of subjects with mild/moderate asthma and non-asthmatic controls. These technologies have contributed to advances in several areas of biomedicine, such as disease biomarker identification¹⁶, personalized medicine and treatment¹⁷. Specifically, the inventors used RNA sequencing to comprehensively profile gene expression from nasal brushings collected from subjects with mild to moderate asthma and controls. Using a robust machine learning-based pipeline comprised of feature selection¹⁸, classification¹⁹ and statistical analyses of performance²⁰, the inventors identified a gene panel with 275 unique genes, and subsets specific for different classification analyses, that can accurately differentiate subjects with and without mild-moderate asthma. This asthma gene panel was validated on eight test sets of independent subjects with asthma and other respiratory conditions, finding that it performed with high accuracy, sensitivity, and specificity. As used herein, the term “asthma gene panel” refers to these 275 genes collectively (see Table 4 for the list of genes and subsets). A subset of the asthma gene panel, the LR-RFE & Logistic asthma gene panel, was tested on three additional, independent cohorts of asthmatics and controls, and this panel consistently performed with accuracy. Further testing of the LR-RFE & Logistic asthma gene panel on five cohorts with non-asthma respiratory diseases validated the specificity of this nasal biomarker panel to asthma. The asthma gene panel currently identified through machine learning can be applied as a nasal brush-based biomarker tool for the clinical diagnosis of asthma, including mild/moderate asthma, and for distinguishing asthma from other respiratory disorders. Both diagnosis and differentiation with the invented methods enable the accurate diagnosis and treatment of asthma, including mild to moderate asthma, in the patient.

What is needed, therefore, is a noninvasive, quick and simple method for reliably diagnosing and/or classifying asthma, including but not limited to mild to moderate asthma, as well as distinguishing asthma from other respiratory disorders, and subsequently treating the patient appropriately. It is to such a method that embodiments of the present invention are primarily directed.

### BRIEF SUMMARY OF THE INVENTION

As specified in the Background Section, there is a great need in the art to identify technologies for reliable, consistent, simple and non-invasive diagnosis of asthma, including but not limited to mild to moderate asthma, and use this understanding to develop novel diagnostic methods. The present invention satisfies this and other needs. Embodiments of the present invention relate generally to methods for diagnosis, classification and monitoring of asthma, including but not limited to mild to moderate asthma, and its differentiation from other respiratory disorders by determining the expression profiles of asthma-specific genes in nasal swab/scraping/brushing/wash/sponge samples.

In one aspect, the present invention provides a method for diagnosing asthma in a subject, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In another aspect, the present invention provides a method for detection of asthma in a subject, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In one aspect, the present invention provides a method for differentially diagnosing asthma from other respiratory disorders in a subject, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In one aspect, the present invention provides a method for classifying a subject as having asthma or not having asthma, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In another aspect, the present invention provides a method for monitoring asthma in a subject, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In one aspect, the present invention provides a method for selecting a subject for a clinical trial for asthma therapeutic compositions and/or methods, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold; and

d) identifying the subject as (i) having asthma when the probability output is greater than or equal to the optimal classification threshold or (ii) not having asthma when the probability output is less than the optimal classification threshold.

In one aspect, the present invention provides a method for treating asthma in a subject, comprising the steps of:

a) measuring the gene expression profile(s) of at least one of the genes in the asthma gene panel in a nasal swab/scraping/brushing/wash/sponge collected from the subject;

b) performing classification analysis on the gene counts obtained from the gene expression profile(s);

c) comparing the probability output obtained from the classification analysis to the optimal classification threshold;

d) identifying the subject as (i) having asthma when the probability output is greater than or equal to the optimal classification threshold or (ii) not having asthma when the probability output is less than the optimal classification threshold; and

e) utilizing appropriate therapeutic compositions and/or methods if the subject has asthma.

In one aspect, the present invention provides a kit for diagnosing and/or detecting asthma in a subject, said kit comprising probes directed towards one or more of the genes in the asthma gene panel, as described in more detail herein, wherein the probes can be used to determine the expression levels of one or more of the genes in the asthma gene panel. The kit can also comprise (i) a detection means and/or (ii) an amplification means. The kit may further optionally include control probe sets for detection of control RNA in order to provide a control level as described herein.

In another aspect, the present invention provides a kit for diagnosing and/or detecting asthma in a subject, said kit comprising pairs of oligonucleotides directed towards one or more of the genes in the asthma gene panel, as described in more detail herein, wherein the pairs of oligonucleotides can be used to determine the expression levels of one or more of the genes in the asthma gene panel. The kit can also comprise (i) a detection means and/or (ii) an amplification means. The kit may further optionally include control primer/oligonucleotide sets for detection of control RNA in order to provide a control level as described herein.

In any of the above embodiments, step (a) further comprises the steps of (i) brushing, swabbing, scraping, washing or sponging the patient's nose, (ii) obtaining and appropriately preserving the nasal brushing/swab/scraping/wash/sponge sample, and (iii) assaying the gene expression profile of the cells and tissue contained in the sample, whether by isolating RNA as described herein or by use of a RNA profiling system that does not require a separate isolation step (such as, for example and not limitation, nanoString).

In any of the above embodiments, steps (b) and/or (c) and/or (d) are performed by a computer.

In any of the above embodiments, the classification analysis can comprise the Logistic Regression-Recursive Feature Elimination (LR-RFE) algorithm in combination with the Logistic algorithm as described in more detail below, with the gene expression profiles analyzed by this LR-RFE & Logistic model being the expression profiles of the genes in the LR-RFE & Logistic asthma gene panel. In this embodiment, the optimal classification threshold is about 0.76.

In any of the above embodiments, the classification analysis can alternatively comprise the LR-RFE & SVM-Linear combination model as described in more detail below, with the gene expression profiles analyzed by this model being the expression profiles of the genes in the LR-RFE & SVM-Linear asthma gene panel. The optimal classification threshold for this model is about 0.52.

In any of the above embodiments, the classification analysis can alternatively comprise the SVM-RFE & SVM-Linear model as described in more detail below, the gene expression profiles analyzed by this model being the expression profiles of the genes in the SVM-RFE & SVM-Linear asthma gene panel, and the optimal classification threshold for this model is about 0.64.

In any of the above embodiments, the classification analysis can alternatively comprise the SVM-RFE & Logistic model as described in more detail below, the gene expression profiles analyzed by this model being the expression profiles of the genes in the SVM-RFE & Logistic asthma gene panel, and the optimal classification threshold for this model is about 0.69.

In any of the above embodiments, the classification analysis can alternatively comprise the LR-RFE & AdaBoost model as described in more detail below, the gene expression profiles analyzed by this model being the expression profiles of the genes in the LR-RFE & AdaBoost asthma gene panel, and the optimal classification threshold for this model is about 0.49.

In any of the above embodiments, the classification analysis can alternatively comprise the LR-RFE & RandomForest model as described in more detail below, the gene expression profiles analyzed by this model being the expression profiles of the genes in the LR-RFE & RandomForest asthma gene panel, and the optimal classification threshold for this model is about 0.60.

In any of the above embodiments, the classification analysis can alternatively comprise the SVM-RFE & RandomForest model as described in more detail below, the gene expression profiles analyzed by this model being the expression profiles of the genes in the SVM-RFE & RandomForest asthma gene panel, and the optimal classification threshold for this model is about 0.50.

In any of the above embodiments, the classification analysis can alternatively comprise the SVM-RFE & AdaBoost model as described in more detail below, the gene expression profiles analyzed by this model being the expression profiles of the genes in the SVM-RFE & AdaBoost asthma gene panel, and the optimal classification threshold for this model is about 0.55.

In any of the above embodiments, the patient is a mammal. In any of the above embodiments, the patient is a human.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 depicts the study flow for the identification of a nasal biomarker of asthma by machine learning analysis of next-generation transcriptomic data. Subjects with mild/moderate asthma and nonasthmatic controls were recruited for phenotyping, nasal brushing, and RNA sequencing of nasal epithelium. The RNAseq data generated were then a priori split into a development and test set. The development set was used for differential expression analysis and machine learning (involving feature selection, classification, and statistical analyses of classification performance) to identify an asthma gene panel that can accurately classify asthma from no asthma. Several classification models, including LR-RFE & Logistic, LR-RFE & SVM-Linear, SVM-RFE & Logistic, SVM-RFE & SVM-Linear, LR-RFE & AdaBoost, LR-RFE & RandomForest, SVM-RFE & RandomForest, and SVM-RFE & AdaBoost, were used to identify member genes of the asthma gene panel. The asthma gene panel identified was then tested on eight validation test sets, including (1) the RNAseq test set of subjects with and without asthma, (2) two test sets of subjects with and without asthma with nasal gene expression profiled by microarray, and (3) five test sets of subjects with non-asthma respiratory conditions (allergic rhinitis, upper respiratory infection, cystic fibrosis, and smoking) and nasal gene expression profiled by microarray. The strong precision and recall of the asthma gene panel across all test sets, reflected in the combined strong F-measure values, support its high potential to translate into a nasal brush-based biomarker for asthma diagnosis.

FIG. 2 shows the receiver operating characteristic (ROC) curve of the predictions generated by applying the asthma gene panel to the samples in the RNAseq test set of independent subjects (n=40). The ROC curve for a random model is shown for reference. The curve and its corresponding AUC score show that the panel performs well for both asthma and no asthma (control) samples in this test set.

FIG. 3 shows the validation of the asthma gene panel on test sets of independent subjects with asthma. Performance of the asthma panel in classifying asthma and no asthma in terms of Fmeasure, a conservative mean of precision and sensitivity²⁸. F-measure ranges from 0 to 1, with higher values indicating superior classification performance. The panel was applied to an RNAseq test set of independent subjects with and without asthma, and two external microarray data sets from subjects with and without asthma (Asthma1 and Asthma2).

FIG. 4 shows the comparative performance in the RNAseq test set of the LR-RFE & Logistic asthma gene panel and other classification models processed through the inventors' machine learning pipeline. Performances of the LR-RFE & Logistic asthma gene panel and other classification models in classifying asthma (left panel) and no asthma (right panel) are shown in terms of F-measure, with individual measures shown in the bars. The number of genes in each model is shown in parentheses within the bars. The LR-RFE & Logistic classification model is listed first, followed by the other classification models. These other classification models were combinations of two feature selection algorithms (LR-RFE and SVM-RFE) and four global classification algorithms (Logistic Regression, SVM-Linear, AdaBoost and Random Forest). For context, alternative classification models are also shown and include: (1) a model derived from an alternative, single-step classification approach (sparse classification model learned using the L1-Logistic regression algorithm), and (2) models substituting feature selection with each of the following preselected gene sets—all genes, all differentially expressed genes, and known asthma genes²⁹—with their respective best performing global classification algorithms. These results show the performance of the LR-RFE & Logistic asthma gene panel compared to all other models, in terms of classification performance and/or model parsimony (number of genes included). LR=Logistic Regression. SVM=Support Vector Machine. RFE=Recursive Feature Elimination. RF=Random Forest.

FIG. 5 shows the validation of the LR-RFE & Logistic asthma gene panel on test sets of independent subjects with non-asthma respiratory conditions. Performance statistics of the panel when applied to external microarray-generated data sets of nasal gene expression derived from case/control cohorts with non-asthma respiratory conditions. The LR-RFE & Logistic panel had a low to zero rate of misclassifying other respiratory conditions as asthma, supporting that the LR-RFE & Logistic panel is specific to asthma and would not misclassify other respiratory conditions as asthma.

FIG. 6 shows a heatmap showing expression profiles of the 90 gene members of the LR-RFE & Logistic asthma gene panel. Columns shaded dark grey (right-hand side) at the top denote asthma samples, while samples from subjects without asthma are denoted by columns shaded light grey (left-hand side). 22 and 24 of these genes were over- and under-expressed in asthma samples (DESeq2 FDR≤0.05), denoted by medium grey (uppermost group) and dark grey (middle group) groups of rows, respectively. The four genes in this set that have been previously associated with asthma²⁹ are C3, DEFB1, CYFIP2, and GSTT1. The LR-RFE & Logistic panel's inclusion of genes not previously known to be associated with asthma as well as genes not differentially expressed in asthma (light grey lowermost group of rows) demonstrates the ability of the inventors' machine learning methodology to move beyond traditional analyses of differential expression and current domain knowledge.

FIG. 7 shows variancePartition analysis of the RNAseq development set. Gene expression variation across RNA samples due to age, race, and sex was assessed by variancePartition and found to be minimal.

FIG. 8 shows a visual description of the machine learning pipeline used to select predictive features (genes) and develop classification models based on them from the RNAseq development set. By considering 100 splits of the development set into training and holdout sets (dotted box), many such models were evaluated for classification performance and then compared statistically using Friedman and Nemenyi tests. From this comparison, a highly precise combination of predictive genes and outer classification algorithms with good recall was determined, namely the LR-RFE & Logistic (Regression) model. This combination was in turn executed on the development set to train the LR-RFE & Logistic asthma gene panel. This LR-RFE & Logistic model was applied to several independent RNAseq and external microarray-derived cohorts with asthma and other respiratory conditions for final evaluation.

FIG. 9 shows a visual description of the feature (gene) selection component of the invented machine learning pipeline. Given a training set, this component used a 5×5 nested (outer and inner) cross-validation (CV) setup to select sets of predictive features (genes). The inner CV round was used to determine the optimal number of features to be selected, and the outer one was used to select the set of predictive genes based on this number, thus reducing the cumulative effect of these potential sources of overfitting. The selection of features itself was performed using the Recursive Feature Elimination (RFE) algorithm in combination with wrapper Logistic Regression and SVM with Linear kernel classification algorithms.

FIG. 10A-10B shows Critical Difference plots demonstrating the statistical comparison of the performance of 100 asthma classification models obtained by various combinations of feature selection and outer classification algorithms. To emphasize the need for parsimony (small feature/gene sets) in these models, an adapted performance measure defined as the F-measure for each model divided by the number of genes in that model is used for this comparison. The Friedman followed by Nemenyi tests were used to statistically compare these adapted measures and obtain the p-values constituting the above plot. Each combination is represented individually by vertical+horizontal lines on the (10A) asthma and (10B) no asthma classes constituting the RNASeq development set. Combinations with improving performance are laid out from the left to right in terms of the average rank obtained by each of their 100 models, and the combinations connected by thick black lines perform statistically equivalently. The LR-RFE & Logistic model, which identified 90 genes (listed in Table 4 below) is a highly performing combination since, on average, it achieves good performance with the fewest selected genes. Other models that performed well, along with the identified genes, are listed in Table 4 below and discussed in more detail below. Across all eight of the models, 275 unique genes were identified as listed in Table 4.

FIG. 11 shows evaluation measures for classification models. The relationships between F-measure, sensitivity, precision, recall, positive predictive value, and negative predictive value are summarized. F-measure, which is a harmonic (conservative) mean of precision and recall that is computed separately for each class, provides a more comprehensive and reliable assessment of model performance when classes are imbalanced, as is frequently the case in biomedical scenarios.

FIG. 12 shows the performance of permutation-based random classification models in test sets of independent subjects with asthma and controls. To determine the extent to which the classification performance of the LR-RFE & Logistic asthma gene panel could have been due to chance, 100 permutation-based random models were obtained by randomly permuting the labels of the samples in the development set and executing each of the feature selection-global classification combinations on these randomized data sets in the same way as described above for the real development set. These random models were then applied to each of the asthma test sets considered in our study, and their performances were also evaluated in terms of the F-measure.

FIG. 13 shows the performance of permutation-based random classification models in test sets of independent subjects with non-asthma respiratory conditions and controls. 100 permutation-based random models were obtained by randomly permuting the labels of the samples in the development set and executing each of the feature selection-global classification combinations on these randomized data sets in the same way as described above for the real development set. These random models were then applied to these test sets, and their performances were also evaluated in terms of the F-measure.

FIG. 14 shows the distribution of DESeq2 FDR values of differentially expressed genes in the LR-RFE & Logistic asthma gene panel (dark grey bars) vs. other genes in the RNAseq development set (white bars), with overlaps between the bars shown in light grey. The Y-axis shows the probability of a gene having a −log 10(FDR) value in the corresponding bin. This plot shows that the genes in the LR-RFE & Logistic asthma panel were likely to be more differentially expressed, i.e., higher −log 10(FDR) or lower differential expression FDRs, than other genes in the development set.

### DETAILED DESCRIPTION OF THE INVENTION

As specified in the Background Section, there is a great need in the art to identify technologies for reliable, consistent, simple and non-invasive diagnosis of asthma, including but not limited to mild to moderate asthma and use this understanding to develop novel diagnostic methods. The present invention satisfies this and other needs. Embodiments of the present invention relate generally to methods for diagnosis, classification and monitoring of asthma, including but not limited to mild to moderate asthma, and its differentiation from other respiratory disorders by determining the expression profiles of asthma-specific genes in nasal swab/scraping/brushing samples.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

As used herein, the term “subject” or “patient” refers to mammals and includes, without limitation, human and veterinary animals. In a preferred embodiment, the subject is human.

In the context of the present invention insofar as it relates to asthma, the terms “treat”, “treatment”, and the like mean to relieve or alleviate at least one symptom associated with such condition, or to slow or reverse the progression of such condition. Within the meaning of the present invention, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms.

The term “a control level” as used herein encompasses predetermined standards (e.g., a published value in a reference) as well as levels determined experimentally in similarly processed samples from control subjects (e.g., BMI-, age-, and gender-matched subjects without asthma as determined by standard examination and diagnostic methods). The control level is included in the classification analyses as described herein.

RNA can be extracted from the collected tissue and/or cells (e.g., from nasal epithelial cells obtained from a nasal brushing, scraping, wash, sponge or swab) by any known method. For example, RNA may be purified from cells using a variety of standard procedures as described, for example, in RNA Methodologies, A Laboratory Guide for Isolation and Characterization, 2nd edition, 1998, Robert E. Farrell, Jr., Ed., Academic Press. In addition, various commercial products are available for RNA isolation. As would be understood by those skilled in the art, total RNA or polyA+RNA may be used for preparing gene expression profiles.

The expression levels (or expression profile) can be then determined using any of various techniques known in the art and described in detail elsewhere. Such methods generally include, for example and not limitation, polymerase-based assays such as RT-PCR (e.g., TAQMAN), hybridization-based assays such as DNA microarray analysis, flap-endonuclease-based assays (e.g., INVADER), direct mRNA capture (QUANTIGENE or HYBRID CAPTURE (Digene)), RNA sequencing (e.g., Illumina RNA sequencing platforms), and by the nanoString platform. See, for example, US 2010/0190173 for descriptions of representative methods that can be used to determine expression levels.

As used herein, the term “gene” refers to a DNA sequence expressed in a sample as an RNA transcript.

As used herein, “differentially expressed” or “differential expression” means that the level or abundance of an RNA transcripts (or abundance of an RNA population sharing a common target sequence (e.g., splice variant RNAs)) is higher or lower by at least a certain value in a test sample as compared to a control level.

As used herein, the term “asthma gene panel” refers to the unique set of 275 genes identified by all of the models and listed in Table 4 as the unique set of genes. Preferred subsets of the asthma gene panel that may be analyzed by different classifiers are also described in Table 4. Specifically, as used herein, the term “LR-RFE & Logistic asthma gene panel” refers to those 90 genes identified by the LR-RFE & Logistic models. The term “LR-RFE & SVM-Linear asthma gene panel” refers to those 90 genes identified by the LR-RFE & SVM-Linear models. The term “SVM-RFE & SVM-Linear asthma gene panel” refers to those 119 genes identified by the SVM-RFE & SVM-Linear models. The term “SVM-RFE & Logistic asthma gene panel” refers to those 119 genes identified by the SVM-RFE & Logistic models. The term “LR-RFE & AdaBoost asthma gene panel” refers to those 90 genes identified by the LR-RFE & AdaBoost models. The term “LR-RFE & RandomForest asthma gene panel” refers to those 90 genes identified by the LR-RFE & RandomForest models. The term “SVM-RFE & RandomForest asthma gene panel” refers to those 123 genes identified by the SVM-RFE & RandomForest models. The term “SVM-RFE & AdaBoost asthma gene panel” refers to those 212 genes identified by the SVM-RFE & AdaBoost models.

In various embodiments disclosed herein, the expression levels of different combinations of genes can be used to glean different information. For example, increased expression levels of certain genes such as C3 in an individual as compared to a control are associated with a diagnosis of mild/moderate asthma. Decreased expression levels of other genes such as DEFB1 in an individual as compared to a control are associated with a diagnosis of mild/moderate asthma. Expression of ORMDL3 in an individual as compared to a control is associated with a differential diagnosis of mild/moderate asthma relative to other respiratory disorders such as, for example and not limitation, rhinitis, respiratory infection, and cystic fibrosis.

In various embodiments, RNA expression profiling systems are utilized to quantify the gene expression profiles from the patient's nasal brushing/swab/scraping/washing/sponge, such as for example and not limitation, the nanoString profiling system. The output from such systems will provide a count of genes in the asthma gene panel, and such output is analyzed in an automated manner, such as by a computer, via the classifier and classification threshold as described herein. The results obtained from the classifier enable a clinician to diagnose the patient as having asthma or not.

After determining and analyzing the expression levels of the appropriate combination of genes in a patient's nasal brushing/swab/scraping/washing/sponge, the patient can be classified as having asthma or not having asthma. The classification may be determined computationally based upon known methods as described herein. Particularly preferred computational methods include the classifiers and optimal classification thresholds as described herein. The result of the computation may be displayed on a computer screen or presented in a tangible form, for example, as a probability (e.g., from 0 to 100%) of the patient having asthma and/or a certain severity of asthma. The report will aid a physician in diagnosis or treatment of the patient. For example, in certain embodiments, the patient's expression levels will be diagnostic of asthma or enable a differential diagnosis of asthma from other respiratory disorders such as rhinitis, irritation resulting from smoking, respiratory infection and cystic fibrosis, and the patient will subsequently be treated as appropriate. In other embodiments, the patient's expression levels of the appropriate combination of genes will not support a diagnosis of asthma, thereby allowing the physician to exclude asthma and/or mild to moderate asthma as a diagnosis. In some embodiments, the patient may be selected to participate in clinical trials involving treatment of asthma and/or related conditions based on the patient's gene expression profile.

In some embodiments, the classifier used is the LR-RFE & Logistic model, the gene expression profiles analyzed are the expression profiles of the genes in the LR-RFE & Logistic asthma gene panel, and the optimal classification threshold for this model is about 0.76.

In other embodiments, the classifier used is the LR-RFE & SVM-Linear model, the gene expression profiles analyzed are the expression profiles of the genes in the LR-RFE & SVM-Linear asthma gene panel, and the optimal classification threshold for this model is about 0.52.

In other embodiments, the classifier used is the SVM-RFE & SVM-Linear model, the gene expression profiles analyzed are the expression profiles of the genes in the SVM-RFE & SVM-Linear asthma gene panel, and the optimal classification threshold for this model is about 0.64.

In other embodiments, the classifier used is the SVM-RFE & Logistic model, the gene expression profiles analyzed are the expression profiles of the genes in the SVM-RFE & Logistic asthma gene panel, and the optimal classification threshold for this model is about 0.69.

In other embodiments, the classifier used is the LR-RFE & AdaBoost model, the gene expression profiles analyzed are the expression profiles of the genes in the LR-RFE & AdaBoost asthma gene panel, and the optimal classification threshold for this model is about 0.49.

In other embodiments, the classifier used is the LR-RFE & RandomForest model, the gene expression profiles analyzed are the expression profiles of the genes in the LR-RFE & RandomForest asthma gene panel, and the optimal classification threshold for this model is about 0.60.

In other embodiments, the classifier used is the SVM-RFE & RandomForest model, the gene expression profiles analyzed are the expression profiles of the genes in the SVM-RFE & RandomForest asthma gene panel, and the optimal classification threshold for this model is about 0.50.

In other embodiments, the classifier used is the SVM-RFE & AdaBoost model, the gene expression profiles analyzed are the expression profiles of the genes in the SVM-RFE & AdaBoost asthma gene panel, and the optimal classification threshold for this model is about 0.55.

In some embodiments, RNAs are purified prior to gene expression profile analysis. RNAs can be isolated and purified from nasal brushing/swab/scraping/wash/sponge by various methods, including the use of commercial kits (e.g., Qiagen RNeasy Mini Kit as described in Example 1 below). In some embodiments, RNA degradation in brushing/swab/scraping/wash/sponge samples and/or during RNA purification is reduced or eliminated. Useful methods for storing nasal brushing/swab/scraping/wash/sponge samples include, without limitation, use of RNALater as described herein. Useful methods for reducing or eliminating RNA degradation include, without limitation, adding RNase inhibitors (e.g., RNasin Plus [Promega], SUPERase-In [ABI], etc.), use of guanidine chloride, guanidine isothiocyanate, N-lauroylsarcosine, sodium dodecylsulphate (SDS), or a combination thereof. Reducing RNA degradation in nasal brushing/swab/scraping/wash/sponge samples is particularly important when sample storage and transportation is required prior to RNA purification.

In other embodiments, RNA is not purified prior to gene expression profile analysis. In such embodiments, RNA expression profiling platforms that can directly assay tissue and cells without a separate RNA isolation step are utilized (for example and not limitation, the nanoString system).

Examples of useful methods for measuring RNA level in nasal epithelial cells contained in nasal brushing/swab/scraping/wash/sponge include hybridization with selective probes (e.g., using Northern blotting, bead-based flow-cytometry, oligonucleotide microchip [microarray], or solution hybridization assays), polymerase chain reaction (PCR)-based detection (e.g., stem-loop reverse transcription-polymerase chain reaction [RT-PCR], quantitative RT-PCR based array method [qPCR-array]), direct sequencing, such as for example and not limitation, by RNA sequencing technologies (e.g., Illumina HiSeq 2500 platform, Helicos small RNA sequencing, miRNA BeadArray (Illumina), Roche 454 (FLX-Titanium), and ABI SOLiD), and the nanoString system. For review of additional applicable techniques see, e.g., Chen et al., BMC Genomics, 2009, 10:407; Kong et al., J Cell Physiol. 2009; 218:22-25.

In conjunction with the above diagnostic and screening methods, the present invention provides various kits comprising one or more primer and/or probe sets specific for the detection of target RNA. Such kits can further include primer and/or probe sets specific for the detection of other RNA that can aid in diagnosing, differentiating, and/or classifying asthma. In some embodiments, such kits can contain nucleic acid oligonucleotides for determining the level of expression of a particular combination of genes in a patient's nasal brushing/swab/scraping/wash/sponge sample. The kit may include one or more oligonucleotides that are complementary to one or more transcripts identified herein as being associated with asthma, and also may include oligonucleotides related to necessary or meaningful assay controls. A kit for evaluating an individual for asthma may include pairs of oligonucleotides (e.g., 4, 6, 8, 10, 12, 14 or more oligonucleotides). The oligonucleotides may be designed to detect expression levels in accordance with any assay format, including but not limited to those described herein. The kit may further optionally include control primer and/or probe sets for detection of control RNA in order to provide a control level as described herein.

A kit of the invention can also provide reagents for primer extension and amplification reactions. For example, in some embodiments, the kit may further include one or more of the following components: a reverse transcriptase enzyme, a DNA polymerase enzyme (such as, e.g., a thermostable DNA polymerase), a polymerase chain reaction buffer, a reverse transcription buffer, and deoxynucleoside triphosphates (dNTPs). Alternatively (or in addition), a kit can include reagents for performing a hybridization assay. The detecting agents can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. In addition, the kit may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. A kit can further include RNA isolation or purification means as well as positive and negative controls. A kit can also include a notice associated therewith in a form prescribed by a governmental agency regulating the manufacture, use or sale of diagnostic kits. Detailed instructions for use, storage and trouble-shooting may also be provided with the kit. A kit can also be optionally provided in a suitable housing that is preferably useful for robotic handling in a high throughput setting.

The components of the kit may be provided as dried powder(s). When reagents and/or components are provided as a dry powder, the powder can be reconstituted by the addition of a suitable solvent. It is envisioned that the solvent may also be provided in another container. The container will generally include at least one vial, test tube, flask, bottle, syringe, and/or other container means, into which the solvent is placed, optionally aliquoted. The kits may also comprise a second container means for containing a sterile, pharmaceutically acceptable buffer and/or other solvent.

Where there is more than one component in the kit, the kit also will generally contain a second, third, or other additional container into which the additional components may be separately placed. However, various combinations of components may be comprised in a container.

Such kits may also include components that preserve or maintain DNA or RNA, such as reagents that protect against nucleic acid degradation. Such components may be nuclease or RNase-free or protect against RNases, for example. Any of the compositions or reagents described herein may be components in a kit.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning. A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Development of the Nasal Biomarker Panel

### Materials and Methods

Experimental Design and Subjects

Subjects with mild/moderate asthma were a subset of participants of the Childhood Asthma Management Program (CAMP), a multicenter North American clinical trial of 1041 subjects that took place between 1991 and 2012^{21,22}. Findings from the CAMP cohort have defined current practice and guidelines for asthma care and research²². Participating subjects had asthma defined by symptoms greater than or equal to 2 times per week, use of an inhaled bronchodilator at least twice weekly or use of daily medication for asthma, and increased airway responsiveness to methacholine (PC₂₀≤12.5 mg/ml). The subset of subjects included in this study were CAMP participants who presented for a visit between July 2011 and June 2012 at Brigham and Women's Hospital, one of eight study centers for this multicenter study.

Subjects without asthma or “no asthma” were recruited during the same time period (2011-2012) by advertisement at Brigham & Women's Hospital. Selection criteria were no personal history of asthma, no family history of asthma in first degree relatives, and self-described non-Hispanic white ethnicity. The rationale for limiting participation to non-Hispanic white individuals was to allow for optimal comparison to 968 CAMP subjects of Caucasian background who participated in the CAMP Genetics Ancillary study, which was focused on this population.⁵⁵ Subjects underwent pre and post-bronchodilator spirometry according to ATS guidelines, and only those meeting selection criteria and without lung function abnormality or bronchodilator response were considered nonasthmatic or “no asthma”.

The institutional review boards of Brigham & Women's Hospital and the Icahn School of Medicine at Mount Sinai approved the study protocols.

Nasal Sample Collection and RNA Sequencing

A standard cytology brush was applied to the right nare of each subject and rotated three times with circumferential pressure for nasal epithelial cell collection. The brush was immediately placed in RNALater and then stored at 4° C. until RNA extraction. RNA extraction was performed with Qiagen RNeasy Mini Kit (Valencia, Calif.). Samples were assessed for yield and quality using the 2100 Bioanalyzer (Agilent Technologies, Santa Clara, Calif.) and Qubit (Thermo Fisher Scientific, Grand Island, N.Y.).

Of the 190 subjects who underwent nasal brushing (66 with mild/moderate asthma, 124 with no asthma), a random selection of 150 nasal brushes from subjects with asthma and nonasthmatic controls were a priori assigned as the development set, and the remaining 40 subjects were a priori assigned as the test set of independent subjects (for testing the classification model). To minimize potential bias due to batch effects, the inventors submitted all samples (training and test set samples) to the Mount Sinai Genomics Core for library preparation and RNA sequencing at the same time to allow for sequencing of all samples in a single run. Staff at the Mount Sinai Genomics Core were blinded to the assignment of samples as development or test set.

The sequencing library was prepared with the standard TruSeq RNA Sample Prep Kit v2 protocol (Illumina). The mRNA sequencing was performed on the Illumina HiSeq 2500 platform using 40-50 million 100 bp paired-end reads. The data were put through the inventors' standard mapping pipeline⁵⁶ (using Bowtie⁵⁷ and TopHat⁵⁸, and assembled into gene- and transcription-level summaries using Cufflinks⁵⁹). Mapped data were subjected to quality control with FastQC and RNA-SeQC.⁶⁰ Data were normalized separately for the development and test sets. Genes with fewer than 100 counts in at least half the samples were dropped to reduce the potentially adverse effects of noise. DESeq2²⁵ was used to normalize the data sets using its variance stabilizing transformation method.

VariancePartition Analysis of Potential Confounders

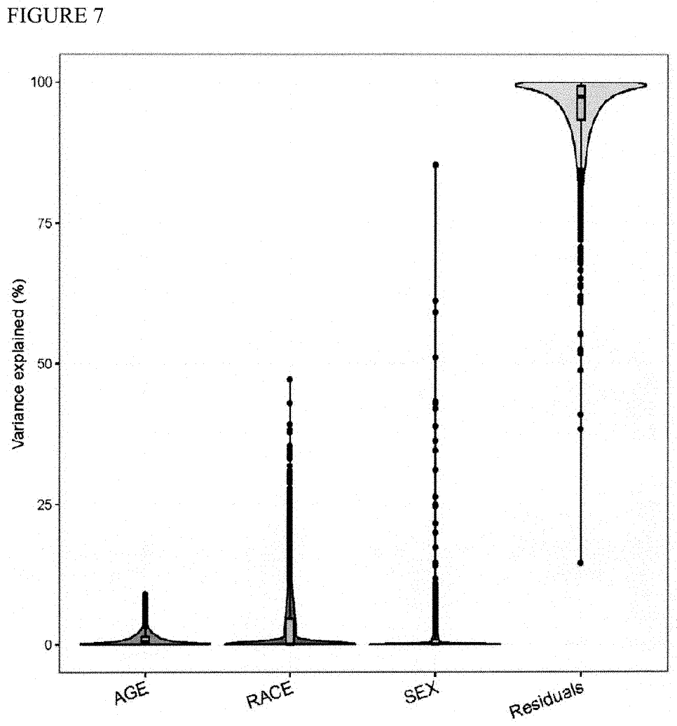

Given differences in age, race, and sex distributions between the asthma and “no asthma” classes, the inventors used variancePartition²⁴ to assess the degree to which these variables influenced gene expression. The total variance in gene expression was partitioned into the variance attributable to age, race, and sex using a linear mixed model implemented in variancePartition v1.0.0²⁴. Age (continuous variable) was modeled as a fixed effect while race and sex (categorical variables) were modeled as random effects. The results showed that age, race, and sex accounted for minimal contributions to total gene expression variance (FIG. 7).

Downstream Analyses were Therefore Performed with Unadjusted Gene Expression Data.

Differential gene expression and pathway enrichment analysis DESeq2²⁵ was used to identify differentially expressed genes in the development set. Genes with FDR≤0.05 were deemed differentially expressed, with fold change <1 implying under-expression and vice versa. Pathway enrichment analysis was performed using Gene SetEnrichment Analysis²⁶.

Statistical and Machine Learning Analyses of RNAseq Data Sets

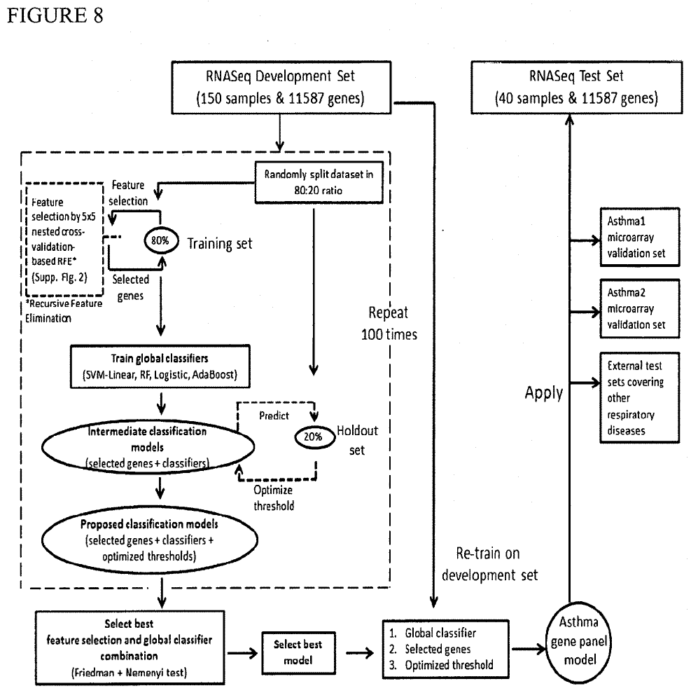

To discover gene expression biomarkers that are capable of predicting the asthma status of a patient, the inventors used a rigorous machine learning pipeline in Python using the scikit-learn package⁶¹. This pipeline combined feature (gene) selection¹⁸, (outer) classification¹⁹ and statistical analyses of classification performance²⁰ to the development set (FIG. 8). The first two components, feature selection and classification, were applied to a training set constituted of 120 randomly selected samples from the development set (n=150) to learn classification models. These models were evaluated on the corresponding remaining 30 samples (holdout set). This process (feature selection and classification) was repeated 100 times on 100 random splits of the development set into training and holdout sets.

Feature (Gene) Selection:

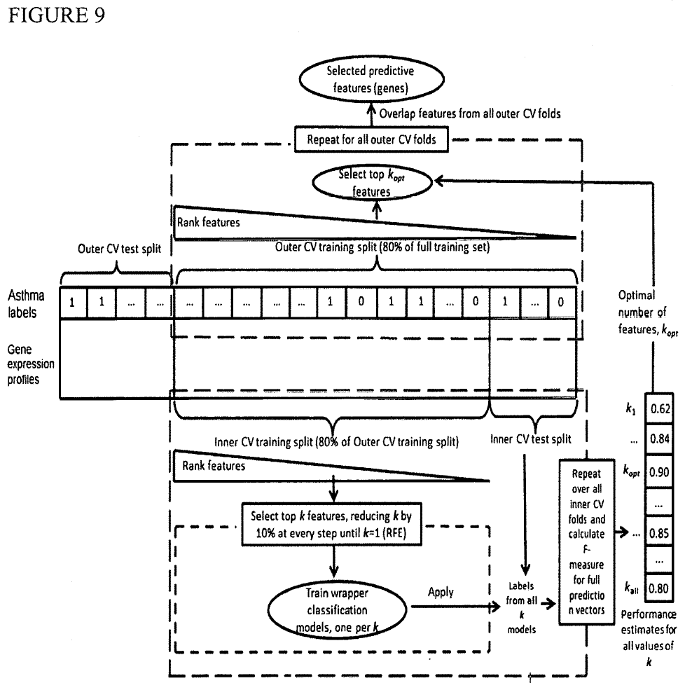

Given a training set, a 5×5 nested (outer and inner) cross-validation (CV) setup²⁷ was used to select sets of predictive genes (FIG. 9). The inner CV round was used to determine the optimal number of genes to be selected, and the outer CV round was used to select the set of predictive genes based on this number, thus reducing the cumulative effect of these potential sources of overfitting.

The Recursive Feature Elimination (RFE) algorithm⁶² was executed on the inner CV training split to determine the optimal number of features. The use of RFE within this setting enabled the inventors to identify groups of features that are collectively, but not necessarily individually, predictive. This reflects the systems biology-based expectation that many genes, even ones with marginal effects, can play a role in classifying diseases/phenotypes (here asthma) in combination with other more strongly predictive genes⁶³. Specifically, the inventors used the L2-regularized Logistic Regression (LR or Logistic)⁶⁴ and SVM-Linear(kernel)⁶⁵ classification algorithms in conjunction with RFE (conjunctions henceforth referred to as LR-RFE and SVM-RFE respectively). For this, for a given inner CV training split, all the features (genes) were ranked using the absolute values of the weights assigned to them by an inner classification model, trained using the LR or SVM algorithm, over this split. Next, for each of the conjunctions, the set of top-k ranked features, with k starting with 11587 (all filtered genes) and being reduced by 10% in each iteration until k=1, was considered. The discriminative strength of feature sets consisting of the top k features as per this ranking was assessed by evaluating the performance of the LR or SVM classifier based on them over all the inner CV training-test splits. The optimal number of features to be selected was determined as the value of k that produces the best performance. Next, a ranking of features was derived from the outer CV training split using exactly the same procedure as applied to the inner CV training split. The optimal number of features determined above was selected from the top of this ranking to determine the optimal set of predictive features for this outer CV training split. Executing this process over all the five outer CV training splits created from the development set identified five such sets. Finally, the set of features (genes) that was common to all these sets (i.e., in their intersection/overlap) was selected as the predictive gene set for this training set. One such set was identified for LR-RFE and SVM-RFE respectively.

(Outer) Classification:

Once respective predictive gene sets had been selected using LR-RFE and SVM-RFE, four outer classification algorithms, namely L2-regularized Logistic Regression (LR or Logistic)⁶⁴, SVM-Linear⁶⁶, AdaBoost⁶⁶ and Random Forest (RF)⁶⁷, were used to learn intermediate classification models over the training set. These intermediate models were applied to the corresponding holdout set to generate probabilistic asthma predictions for the constituent samples. An optimal threshold for converting these probabilistic predictions into binary ones was then computed from the holdout set. This optimization resulted in the proposed classification models. This optimization resulted in proposed classification models.

To obtain a comprehensive view of the performance of these proposed models, the above two components were executed on 100 random training-holdout splits of the development set. To determine the best performing combination of feature selection and outer classification algorithms, a statistical analysis of the classification performance of all the models resulting from all the considered combinations was conducted using the Friedman followed by the Nemenyi test^{20,68} These tests, which account for multiple hypothesis testing, assessed the statistical significance of the relative difference of performance of the combinations in terms of their relative ranks across the 100 splits, and allow the ordering of the overall performance of each combination in terms of the significance of their pairwise comparison. This statistical comparison was a novel aspect of the present pipeline, as this task, generally referred to as “model selection,” is typically based on a single training-holdout split. Even if multiple such splits are employed, models are generally selected based on absolute performance scores, and not based on the statistical significance of performance comparisons, as was done in the present Examples.

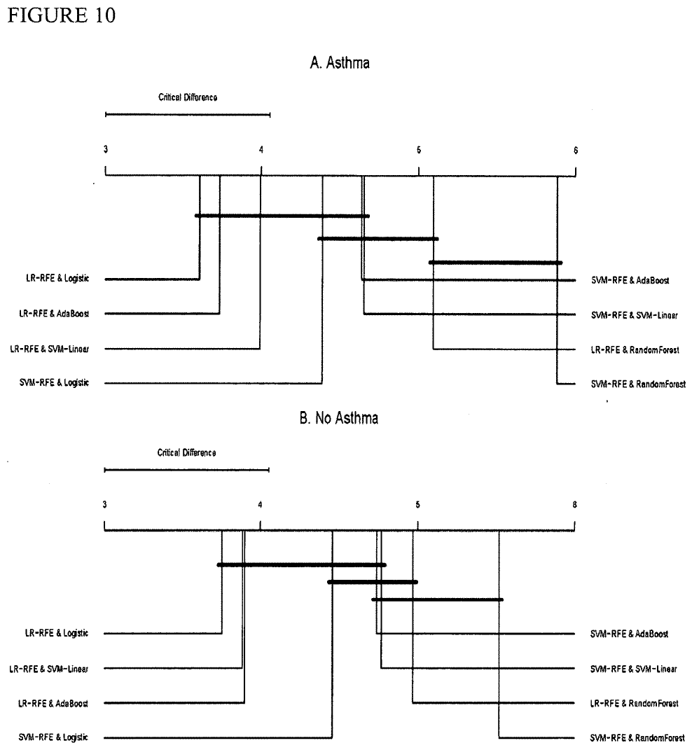

Optimization for parsimony: For biomarker optimization, it is essential to consider parsimony (i.e., minimize number of features or genes for accurate classification) In these models, an adapted performance measure, defined as the absolute performance measure for each model divided by the number of genes in that model, was used for this statistical comparison. In terms of this measure, a model that does not obtain the best absolute performance measure among all models, but uses much fewer genes than the other, may be judged to be the best model. The result of this statistical analysis, visualized as a Critical Difference plot²⁸ (FIG. 10A-10B), enabled identification of the good-performing combination of feature selection and outer classification methods in terms of both performance and parsimony.

Final Model Development and Evaluation:

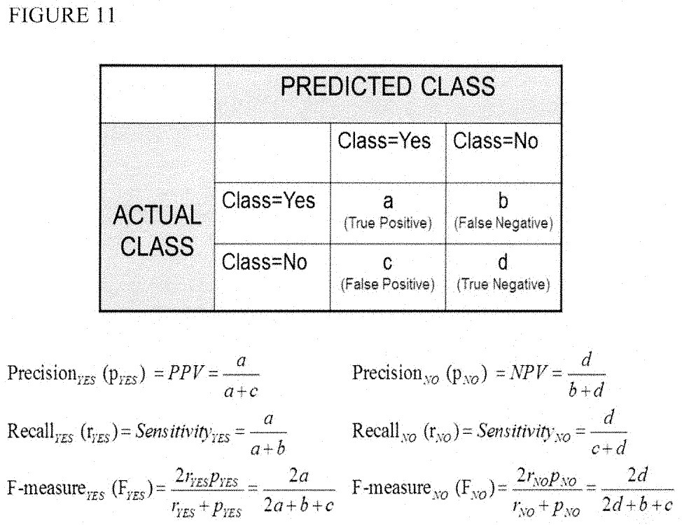

The final step in the pipeline was to determine the representative model from the 100 iterations of the most statistically superior combination of feature selection and classification method identified from the above steps. In case of ties among the models of the best performing combination, the gene set that produced the best asthma classification F-measure (FIG. 11) across all four global classification algorithms was chosen as the gene set constituting the representative model for that combination. The result of this process was the asthma gene panel-based model that consisted of this representative gene set for each of eight models, a global classification algorithm and each model's optimized threshold for classifying samples with and without asthma. This optimized threshold was determined for this model as the one that produced the highest F-measure for the asthma class on the holdout set from which it was identified. The gene sets for each of the eight models are shown in Table 4 below, as well as the 275 unique genes in the asthma gene panel are also shown.

Validation of the LR-RFE & Logistic Asthma Gene Panel in an RNAseq Test Set of Independent Subjects

The LR-RFE & Logistic asthma gene panel identified by the machine learning pipeline was then tested on the RNAseq test set (n=40) to assess its performance in independent subjects. F-measure was used to measure performance. For comparison, the same machine learning methodology was used to train and evaluate models from all combinations of feature selection and classification methods considered in the pipeline.

LR-RFE & Logistic Performance Comparison to Alternative Classification Models

To evaluate the relative performance of the LR-RFE & Logistic asthma gene panel, the inventors also applied the machine learning pipeline with replacement of the feature (gene) selection step with these pre-determined gene sets: (1) all filtered RNAseq genes, (2) all differentially expressed genes, and (3) known asthma genes from a recent review of asthma genetics²⁹. These were each used as a predetermined gene set that was run through our machine learning pipeline (FIG. 8 with the feature selection component turned off) to identify the best performing global classification algorithm and the optimal asthma classification threshold for this predetermined set of features. The algorithm and threshold were used to train this gene set's representative classification model over the entire development set, and the optimal model for each of these gene sets was then evaluated on the RNAseq test set in terms of the F-measures for the asthma and no asthma classes. Finally, as a baseline representative of sparse classification algorithms, which represent a one-step option for doing feature selection and classification simultaneously, the inventors also trained an L1-regularized logistic regression model (L1-Logistic)⁶⁹ on the development set and evaluated it on the RNAseq test set.

Performance Comparison to Permutation-Based Random Models

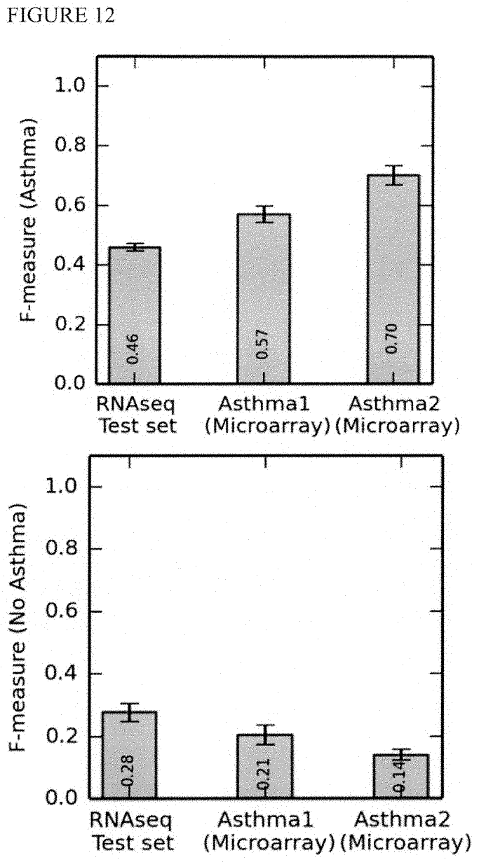

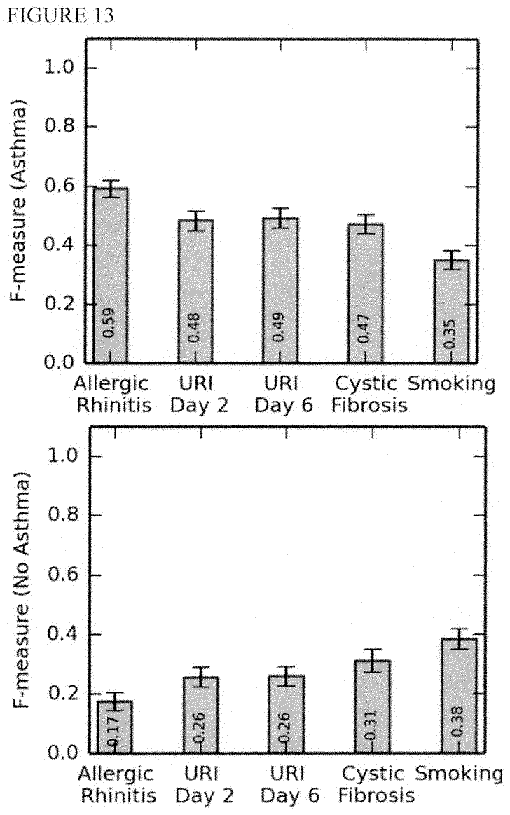

To determine the extent to which the performance of all the above classification models could have been due to chance, the inventors compared their performance with that of random counterpart models (FIGS. 12, 13). These models were obtained by randomly permuting the labels of the samples in the development set and executing each of the feature selection-global classification combinations on these randomized data sets in the same way as described above for the real development set. These random models were then applied to each of the test sets considered in our study, and their performances were also evaluated in terms of the F-measure. For each of real models trained using the combinations, 100 corresponding random models were learned and evaluated as above, and the performance of the real model was compared with the average performance of the corresponding random models.

Validation of the Asthma Gene Panel in External Asthma Cohorts

To assess the generalizability of the asthma gene panel, microarray-profiled data sets of nasal gene expression from two external asthma cohorts—Asthma1 (GSE19187)³⁰ and Asthma2 (GSE46171)³¹ (Table 5)—were obtained from NCBI Gene Expression Omnibus (GEO)⁷⁰. The asthma gene panel was evaluated on these external asthma test sets with performance measured by F-measures for the asthma and no asthma classes.

Validation of the Asthma Gene Panel in External Cohorts with Other Respiratory Conditions

To assess the panel's ability to distinguish asthma from respiratory conditions that can have overlapping symptoms with asthma, microarray-profiled data sets of nasal gene expression were also obtained for five external cohorts with allergic rhinitis (GSE43523)³⁶, upper respiratory infection (GSE46171)³¹, cystic fibrosis (GSE40445)³⁷, and smoking (GSE8987)¹² (Table 6). The asthma gene panel was evaluated on these external test sets of non-asthma respiratory conditions with performance measured by F− measures for the asthma and no asthma classes.

### Results

Study Population and Baseline Characteristics

A total of 190 subjects underwent nasal brushing for this study, including 66 subjects with well-defined mild-moderate asthma (based on symptoms, medication use, and demonstrated airway hyperresponsiveness by methacholine challenge response) and 124 subjects without asthma (based on no personal or family history of asthma, normal spirometry, and no bronchodilator response). The definitional criteria we used for mild-moderate asthma were consistent with US National Heart Lung Blood Institute guidelines for the diagnosis of asthma⁷, and are the same criteria used in the longest NIH-sponsored study of mild-moderate asthma^{21,22}.

From these 190 subjects, a random selection of 150 subjects were a priori assigned as the development set (to be used for classification model development and biomarker identification), and the remaining 40 subjects were a priori assigned as the RNAseq test set (to be used as one of 8 validation test sets for testing of the classification model and biomarker genes identified with the development set). Assignment of subjects to the development and test sets was done at this early juncture in the study to enable RNA sequencing from all subjects in a single run (to reduce potential bias from sequencing batch effects) with then immediate allocation of the sequence data to the development or test sets prior to any pre-processing and analysis. The test set was then set aside to preserve its independence.

The baseline characteristics of the subjects in the development set (n=150) are shown in the left section of Table 1. The mean age of subjects with and without asthma was comparable, with slightly more male subjects with asthma and more female subjects without asthma. Caucasians were more prevalent in subjects without asthma, which was expected based on the inclusion criteria. Consistent with the reversible airway obstruction that characterizes asthma⁴, subjects with asthma had significantly greater bronchodilator response than control subjects (P=1.4×10−5). Allergic rhinitis was more prevalent in subjects with asthma (P=0.005), consistent with known comorbidity between allergic rhinitis and asthma²³. Rates of smoking between subjects with and without asthma were not significantly different.

RNA isolated from nasal brushings from the subjects was of good quality with mean RIN 7.8 (±1.1). The median number of paired-end reads per sample from RNA sequencing was 36.3 million. Following normalization and filtering, 11,587 genes were used for analysis. VariancePartition analysis²⁴ showed that age, race, and sex minimally contributed to total gene expression variance (FIG. 7).

TABLE 1
Baseline characteristics of subjects in the RNAseq development and test sets

| | Development Set | | | | | | Test Set | | | | | | |
|---|---|---|---|---|---|---|---|---|---|---|---|---|---|
| | | | | | No | | | | | | No | | Development |
| | All | | Asthma | | Asthma | | All | | Asthma | | Asthma | | vs. test Set P |
| | (n = 150) | | (n = 53) | | (n = 97) | | (n = 40) | | (n = 13) | | (n = 27) | | value^{B} |
| Age (years) | 26.9 | (5.4) | 25.7 | (2.0) | 27.6 | (6.5) | 26.2 | (5.1) | 25.3 | (2.1) | 26.6 | (6.1) | 0.47 |
| Sex - female | 89 | (59.3%) | 24 | (45.3%) | 65 | (67.0%) | 21 | (52.5%) | 2 | (15.3%) | 19 | (70.4%) | 0.40 |
| Race | | | | | | | | | | | | | 0.60 |
| Caucasian | 116 | (77.3%) | 21 | (40.4%) | 96 | (99.0%) | 32 | (80.0%) | 5 | (38.5%) | 27 | (100.0%) | |
| African | 24 | (16.0%) | 23 | (43.4%) | 1 | (1.0%) | 32 | (80.0%) | 5 | (38.5%) | 0 | (0.0%) | |
| American | | | | | | | | | | | | | |
| Latino | 5 | (3.3%) | 5 | (9.4%) | 0 | (0.0%) | 5 | (12.5%) | 5 | (38.5%) | 0 | (0.0%) | |
| Other | 5 | (3.3%) | 4 | (7.5%) | 0 | (0.0%) | 0 | (0.0%) | 0 | (0.0%) | 0 | (0.0%) | |
| FEV1^{A} (% | 94.7% | (10.0%) | 94.6% | (10.9%) | 94.8% | (9.7%) | 94.5% | (11.4%) | 94.4% | (12.0%) | 94.6 | (11.3%) | 0.90 |
| predicted) | | | | | | | | | | | | | |
| FEV1/FVC^{A} | 82.5% | (6.4%) | 81.5% | (6.7%) | 83.1% | (6.3%) | 82.7% | (5.5%) | 84.8% | (4.4%) | 81.6% | (5.8%) | 0.91 |
| (% predicted) | | | | | | | | | | | | | |
| Bronchodilator | 5.6% | (6.0%) | 8.7% | (6.4%) | 3.9% | (5.1%) | 4.5% | (5.4%) | 7.0% | (6.1%) | 3.3% | (4.7%) | 0.29 |
| response | | | | | | | | | | | | | |
| (%) | | | | | | | | | | | | | |
| Age asthma | | | 3.2 | (2.7) | n/a | | | | 3.4 | (2.0) | | | 0.78 |
| onset: years | | | | | | | | | | | | | |
| Allergic | 60 | (40.0%) | 29 | (54.7%) | 31 | (32.0%) | 7 | (17.5%) | 7 | (53.8%) | 0 | (0%) | 0.009 |
| rhinitis | | | | | | | | | | | | | |

| Nasal | 14 | (9.3%) | 9 | (170%) | 5 | (5.2%) | 0 | | 0 | 0 | | 0.07 |
|---|---|---|---|---|---|---|---|---|---|---|---|---|
| steroids | | | | | | | | | | | | |
| Smoking | 7 | (4.7%) | 1 | (1.9%) | 6 | (6.2%) | 1 | (2.5%) | 0 | 1 | (3.7%) | 1.0 |

^{A}pre-bronchodilator measures. FEV1 = forced expiratory flow volume in 1 second, FVC = forced vital capacity. Mean (SD) or Number (%) provided.
^{B}Fisher's Exact test for categorical variables and t-test for continuous variables.

Differential gene expression analysis by DeSeq2²⁵, showed that 1613 and 1259 genes were respectively over- and under-expressed in asthma cases versus controls (false discovery rate (FDR)≤0.05) (Table 2A-2B). These genes were enriched for disease-relevant pathways²⁶ including immune system (fold change=3.6, FDR=1.07×10−22), adaptive immune system (fold change=3.91, FDR=1.46×10−15), and innate immune system (fold change=4.1, FDR=4.47×10−9) (Table 2A-2B).

Identification of the Asthma Gene Panel by Machine Learning Analyses of RNAseq Development Set

To identify gene expression biomarkers that accurately predict asthma status, the inventors developed a nested machine learning pipeline that combines feature (gene) selection¹⁸ and classification¹⁹ techniques (FIG. 8). The first component of the pipeline used a nested (inner and outer) cross-validation protocol²⁷ for selecting predictive sets of features (FIG. 8). For this, the inventors used the Recursive Feature Elimination (RFE) algorithm¹⁸ combined with L2-regularized Logistic Regression (LR or Logistic) and Support Vector Machine (SVM (with Linear kernel))¹⁹ classification algorithms (the combinations are referred to as LR-RFE and SVM-RFE respectively). Asthma classification models were then learned by applying four global classification algorithms (SVM-Linear, AdaBoost, Random Forest, and Logistic) to the expression profiles of the selected genes. This learning and evaluation process was run over 100 training-holdout splits of the development set. All resulting models were statistically compared²⁰ in terms of their performance and parsimony (i.e., number of feature/gene sets included in the model) (FIG. 10A-10B). Performance was measured in terms of F-measure²⁸, a conservative mean of precision and sensitivity. F-measure ranges from 0 to 1, with higher values indicating superior classification performance. A value of 0.5 for F-measure does not represent a random model. To estimate random performance, the inventors trained and evaluated permutation-based random models as described herein. Given the central role that F-measure plays in the interpretation of these results, a detailed explanation of F-measure and its relation to more common performance measures is provided below and in FIG. 11.

Evaluation Measures for Predictive Models

The most commonly used evaluation measures for predictive models in medicine are the positive and negative predictive values (PPV and NPV respectively). As shown in FIG. 11, PPV and NPV are equivalent to precisions²⁸ for the positive and negative classes (asthma and no asthma in our study) respectively. However, relying solely on predictive values (i.e., precisions) ignores the critical dimension of the sensitivity or recall²⁸ (also defined in FIG. 11) of the test. For instance, the test may predict perfectly for only one asthma sample in a cohort and make no predictions for all other asthma samples. This will yield a PPV of 1, but poor sensitivity/recall. Thus, for all tasks involving evaluation of asthma classification models in our study, F-measure (FIG. 11) was used as the main performance measure. This measure, which is a harmonic (conservative) mean of precision and recall that is computed separately for each class, provides a more comprehensive and reliable assessment of model performance. Furthermore, unlike area under the receiver operating characteristic (ROC) curve (AUC), F-measure is the preferred metric for classification performance when case and control groups are not balanced (i.e., 1:1)²⁸, which is frequently the case in clinical studies and medical practice. Like AUC, F-measure ranges from 0 to 1, with higher values indicating superior classification performance. However, unlike AUC, a value of 0.5 for F-measure does not represent a random model and could in some cases indicate superior performance over random. F-measures for random performance for specific datasets and models can be estimated using permutation-based random models as described herein.

A combination with good precision and recall determined from this comparison was LR-RFE & Logistic (FIG. 10A, 10B), as the models learned using this feature selection and classification model were able to obtain the best performance with the fewest number of selected genes. This combination used the logistic regression algorithm¹⁹ as both the feature selection algorithm and global classification algorithm. The model learned using this combination, built upon an optimal set of 90 predictive genes, had perfect F-measures (F=1.00) in classifying asthma and no asthma in its corresponding holdout set. This model also significantly outperformed permutation-based random models The other seven classification models listed in Table 4 also had good precision and recall with the asthma gene panel.

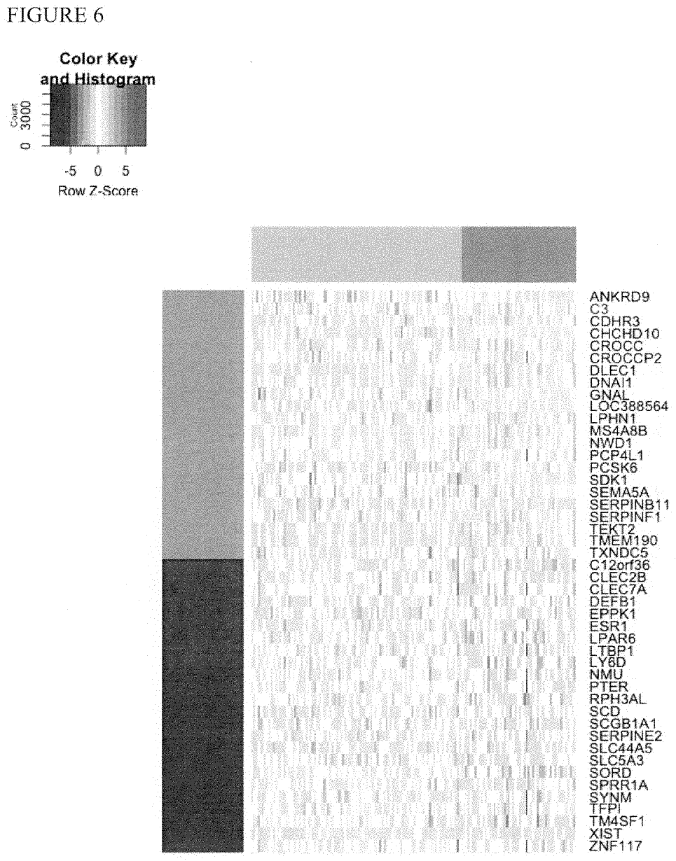

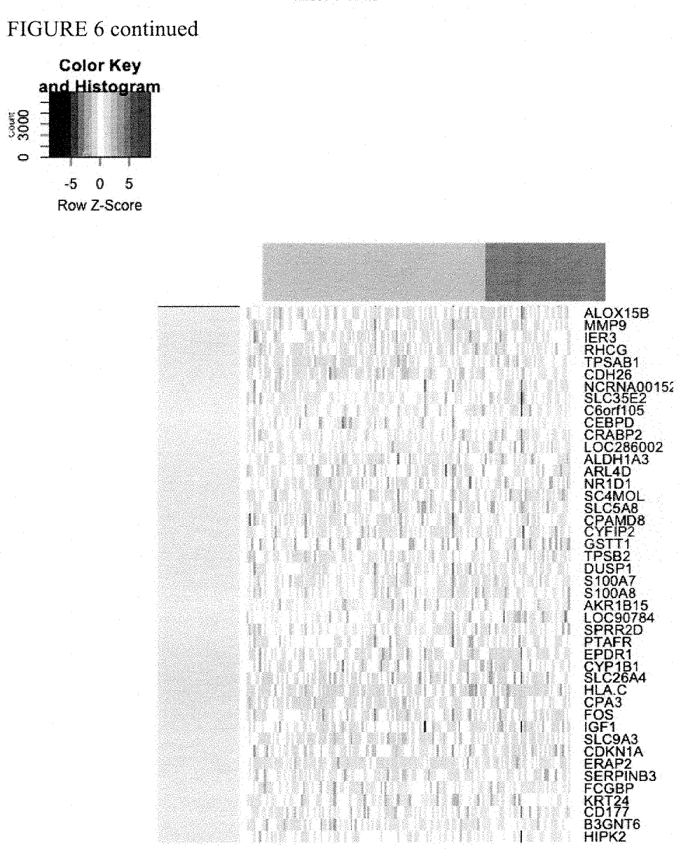

Forty six of the 90 genes included in the LR-RFE & Logistic model were differentially expressed genes, with 22 and 24 genes over- and under-expressed in asthma, respectively (FIG. 6 and Table 2A-2B). The remaining 44 genes were not differentially expressed. These results support that the machine learning pipeline was able to extract information beyond differentially expressed genes, allowing for the identification of a parsimonious panel of genes that together allowed for accurate asthma classification. Among these 90 genes, only four (C3, DEFB1, CYFIP2 and GSTT1) are known asthma genes³⁷. This demonstrates that the invented methodology effectively mines data to discover predictive genes that would not have been found by relying exclusively on current domain knowledge.

The LR-RFE & Logistic model of 90 genes is a subset of the 275 unique genes identified in all eight models, which 275 genes are defined as the “asthma gene panel”. Preferably, the 90 genes in this LR-RFE & Logistic asthma gene panel are used in combination with the LR-RFE & Logistic classifier and the model's optimal classification threshold (classify as asthma if probability output ≥about 0.76, else no asthma) to be effectively used for asthma classification, diagnosis or detection. Similarly, the genes in the model-specific asthma gene panels (Table 4) are used in combination with their model-specific classifiers and the model-specific optimal classification threshold to classify, diagnose or detect asthma effectively.

Validation of the Asthma Gene Panel in an RNAseq Test Set of Independent Subjects

The inventors tested the asthma gene panel identified from the above-described machine learning pipeline on an independent RNAseq test set. For this step, the inventors used the test set (n=40) of nasal RNAseq data from independent subjects that was set aside and remained untouched by the development set analysis. The baseline characteristics of the subjects in the test set (n=40) are shown in the right section of Table 1. The baseline characteristics were similar between the development and test sets, except for a lower prevalence of allergic rhinitis among those without asthma in the test set.

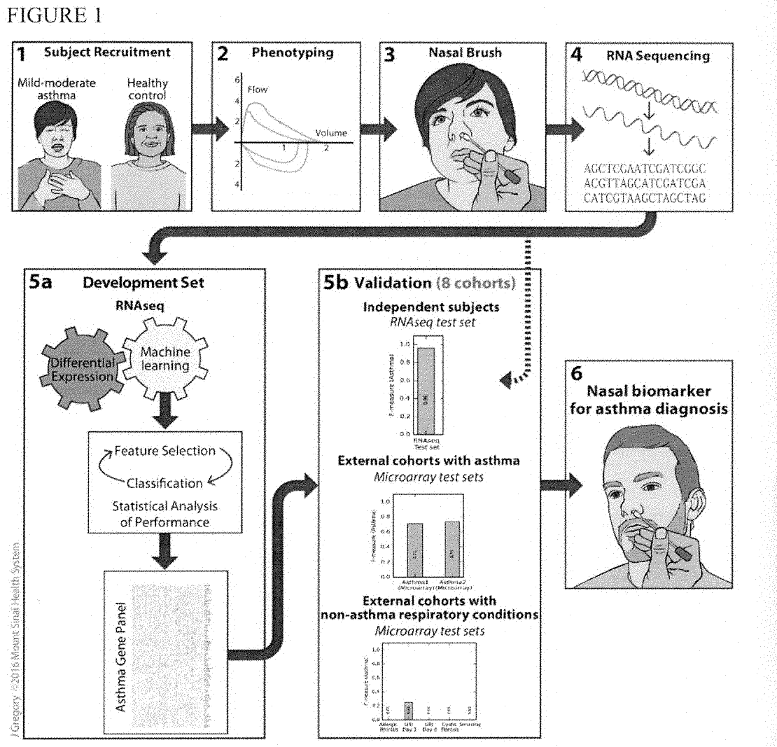

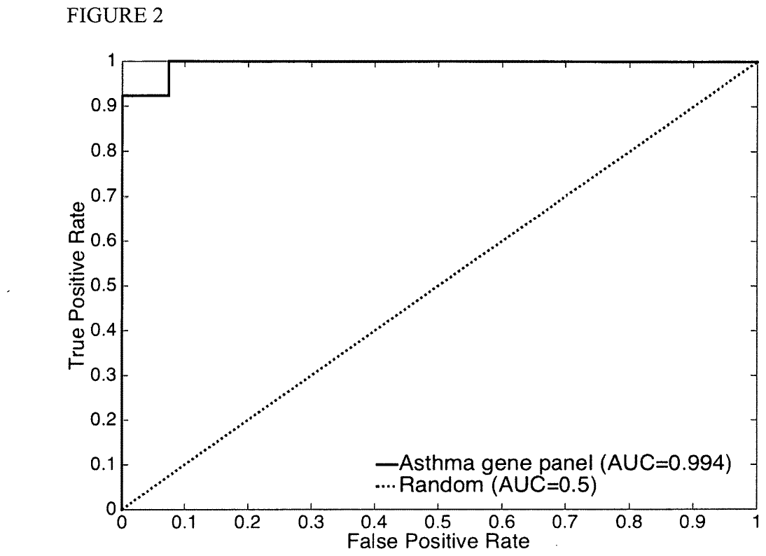

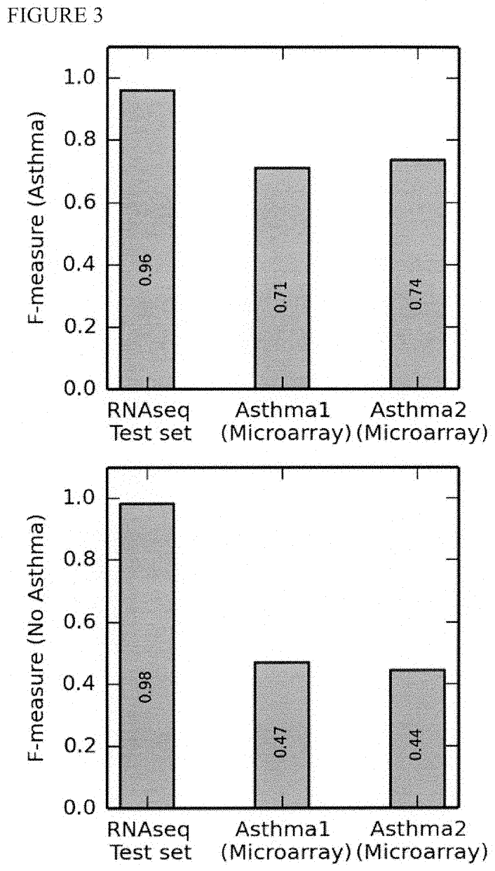

The LR-RFE & Logistic Model asthma gene panel performed with high accuracy in the RNAseq test set of independent subjects, achieving AUC=0.994 (FIG. 2). The panel achieved high positive predictive value (PPV) of 1.00 and negative predictive value (NPV) of 0.96. Given imbalances in the case and control groups, F-measure is the preferred and more conservative metric for classification performance (FIG. 1). The asthma gene panel achieved F=0.98 and 0.96 for classifying asthma and no asthma respectively (FIG. 3, left set of bars). For comparison, the much lower performance of permutation-based random models is shown in FIG. 12.

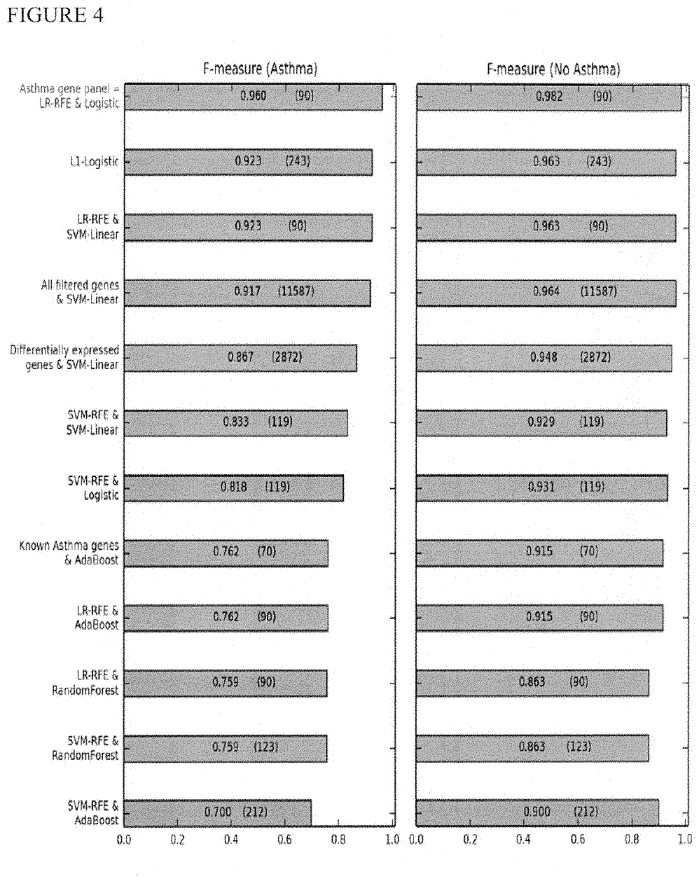

As context for comparison to other models possible from the machine learning pipeline and other methods, FIG. 4 shows the performance of the 90-gene LR-RFE & Logistic model in the test set relative to those of classification models built using (1) other combinations tested in the machine learning pipeline, (2) all genes after filtering (11587 genes), (3) differentially expressed genes (Table 2A-2B), (4) 70 known asthma genes²⁹ (Table 3) and (5) a commonly used one-step classification model (L1-Logistic, 243 genes). All these models performed significantly better than their random counterparts. The LR-RFE & Logistic Model asthma gene panel performed consistently among all the models derived from the machine learning pipeline, as had been expected based on the extensive training and analysis on the development set. The LR-RFE & Logistic Model asthma gene panel also outperformed the model learned using the one-step L1-Logistic method. By separating the feature/gene selection and (outer) classification components, the machine learning pipeline was able to learn a more accurate and more parsimonious classification model, both of which are valuable qualities for disease classification, than L1-Logistic. Overall, these results confirmed that the performance of the LR-RFE & Logistic Model asthma gene panel translated to an independent RNAseq test set, more so than other models, thus lending confidence to this LR-RFE & Logistic Model panel's ability to classify asthma accurately.

Similarly, the other seven classification models and corresponding asthma gene panels performed well in terms of precision and recall, and also beat random performance, such that these models also classify asthma accurately.

Validation of the LR-RFE & Logistic Model Asthma Gene Panel in External Asthma Cohorts

To test the generalizability of the LR-RFE & Logistic Model asthma gene panel for asthma classification, the inventors applied this model to gene expression array data sets generated from two independent cohorts by other investigators with and without asthma (Asthma1GEO GSE19187)³⁰ and Asthma2 (GEO GSE46171)²¹.). Table 5 summarizes the characteristics of these external independent test sets. These datasets were generated from nasal samples collected by independent investigators from subjects with and without asthma from distinct populations, which were then profiled on gene expression microarray platforms. In general, RNA-seq based predictive models are not expected to translate to microarray profiled samples.^{32,33} Gene mappings do not perfectly correspond between RNAseq and microarray due to disparities between array annotations and RNAseq gene models³³. The goal was to assess the performance of the LR-RFE & Logistic Model asthma gene panel despite the discordance of study designs, sample collections, and gene expression profiling platforms.

The inventors found that the LR-RFE & Logistic Model asthma gene panel performed relatively well given the above handicaps, and better than expected in classifying both asthma and no asthma (FIG. 3, middle and right set of bars) and with significantly better performance than permutation-based random models (FIG. 12). In particular, the LR-RFE & Logistic Model asthma gene panel markedly outperformed random models in classifying no asthma in both the Asthma1 and Asthma2 test sets. While classification of asthma in Asthma2 achieved an F-measure of 0.74, its random counterpart also performed well (FIG. 12). Asthma2 included many more asthma cases than controls (23 vs. 5). In such a skewed data set, it is possible for a random model to yield an artificially high F-measure for the majority class (here asthma) by predicting every sample to belong to that class. The inventors verified that this occurred with this random model. These results show that the LR-RFE & Logistic Model asthma gene panel performed reasonably well in these microarray test sets, supporting a degree of generalizability of the panel across platforms and cohorts. Such a translatable result has not been observed very frequently in translational genomic medicine research^{34,35}.

The LR-RFE & Logistic Model Asthma Gene Panel is Specific to Asthma: Validation in External Cohorts with Non-Asthma Respiratory Conditions

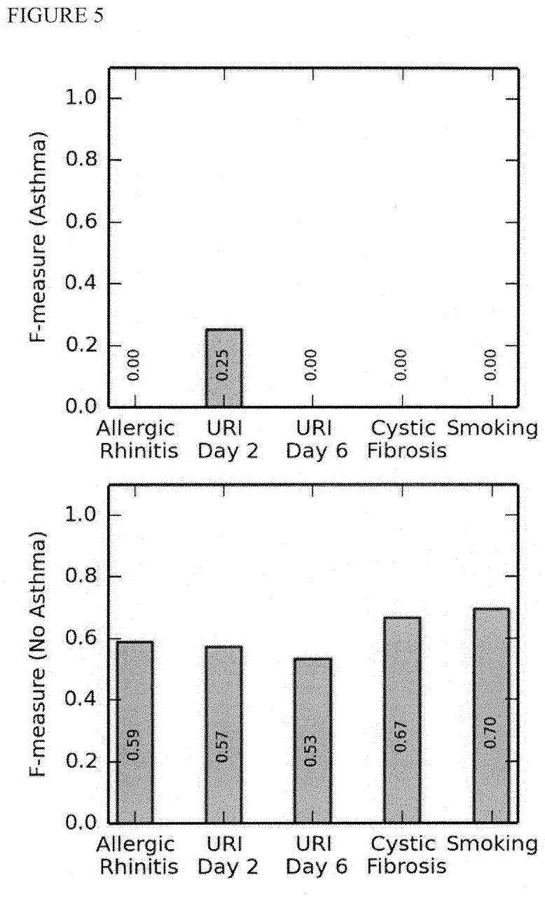

Because symptoms of asthma often overlap with those of other respiratory diseases, the inventors next sought to test the specificity of the LR-RFE & Logistic Model gene panel to asthma classification. For this, the inventors evaluated the performance of this LR-RFE & Logistic Model panel on nasal gene expression data derived from case control cohorts with allergic rhinitis (GSE43523)³⁶, upper respiratory infection (GSE46171)³¹, cystic fibrosis (GSE40445)³⁷, and smoking (GSE8987)¹². Table 6 details the characteristics for these external cohorts with non-asthma respiratory conditions. In four of the five non-asthma data sets, the LR-RFE & Logistic Model asthma gene panel appropriately produced one-sided classifications, i.e., all samples were classified as “no asthma” or healthy, the term for the control class (FIG. 5). Specifically, the positive predictive value of the LR-RFE & Logistic Model panel across these test sets was exactly and appropriately zero for these test sets of non-asthma respiratory conditions (Table 7). The one exception to this was upper respiratory infection (URI2) profiled on day 2 of the illness, where the LR-RFE & Logistic Model panel classified some samples as asthma (F=0.25). This may have been influenced by common inflammatory pathways underlying early viral inflammation and asthma³⁸. Nonetheless, consistent with the other non-asthma test sets, the panel's misclassification of URI2 as asthma was substantially less than its random counterparts (FIG. 13). These results show that the invented method is specific for classifying asthma and would not misclassify other respiratory diseases as asthma.

Examination of Genes in the LR-RFE & Logistic Model Asthma Gene Panel

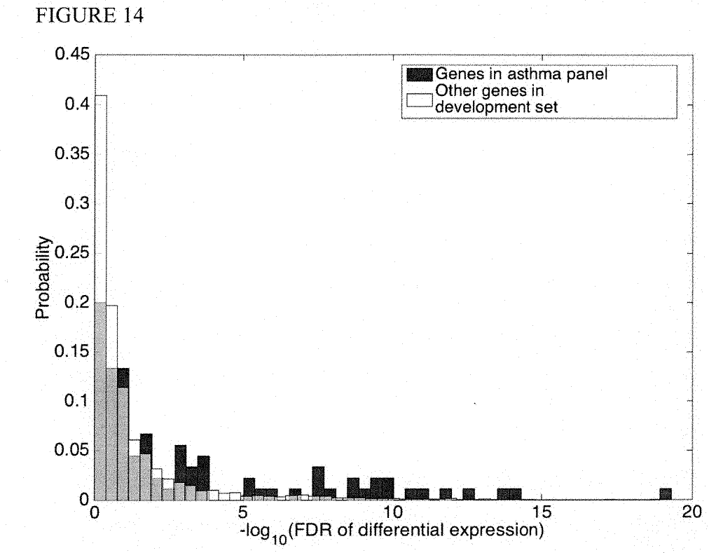

Forty-six of the 90 genes included in the LR-RFE & Logistic Model panel were differentially expressed (FDR≤0.05), with 22 and 24 genes over- and under-expressed in asthma respectively (FIG. 6, Table 2A-2B). More generally, the genes in LR-RFE & Logistic Model panel had lower differential expression FDR values than other genes (Kolmogorov-Smirnov statistic=0.289, P-value=2.73×10−37) (FIG. 14). Pathway enrichment analysis of these 90 genes was statistically limited by the small number of genes, yielding enrichment for pathways including defense response (fold change=2.86, FDR=0.006) and response to external stimulus (fold change=2.50, FDR=0.012). Only four (C3, DEFB1, CYFIP2 and GSTT1) of the 90 genes are known asthma genes and are functionally involved in complement activation, microbicidal activity, T-cell differentiation, and oxidative stress, respectively²⁹. These results suggest that the machine learning pipeline was able to extract information beyond individually differentially expressed or previously known asthma genes, allowing for the identification of a parsimonious panel of genes, including the LR-RFE & Logistic Model panel, that collectively enabled accurate asthma classification.

### Discussion

The inventors have identified a panel of genes, as well as subsets of these genes for use with specific classifiers, expressed in nasal epithelium that accurately classifies subjects with mild/moderate asthma from healthy controls. This asthma gene panel, consisting of 275 unique genes interpreted via eight logistic regression classification models, performed with good precision and sensitivity. Specifically, the LR-RFE & Logistic model and associated asthma gene panel performed with high precision (PPV=1.00 and NPV=0.96) and sensitivity (0.92 and 1.00 for asthma and no asthma respectively) for classifying asthma. The performance of the LR-RFE & Logistic Model asthma gene panel across independent asthma test sets supports the generalizability of this panel across different study populations and two major modalities of gene expression profiling (RNA sequencing and microarray), as well as the specificity of this LR-RFE & Logistic Model panel as a diagnostic tool for asthma in particular, as well as the gene panels identified by the other seven models as discussed herein.

The asthma gene panel has high potential to be used as a minimally invasive biomarker to aid in asthma diagnosis in children and adults, as it can be quickly obtained by simple nasal brush, does not require machinery for collection, and is easily interpreted. According to the Global Initiative for Asthma and US National Heart Lung Blood Institute, the diagnosis of asthma should be based on a history of typical symptoms and objective findings of variable expiratory airflow limitation by PFT^{6, 7}. Practically, however, objective findings are often not obtainable. Patients with mild/moderate asthma are frequently asymptomatic at the time of the clinical encounter, so they may have no detectable wheezing or cough on exam. Pulmonary function testing (PFT) is often not done for patients, as was keenly demonstrated by a study showing that over half of 465,866 patients age 7 years and older with newly diagnosed with asthma had no PFTs performed within a 3.5 year time period surrounding the time of diagnosis.⁸ Clinicians may defer PFTs due to lack of equipment, time, and/or expertise to perform and interpret results^{8, 9}. Diagnosing asthma based on history alone contributes to its under-diagnosis, as patients with asthma under-perceive and under-report their symptoms¹¹. Misdiagnosis of asthma also occurs frequently given overlapping symptoms between asthma and other conditions³⁹. Even if PFTs are obtained, spirometric abnormalities in mild/moderate asthmatics are not always present. An objective, accurate diagnostic tool that is easy and quick to obtain and interpret with minimal effort required by the provider and patient could improve asthma diagnosis so that appropriate management can be pursued. The nasal brush-based asthma gene panel meets these biomarker criteria.

Implementation of the asthma gene panel could involve clinicians brushing a patient's nose, placing the brush in a prepackaged tube, and submitting the sample for gene expression profiling targeted to the panel. Some platforms allow for direct transcriptional profiling of tissue without an RNA isolation step, avoiding inconveniences associated with direct RNA work^{40, 41} and yielding comparable results to RNAseq⁴². Bioinformatic interpretation of the output via the LR-RFE & Logistic model and classification threshold could be automated, resulting in a determination of asthma or no asthma for the clinician to consider. Biomarkers based on gene expression profiling are being successfully used in other disease areas (e.g., MammaPrint⁴³ and Oncotype DX⁴⁴ for diagnosing/predicting breast cancer phenotypes).

Because it takes seconds for nasal brushing, the panel may be attractive to time-strapped clinicians, particularly primary care providers at the frontlines of asthma diagnosis. Asthma is frequently diagnosed and treated in the primary care setting⁴⁵ where access to PFTs is often not immediately available. Although PFTs yield results without specimen handling, these advantages do not seem to overcome its logistical limitations as evidenced by their low rate of real-life implementation, 9 but low cost⁴⁶. However, gene expression profiling costs are likely to decrease47, and implementation of the LR-RFE & Logistic Model asthma gene panel could result in cost savings if it reduces the under-diagnosis and misdiagnosis of asthma³. Undiagnosed asthma leads to costly healthcare utilization worldwide³, including in the United States, where asthma accounts for $56 billion in medical costs, lost school and work days, and early deaths⁴⁸. Clinical implementation of the asthma gene panel could identify undiagnosed asthma, leading to its appropriate management before high healthcare costs from unrecognized asthma are incurred. Given the the LR-RFE & Logistic Model panel's demonstrated specificity, use of the LR-RFE & Logistic Model asthma gene panel could also reduce asthma misdiagnosis by correctly providing a determination of “no asthma” in non-asthmatic subjects with conditions often confused with asthma. Clinical benefit from gene-expression based biomarkers has already been seen in the breast cancer field, where use of the 70-gene panel test MammaPrint to guide chemotherapy in a clinical trial leads to a lower 5-year rate of survival without metastasis compared to standard management⁴³.

The nasal brush-based asthma gene panel capitalizes on the common biology of the upper and lower airway, a concept supported by clinical practice and previous findings.¹²⁻¹⁵ Clinically, clinicians rely on the united airway by screening for lower airway infections (without limitation, influenza, methicillin-resistant Staphylococcus aureus) with nasal swabs.⁴⁹ Sridhar et al. found that gene expression consequences of tobacco smoking in bronchial epithelial cells were reflected in nasal epithelium.¹² Wagener et al. compared gene expression in nasal and bronchial epithelium from 17 subjects, finding that 99.9% of 33,000 genes tested exhibited no differential expression between nasal and bronchial epithelium in those with airway disease.¹³ In a study of 30 children, Guajardo et al. identified gene clusters with differential expression in exacerbated asthma vs. controls.¹⁴ The above studies were done with small sample sizes and microarray technology, although more recently, Poole et al. compared RNA-seq profiles of nasal brushings from 10 asthmatic and 10 control subjects to publically available bronchial transcriptional data, finding strong correlation (ρ=0.87) between nasal and bronchial transcripts, and strong correlation (ρ=0.77) between nasal differential expression and previously observed bronchial differential expression in asthmatics.¹⁵

Although based on only 90 genes, the LR-RFE & Logistic Model asthma gene panel classified asthma with greater accuracy than models using all differentially expressed genes in the sample (n=2187), all known asthma genes from genetic studies of asthma (n=70), as well as models based on information from all sequenced genes (n=11587 after filtering) (FIG. 4). Its superior performance supports that the machine learning pipeline described herein successfully selected a parsimonious set of informative genes that (1) captures more actionable knowledge than those identified by traditional differential expression and genetic analyses, and (2) cuts through the noise of genes that are irrelevant to asthma. The genes selected by the other seven models listed in Table 4 are also highly precise and have good recall. About half the genes in the LR-RFE & Logistic Model asthma gene panel were not differentially expressed at FDR≤0.05, and as such would not have been examined with greater interest if the inventors had performed only differential expression analysis, which is the main analytic approach of virtually all studies of gene expression in asthma.^{12-15, 50, 51} The differential expression FDRs of the 90 genes in the LR-RFE & Logistic Model panel were skewed toward lower values as compared to the rest of the genes in our development set (FIG. 14). This demonstrated that the LR-RFE & Logistic Model asthma gene panel captures signal from differential expression as well as genes below traditional significance thresholds that may still have a contributory role in asthma classification. Only four of the 90 genes in the LR-RFE & Logistic Model gene panel (complement component 3 (C3), defensing beta-1 (DEFB1), cytoplasmic FMR1 interacting protein (CYFIP2) and glutathione S-transferase theta 1 (GSTT1) were genes previously identified by genetic association studies.²⁹In this study, the inventors were able to use the machine learning pipeline to identify this LR-RFE & Logistic Model panel of 90 genes—comprised of both differentially expressed and non-differentially expressed genes, and of genes largely without known genetic associations with asthma—whose gene expression levels can be jointly interpreted via a logistic regression algorithm to accurately predict asthma status.

The asthma gene panel did not perform quite as well in the asthma microarray test sets, and this was to be expected due to differences in study design between the RNAseq and and microarray test sets. First, the baseline characteristics and phenotyping of the subjects differed. Subjects in the RNAseq test set were adults who were classified as mild/moderate asthmatic or healthy using the same strict criteria as the development set (see Materials and Methods above), which required subjects with asthma to have an objective measure of obstructive airway disease (i.e., positive methacholine challenge response). In contrast, subjects in the Asthma1 microarray test set were all children (i.e., not adults) with underlying allergic rhinitis and dust mite allergen 358 sensitivity, whose asthma status was then determined clinically³⁰ (Table 5). Subjects from the Asthma2 cohort were adults who were classified as having asthma or as healthy based on history. As mentioned, the diagnosis of asthma based on history alone without objective lung function testing can be inaccurate⁵². The phenotypic differences between these test sets alone could explain the differences in performance of the LR-RFE & Logistic Model asthma gene panel in the microarray test sets. Second, the differential performance may be due to the difference in gene expression profiling approach. Gene mappings do not perfectly correspond between RNAseq and microarray due to disparities between array annotations and RNAseq gene models.³³ Compared to microarrays, RNAseq quantifies more RNA species and captures a wider range of signal.⁵⁰ Prior studies have shown that microarray-derived models can reliably predict phenotypes based on samples' RNAseq profiles, but the converse does not often hold.³³ Despite the above limitations, the asthma gene panel (identified using the RNAseq-derived development set) performed with reasonable accuracy in classifying asthma in the independent microarray test sets. These results support the generalizability of the asthma gene panel to asthma populations that may be phenotyped or profiled differently.

An effective biomarker for clinical use should have good positive and negative predictive value.⁵³ In the present method, if an individual has asthma, the ideal biomarker would confirm this most of the time so that an accurate diagnosis is made, and if an individual does not have asthma, the ideal biomarker would confirm this (indicating “no asthma”) so that misdiagnosis does not occur. This is indeed the case with the LR-RFE & Logistic Model asthma gene panel, which achieved high positive and negative predictive values of 1.00 and 0.96 respectively on the RNAseq test set. The inventors tested the LR-RFE & Logistic Model asthma gene panel on independent tests sets of subjects with upper respiratory infection, cystic fibrosis, allergic rhinitis, and smoking, showing that the panel had a low to zero rate of misclassifying subjects with these other respiratory conditions as having asthma (FIG. 5). These results were particularly notable for allergic rhinitis, a predominantly nasal condition. Although the asthma gene panel is based on nasal gene expression, and asthma and allergic rhinitis frequently co-occur²³, the LR-RFE & Logistic Model panel did not misdiagnose allergic rhinitis as asthma. These results support the specificity of the LR-RFE & Logistic Model asthma gene panel, as well as the gene panels identified in the other models, as a diagnostic tool for asthma in particular.

Even though the development set was from a single center and its baseline characteristics do not characterize all populations, variancePartition analysis demonstrated minimal contribution of age, race, and gender to gene expression variance in these data (FIG. 7). Further, the LR-RFE & Logistic Model panel performed well in multiple external data sets spanning children and adults of varied racial distributions, and with asthma and other respiratory conditions defined by heterogeneous criteria. Subjects with asthma in the development cohort were not all symptomatic at the time of sampling. The fact that the performance of the LR-RFE & Logistic Model asthma gene panel does not rely on symptomatic asthma is a strength, as many mild/moderate asthmatics are only sporadically symptomatic given the fluctuating nature of the disease.

As with any disease, the first step is to accurately identify affected patients. The asthma gene panel described in this study provides an accurate path to this critical diagnostic step. With a correct diagnosis, an array of existing asthma treatment options can be considered⁶. A next phase of research will be to develop a nasal biomarker to predict endotypes and treatment response, so that asthma treatment can be targeted, and even personalized, with greater efficiency and effectiveness⁵⁴.

In summary, the inventors applied a machine learning pipeline to identify a panel of genes expressed in nasal epithelium that accurately classifies subjects with mild/moderate asthma from healthy controls. This asthma gene panel, comprised of 275 genes and/or its subsets used in combination with model-specific classifiers and model-specific optimal classification thresholds, performed with accuracy across 8 independent test sets, demonstrating generalizability across study populations and gene expression profiling modality, as well as specificity to asthma. The asthma gene panel has high potential to be used as a minimally invasive biomarker to aid in asthma diagnosis, as it can be quickly obtained by simple nasal brush, does not require machinery for collection, and is easily interpreted. There are currently many limitations in asthma diagnostics. If applied to clinical practice, this asthma gene panel could improve asthma diagnosis and classification, reduce incorrect diagnoses, and prompt appropriate therapeutic management.

Table 2. Lists of over-expressed (A) and under-expressed (B) genes and pathways in asthma cases as compared to controls. Differentially expressed genes were identified using DESeq2²⁵ and enriched pathways were identified from the Molecular Signature Database²⁶.

TABLE 2A
Over-expressed Genes and Pathways

| | Fold | |
|---|---|---|
| Gene/Pathway | Change/Description | FDR |
| SDK1 | 2.69593084 | 5.40181E−20 |
| ZDHHC1 | 2.33556546 | 1.23118E−19 |
| SSBP4 | 2.16530278 | 2.57344E−19 |
| C10orf95 | 3.09615627 | 3.8891E−18 |
| ZNF853 | 3.05377899 | 2.25024E−15 |
| PRRT3 | 1.97782866 | 2.40254E−15 |
| ODF3B | 3.0809781 | 3.64261E−15 |
| BZRAP1 | 2.42875066 | 3.96241E−15 |
| HAGHL | 4.04252549 | 7.90746E−15 |
| CROCC | 3.12056593 | 8.21575E−15 |
| C6orf108 | 1.8717848 | 8.86186E−15 |
| PTPRN2 | 2.24409883 | 1.20755E−14 |
| SERPINF1 | 2.03790903 | 1.47636E−14 |
| P4HTM | 2.12086604 | 1.86794E−14 |
| C19orf51 | 4.6822365 | 3.60797E−14 |
| ZSCAN18 | 2.59451449 | 3.60797E−14 |
| B9D2 | 2.07415317 | 3.60797E−14 |
| ARHGAP39 | 2.49865011 | 5.35894E−14 |
| FOXJ1 | 4.26776351 | 5.88781E−14 |
| LRRC10B | 4.42558987 | 6.5261E−14 |
| CCDC42B | 4.2597176 | 6.5261E−14 |
| GAS2L2 | 4.70879795 | 7.82923E−14 |
| C6orf154 | 3.9015674 | 8.44201E−14 |
| GLIS3 | 2.36625326 | 1.00754E−13 |
| LRRC61 | 2.06053632 | 1.09813E−13 |
| ENDOG | 1.97993156 | 1.71162E−13 |
| IRX3 | 1.83337486 | 2.01018E−13 |
| CAPS | 4.06302266 | 2.40086E−13 |
| LPHN1 | 2.10407317 | 2.68055E−13 |
| C2orf55 | 2.27283672 | 3.17873E−13 |
| SYNGAP1 | 2.13301423 | 4.22489E−13 |
| CCDC24 | 1.96494776 | 4.42276E−13 |
| SLC16A11 | 2.0521962 | 4.51489E−13 |
| UCKL1.AS1 | 3.82462625 | 6.69507E−13 |
| RRAD | 3.39266415 | 6.69507E−13 |
| NHLRC4 | 4.55169722 | 7.65957E−13 |
| PRR7 | 2.91887265 | 7.94092E−13 |
| RAB3B | 4.24372545 | 8.15138E−13 |
| CCDC17 | 4.24211711 | 8.23826E−13 |
| ANKRD54 | 2.03165888 | 9.41636E−13 |
| TCTEX1D4 | 4.30165643 | 9.81969E−13 |
| PPP1R16A | 1.78187416 | 1.01874E−12 |
| NAT14 | 3.06261532 | 1.03487E−12 |
| CTXN1 | 4.61823126 | 1.03958E−12 |
| ANKK1 | 2.06364461 | 1.03958E−12 |
| MAPK15 | 4.61083061 | 1.07813E−12 |
| TEKT2 | 4.78797511 | 1.13157E−12 |
| CCDC96 | 2.89251884 | 1.13157E−12 |
| CXCR7 | 2.57340048 | 1.18772E−12 |
| SPEF1 | 4.04138282 | 1.28995E−12 |
| C2orf81 | 3.88312294 | 1.62387E−12 |
| TPPP3 | 4.1122218 | 1.95083E−12 |
| TP73 | 3.73216045 | 2.05602E−12 |
| C17orf72 | 4.12597857 | 2.42931E−12 |
| KIF19 | 4.04831578 | 2.42931E−12 |
| CRNDE | 1.90266433 | 2.42931E−12 |
| FDXR | 1.75411331 | 2.42931E−12 |
| TNFAIP8L1 | 3.66812001 | 2.52964E−12 |
| IFT140 | 2.56011824 | 2.52964E−12 |
| FBXW9 | 2.0309423 | 3.71669E−12 |
| ESPN | 1.78254716 | 4.12128E−12 |
| DFNB31 | 1.8555535 | 4.1682E−12 |
| TTLL10 | 3.97446989 | 4.96622E−12 |
| FAM116B | 2.76115746 | 5.75046E−12 |
| CCDC19 | 3.97176187 | 5.83187E−12 |
| C6orf27 | 3.15382185 | 6.10565E−12 |
| C16orf48 | 2.28318997 | 6.26965E−12 |
| GAS8 | 1.96553042 | 6.26965E−12 |
| CD164L2 | 3.21331723 | 6.36707E−12 |
| CCDC78 | 4.79072783 | 6.85549E−12 |
| CCDC40 | 4.02185553 | 7.85218E−12 |
| CCDC157 | 2.50320674 | 1.03363E−11 |
| UBXN11 | 2.67485867 | 1.12753E−11 |
| C9orf24 | 4.24049927 | 1.13692E−11 |
| B9D1 | 2.93782564 | 1.3303E−11 |
| LRRC56 | 2.57381093 | 1.60583E−11 |
| PKIG | 2.47239105 | 1.60583E−11 |
| ADSSL1 | 1.963967 | 1.70739E−11 |
| PASK | 2.00442189 | 1.93192E−11 |
| C5orf49 | 3.85710623 | 1.95595E−11 |
| TUBB2C | 2.04908703 | 2.17307E−11 |
| HSPBP1 | 1.8050605 | 2.17307E−11 |
| DLEC1 | 4.80156726 | 2.39955E−11 |
| ANKMY1 | 2.5681388 | 2.39955E−11 |
| RUVBL2 | 1.8875842 | 2.41852E−11 |
| WDR54 | 3.54079973 | 2.48129E−11 |
| CCDC108 | 4.40594345 | 2.82076E−11 |
| USP2 | 2.61579764 | 2.82076E−11 |
| WDR90 | 2.25341462 | 3.47445E−11 |
| SLC1A4 | 1.7743007 | 3.60414E−11 |
| ISYNA1 | 1.78188864 | 3.90247E−11 |
| LRRC48 | 4.23655785 | 4.33546E−11 |
| SLC27A2 | 1.77294486 | 4.33546E−11 |
| C11orf16 | 4.16123887 | 4.35926E−11 |
| BBS5 | 2.05305886 | 4.96429E−11 |
| C14orf79 | 1.9431267 | 4.96429E−11 |
| DNAAF2 | 1.82683937 | 5.32802E−11 |
| IQCD | 2.99396253 | 5.9179E−11 |
| PPOX | 2.466844 | 5.9179E−11 |
| ZNF703 | 1.80994279 | 6.27934E−11 |
| IGFBP2 | 2.12208723 | 6.3397E−11 |
| KCNH3 | 3.74731532 | 6.67127E−11 |
| RHPN1 | 2.11269443 | 6.74204E−11 |
| KNDC1 | 4.27320927 | 8.33894E−11 |
| TRAF3IP1 | 1.80219185 | 8.80362E−11 |
| FAM92B | 3.96288061 | 8.91087E−11 |
| C5orf4 | 2.02530771 | 9.38443E−11 |
| MAP6 | 4.48787026 | 9.67629E−11 |
| IQCE | 1.88795828 | 9.71132E−11 |
| INPP5E | 1.8396103 | 9.71132E−11 |
| NWD1 | 3.99394282 | 1.13238E−10 |
| DNAH9 | 4.39061797 | 1.16455E−10 |
| LTBP3 | 1.62487623 | 1.3309E−10 |
| CDK20 | 2.3240984 | 1.54953E−10 |
| CCNO | 2.32391131 | 1.55262E−10 |
| RAB36 | 3.80755493 | 1.59581E−10 |
| WDR34 | 1.87639055 | 1.87132E−10 |
| DNAI1 | 4.84949642 | 2.12635E−10 |
| DNAAF1 | 3.83746993 | 2.14037E−10 |
| CCDC164 | 4.2557065 | 2.20169E−10 |
| ASCL2 | 2.04147055 | 2.26234E−10 |
| FHAD1 | 3.13964638 | 2.37682E−10 |
| FAM179A | 4.66078913 | 2.37965E−10 |
| TEKT1 | 4.13606595 | 2.48284E−10 |
| DALRD3 | 1.75343551 | 2.48284E−10 |
| TMCC2 | 1.90615943 | 2.60427E−10 |
| CCDC114 | 4.09401076 | 2.95477E−10 |
| LRWD1 | 1.98021375 | 3.02767E−10 |
| NCRNA00094 | 2.12505456 | 3.12538E−10 |
| WDR38 | 4.23621789 | 3.26822E−10 |
| ALDH3B1 | 1.6813904 | 3.28037E−10 |
| TMEM190 | 4.8685534 | 3.30569E−10 |
| ULK4 | 2.32420099 | 3.48495E−10 |
| DMRT2 | 1.82662574 | 3.48718E−10 |
| C9orf171 | 3.97704489 | 3.72441E−10 |
| FUZ | 2.72661607 | 3.81064E−10 |
| VWA3A | 4.21877596 | 4.49516E−10 |
| CDHR4 | 5.12021012 | 4.57757E−10 |
| METRN | 2.25309804 | 4.57757E−10 |
| LOC113230 | 1.81478964 | 4.57757E−10 |
| DNAI2 | 4.03796529 | 4.76126E−10 |
| TCTN2 | 2.40490432 | 4.95937E−10 |
| FAM166B | 3.90791018 | 5.63709E−10 |
| ZMYND10 | 3.69143549 | 6.00928E−10 |
| MZF1 | 1.76527865 | 6.58326E−10 |
| ROPN1L | 3.43290481 | 6.64612E−10 |
| APBB1 | 2.62366455 | 6.64612E−10 |
| PLEKHB1 | 3.4214872 | 6.72995E−10 |
| LRRC23 | 3.23420407 | 7.30088E−10 |
| SLC4A8 | 3.06635647 | 8.20469E−10 |
| WNT9A | 1.97501893 | 8.98004E−10 |
| CCDC103 | 3.21531173 | 9.17894E−10 |
| C20orf85 | 3.7643551 | 9.37355E−10 |
| TSNAXIP1 | 3.67477124 | 9.47472E−10 |
| DNAH2 | 3.69841798 | 9.84984E−10 |
| ZNF474 | 3.52004876 | 1.11372E−09 |
| TPPP | 2.28275479 | 1.11372E−09 |
| TMEM231 | 3.16472296 | 1.12292E−09 |
| TTC12 | 1.91008892 | 1.13249E−09 |
| LDLRAD1 | 3.56956748 | 1.15526E−09 |
| CHCHD10 | 1.87337748 | 1.18307E−09 |
| RFX2 | 2.66731378 | 1.23139E−09 |
| UBXN10 | 3.25532613 | 1.26161E−09 |
| IFT172 | 2.64104339 | 1.3631E−09 |
| BAIAP3 | 3.63613461 | 1.411E−09 |
| EFCAB2 | 2.69292361 | 1.42619E−09 |
| C11orf88 | 3.52355279 | 1.4444E−09 |
| SLC13A3 | 2.20805923 | 1.4444E−09 |
| IFT122 | 2.04426301 | 1.48429E−09 |
| NPHP4 | 1.89172058 | 1.51209E−09 |
| TXNDC5 | 1.86619199 | 1.515E−09 |
| C17orf97 | 2.35986311 | 1.62066E−09 |
| WDR16 | 4.36651228 | 1.62402E−09 |
| DNALI1 | 3.46070328 | 1.63511E−09 |
| NUDT3 | 1.73970966 | 1.64286E−09 |
| SMYD2 | 2.10344741 | 1.70609E−09 |
| TTC25 | 3.71446639 | 2.05596E−09 |
| RBM38 | 1.61948356 | 2.1203E−09 |
| GGT7 | 1.66897144 | 2.14547E−09 |
| CES1 | 3.00060938 | 2.23456E−09 |
| C21orf59 | 1.72965503 | 2.26356E−09 |
| CCDC65 | 3.41519122 | 2.38892E−09 |
| WDR60 | 1.90360794 | 2.48798E−09 |
| UNC119B | 1.68295738 | 2.7675E−09 |
| EML1 | 3.14662458 | 2.86572E−09 |
| ODF2 | 1.77285642 | 2.88517E−09 |
| C20orf96 | 3.28661501 | 2.92408E−09 |
| C21orf2 | 1.59981088 | 2.95269E−09 |
| LRRC45 | 1.73562887 | 2.9555E−09 |
| LOC100506668 | 2.17031169 | 3.52531E−09 |
| GLB1L | 2.06829337 | 3.65952E−09 |
| CCDC74A | 3.2798251 | 3.94098E−09 |
| ABCA2 | 1.64595295 | 3.94098E−09 |
| MAP1A | 3.30677387 | 4.49644E−09 |
| C9orf9 | 3.3529991 | 4.60478E−09 |
| CHST9 | 1.75966672 | 4.8617E−09 |
| MAPRE3 | 2.07180681 | 5.32347E−09 |
| RND2 | 2.18107852 | 5.44526E−09 |
| DGCR6 | 1.8288164 | 5.45688E−09 |
| SNED1 | 1.88272394 | 5.83476E−09 |
| LRRC46 | 4.00288588 | 5.87568E−09 |
| C16orf71 | 3.78067833 | 5.87568E−09 |
| FBXO36 | 1.97697195 | 5.87808E−09 |
| STK33 | 3.32049025 | 5.97395E−09 |
| FANK1 | 3.09673143 | 6.34411E−09 |
| IRF2BPL | 1.5943287 | 6.45821E−09 |
| MEX3D | 1.59132125 | 6.57088E−09 |
| TTC29 | 3.77710968 | 7.14688E−09 |
| SPAG17 | 4.10266721 | 7.18248E−09 |
| DNAH10 | 4.05401954 | 7.37766E−09 |
| C19orf55 | 1.81580403 | 7.5128E−09 |
| GNA14 | 2.3089692 | 7.76554E−09 |
| GPR162 | 3.42624459 | 7.78437E−09 |
| KIF24 | 2.6517961 | 8.23367E−09 |
| C6orf97 | 3.05579163 | 8.66959E−09 |
| ATP2C2 | 1.60268251 | 8.79826E−09 |
| EFHC1 | 3.13154257 | 1.00071E−08 |
| C9orf1l6 | 2.98680162 | 1.02805E−08 |
| TUBA4B | 3.44329925 | 1.10115E−08 |
| TUB | 3.28725084 | 1.10581E−08 |
| IGFBP5 | 3.42171001 | 1.12425E−08 |
| GOLGA2B | 1.87746797 | 1.15371E−08 |
| RAGE | 2.48773652 | 1.16413E−08 |
| UCP2 | 1.52039355 | 1.17729E−08 |
| KIAA1407 | 2.63617454 | 1.18646E−08 |
| TTC21A | 2.5095734 | 1.20361E−08 |
| C1orf173 | 3.85335748 | 1.24014E−08 |
| PSENEN | 1.74442606 | 1.26734E−08 |
| MAPK8IP1 | 2.43031719 | 1.31409E−08 |
| WDR52 | 2.7867767 | 1.3227E−08 |
| RCAN3 | 1.67977331 | 1.32982E−08 |
| REC8 | 2.71104704 | 1.35783E−08 |
| KCTD1 | 1.63948363 | 1.35783E−08 |
| ZNF579 | 1.56261805 | 1.43116E−08 |
| NCALD | 2.31903784 | 1.48365E−08 |
| IFT43 | 1.8372634 | 1.6037E−08 |
| GALNS | 1.69455658 | 1.60813E−08 |
| RABL5 | 2.20299003 | 1.6314E−08 |
| SLC22A4 | 2.22553299 | 1.66879E−08 |
| CC2D2A | 3.16499889 | 1.70886E−08 |
| C12orf75 | 2.65337293 | 1.74645E−08 |
| MS4A8B | 4.57793875 | 1.78335E−08 |
| DNAH5 | 3.74507278 | 1.82168E−08 |
| LRTOMT | 2.78785677 | 1.91101E−08 |
| C18orf1 | 1.87715316 | 1.91101E−08 |
| TRADD | 1.56913276 | 1.97067E−08 |
| C1orf194 | 3.88158651 | 1.98158E−08 |
| STOX1 | 2.81737017 | 2.04397E−08 |
| SPAG6 | 3.38226503 | 2.05137E−08 |
| EFCAB6 | 3.13972956 | 2.0547E−08 |
| CDHR3 | 4.50496815 | 2.09665E−08 |
| C1orf192 | 3.27606806 | 2.13713E−08 |
| ST6GALNAC2 | 1.69322433 | 2.13713E−08 |
| CEP250 | 1.63128892 | 2.13713E−08 |
| RSPH9 | 3.5289842 | 2.2596E−08 |
| RFX3 | 2.64245161 | 2.28181E−08 |
| DMRTA2 | 1.55534501 | 2.28181E−08 |
| CCDC113 | 3.00709138 | 2.33952E−08 |
| TCTN1 | 2.57027348 | 2.43901E−08 |
| ZNHIT2 | 1.68919209 | 2.59867E−08 |
| NELL2 | 4.27702275 | 2.62282E−08 |
| DNAH3 | 3.76161641 | 2.68229E−08 |
| RSPH1 | 3.9078246 | 2.79364E−08 |
| IPO4 | 1.62195554 | 2.83731E−08 |
| OSBPL6 | 2.51046395 | 2.86967E−08 |
| NPHP1 | 3.03497793 | 2.87686E−08 |
| NPEPL1 | 1.80587307 | 2.93319E−08 |
| PCDP1 | 3.86414265 | 3.03499E−08 |
| HES6 | 2.83951527 | 3.03499E−08 |
| OSCP1 | 2.46419674 | 3.16173E−08 |
| C6orf225 | 2.88981515 | 3.16232E−08 |
| RDH14 | 1.85367299 | 3.20457E−08 |
| WDR31 | 1.86799234 | 3.3187E−08 |
| NRSN2 | 1.72859689 | 3.33598E−08 |
| CYB5D1 | 2.01628245 | 3.53966E−08 |
| FAAH | 1.64399385 | 3.56421E−08 |
| LRRC27 | 1.81134305 | 3.62992E−08 |
| CIB1 | 1.51834252 | 3.65446E−08 |
| SPPL2B | 1.52835317 | 3.68019E−08 |
| CROCCP2 | 1.60146337 | 3.69799E−08 |
| NFIX | 1.57340231 | 3.71894E−08 |
| RIBC1 | 3.0954211 | 3.73058E−08 |
| ARMC2 | 2.45822891 | 3.73058E−08 |
| KIF9 | 2.3180051 | 3.79512E−08 |
| COQ4 | 1.56458854 | 3.96258E−08 |
| WDR66 | 3.18527022 | 4.13597E−08 |
| KLHL6 | 3.05051676 | 4.13597E−08 |
| ANKRD9 | 1.68315489 | 4.18769E−08 |
| PPIL6 | 3.49881233 | 4.5818E−08 |
| CELSR1 | 1.5798801 | 4.61481E−08 |
| ECT2L | 3.92659277 | 4.67195E−08 |
| TMEM107 | 2.25606657 | 4.72838E−08 |
| IL5RA | 3.38598476 | 4.91414E−08 |
| SPATA18 | 3.04142002 | 5.0583E−08 |
| ZNF865 | 1.55350931 | 5.11875E−08 |
| MKS1 | 1.72625587 | 5.31129E−08 |
| DNAH12 | 4.07123221 | 5.46701E−08 |
| SNTN | 3.41828613 | 5.48011E−08 |
| SNAPC4 | 1.55079316 | 5.48488E−08 |
| KLHDC9 | 2.21375808 | 5.68972E−08 |
| MTSS1 | 1.59589799 | 5.76209E−08 |
| PTRH1 | 1.64149801 | 5.78872E−08 |
| C16orf55 | 2.03868071 | 5.8729E−08 |
| C7orf57 | 3.24294862 | 6.00827E−08 |
| NUDC | 1.54151756 | 6.10697E−08 |
| TNFRSF19 | 2.20738343 | 6.27622E−08 |
| IQCG | 2.95680296 | 6.2973E−08 |
| VWA3B | 3.70172326 | 6.30683E−08 |
| KAL1 | 2.86964004 | 6.30683E−08 |
| WRAP53 | 1.93108611 | 6.30683E−08 |
| CLUAP1 | 1.88649708 | 6.34659E−08 |
| PACRG | 3.25262251 | 6.37979E−08 |
| CCDC81 | 3.4942349 | 6.42368E−08 |
| AKR7A2 | 1.57742473 | 6.47208E−08 |
| KCNE1 | 3.35236141 | 6.58782E−08 |
| INHBB | 3.2633604 | 6.79537E−08 |
| PRDX5 | 1.55465969 | 6.79537E−08 |
| MYB | 1.84122844 | 6.81621E−08 |
| NEK11 | 2.74190303 | 6.81892E−08 |
| RUVBL1 | 2.00081999 | 6.99548E−08 |
| SYNE1 | 2.93233229 | 7.1936E−08 |
| C17orf79 | 1.59608063 | 7.31685E−08 |
| JAG2 | 2.00848549 | 7.85574E−08 |
| ACOT2 | 1.61704514 | 8.52356E−08 |
| PRSS12 | 1.60068977 | 8.62009E−08 |
| PHGDH | 2.07652258 | 8.78686E−08 |
| AK8 | 2.99751993 | 8.85495E−08 |
| C11orf49 | 1.65594025 | 8.87426E−08 |
| SYT5 | 3.23619723 | 9.00219E−08 |
| C3orf15 | 3.55197982 | 9.33003E−08 |
| PAX3 | 1.68131102 | 9.48619E−08 |
| SHANK2 | 3.08586078 | 9.57305E−08 |
| AK7 | 3.11167056 | 1.04568E−07 |
| DIXDC1 | 2.20355836 | 1.04568E−07 |
| ACCN2 | 1.63822574 | 1.04568E−07 |
| TBX1 | 1.62839701 | 1.05101E−07 |
| HYDIN | 3.64358909 | 1.0567E−07 |
| C13orf30 | 3.57465645 | 1.06437E−07 |
| ANKRD37 | 2.08781744 | 1.06496E−07 |
| POMT2 | 1.77671355 | 1.06496E−07 |
| C21orf58 | 3.15402189 | 1.14416E−07 |
| CNTRL | 1.98315627 | 1.15119E−07 |
| SIX2 | 1.56975674 | 1.16144E−07 |
| GLB1L2 | 1.87516329 | 1.18115E−07 |
| ZNF440 | 1.62497497 | 1.18115E−07 |
| SYTL3 | 1.60669405 | 1.18115E−07 |
| ERCC1 | 1.55757069 | 1.18115E−07 |
| DNAH1 | 2.22541262 | 1.18941E−07 |
| FAM154B | 3.2374058 | 1.20444E−07 |
| EFCAB1 | 3.41783606 | 1.24931E−07 |
| BBS1 | 1.62663444 | 1.26292E−07 |
| PRUNE2 | 3.09870519 | 1.26484E−07 |
| H1FX | 1.54347559 | 1.26484E−07 |
| IFT57 | 2.02384988 | 1.27781E−07 |
| ARMC3 | 3.6866857 | 1.28185E−07 |
| C1orf201 | 1.97130635 | 1.32673E−07 |
| C20orf12 | 2.16851256 | 1.35408E−07 |
| FAM183A | 3.43889722 | 1.35507E−07 |
| ZBBX | 3.75926958 | 1.37771E−07 |
| C1orf88 | 3.33179192 | 1.44064E−07 |
| EFHB | 3.24198197 | 1.45387E−07 |
| YSK4 | 3.13700382 | 1.50138E−07 |
| CCDC60 | 2.03255306 | 1.50341E−07 |
| TUSC3 | 1.69381639 | 1.50981E−07 |
| CES4A | 2.40159419 | 1.51353E−07 |
| CAP2 | 2.30419698 | 1.5299E−07 |
| STOML3 | 3.56916735 | 1.54086E−07 |
| PCYT2 | 1.54216983 | 1.61706E−07 |
| SLFN13 | 2.24221791 | 1.6531E−07 |
| DNAL4 | 1.73946873 | 1.6531E−07 |
| C2CD2L | 1.53455465 | 1.65577E−07 |
| IFT46 | 1.9344197 | 1.7083E−07 |
| DNAH6 | 3.67492559 | 1.74274E−07 |
| RSPH4A | 3.32798921 | 1.74274E−07 |
| DTHD1 | 3.32521784 | 1.74542E−07 |
| SLC12A7 | 1.58126148 | 1.7563E−07 |
| DPCD | 1.93856115 | 1.76542E−07 |
| DNAH7 | 3.36255762 | 1.78119E−07 |
| NTN1 | 1.52761436 | 1.78206E−07 |
| CLDN3 | 1.84043179 | 1.8233E−07 |
| RHOBTB1 | 1.75019548 | 1.87553E−07 |
| APOBEC4 | 3.28732642 | 1.8767E−07 |
| FAM174A | 1.51418232 | 1.90288E−07 |
| ARMC9 | 1.90867648 | 1.91275E−07 |
| PLTP | 1.60313361 | 1.98108E−07 |
| CCDC146 | 2.6710312 | 2.0177E−07 |
| C14orf45 | 2.54462539 | 2.13129E−07 |
| OBSCN | 1.86629325 | 2.1622E−07 |
| WDR96 | 4.51826736 | 2.1911E−07 |
| SFXN3 | 1.59966258 | 2.19516E−07 |
| GALM | 1.59756388 | 2.19516E−07 |
| FAM81B | 3.17612876 | 2.22082E−07 |
| EFEMP2 | 1.61941953 | 2.24048E−07 |
| RABL2A | 2.30603938 | 2.28887E−07 |
| WDR78 | 3.09268044 | 2.33992E−07 |
| C10orf107 | 3.16756032 | 2.44725E−07 |
| C9orf135 | 2.86769508 | 2.44725E−07 |
| NEURL1B | 2.13311341 | 2.44782E−07 |
| BCAM | 2.0015908 | 2.44782E−07 |
| PKD1 | 1.53249813 | 2.46006E−07 |
| FBRSL1 | 1.50952964 | 2.46006E−07 |
| DNAJA4 | 1.55609308 | 2.5244E−07 |
| C11orf63 | 2.22050183 | 2.53161E−07 |
| MAGIX | 1.61223309 | 2.64993E−07 |
| CLMN | 2.07549994 | 2.87911E−07 |
| TNS1 | 1.77612203 | 3.08503E−07 |
| SPA17 | 2.66711922 | 3.17135E−07 |
| CRY2 | 1.54310386 | 3.48954E−07 |
| IQCA1 | 2.54545108 | 3.85583E−07 |
| IFT27 | 2.00349955 | 3.85583E−07 |
| C6orf165 | 3.3160697 | 3.90768E−07 |
| SPATA6 | 1.86634548 | 3.91415E−07 |
| ARMC4 | 3.33542089 | 4.12418E−07 |
| MNS1 | 2.96005772 | 4.20421E−07 |
| AP2B1 | 1.82011977 | 4.27029E−07 |
| ABHD12B | 1.65078768 | 4.58254E−07 |
| RABL2B | 2.18769571 | 4.60153E−07 |
| DNAH11 | 3.39839639 | 4.78493E−07 |
| TCTEX1D2 | 2.32862285 | 4.92481E−07 |
| SNCAIP | 2.15177999 | 5.25094E−07 |
| PRR15 | 1.52053242 | 5.39026E−07 |
| TRAPPC9 | 1.49825676 | 5.47471E−07 |
| C11orf70 | 3.19682649 | 5.52587E−07 |
| MTSS1L | 1.51447468 | 5.77745E−07 |
| IQCC | 1.76671873 | 5.85222E−07 |
| MIPEP | 1.60770446 | 5.87639E−07 |
| CAPSL | 3.22810829 | 6.13092E−07 |
| FBXO31 | 1.52038127 | 6.15582E−07 |
| IGFBP7 | 3.46134083 | 6.47155E−07 |
| GLTSCR2 | 1.39112797 | 6.63441E−07 |
| CASC1 | 2.94972846 | 7.41883E−07 |
| AKAP6 | 2.21859968 | 7.65044E−07 |
| CDC14A | 1.71863036 | 7.65644E−07 |
| GPR172B | 1.68332351 | 7.75027E−07 |
| KIF3B | 1.53993685 | 8.08875E−07 |
| NSUN7 | 1.55243313 | 8.71403E−07 |
| CBY1 | 1.69853505 | 9.10803E−07 |
| MORN2 | 2.28391481 | 9.392E−07 |
| FAM134B | 2.02733713 | 9.45965E−07 |
| LRRIQ1 | 3.26113554 | 9.58549E−07 |
| ZNF446 | 1.52395776 | 9.58549E−07 |
| TTC26 | 2.53343738 | 9.80114E−07 |
| CALML4 | 1.62740933 | 9.95113E−07 |
| LRP11 | 1.49024896 | 1.02382E−06 |
| TMPRSS3 | 1.80633832 | 1.04835E−06 |
| MDM1 | 1.71360038 | 1.07116E−06 |
| PAQR4 | 1.56647668 | 1.16048E−06 |
| SEMA5A | 1.65992081 | 1.18574E−06 |
| IDH2 | 1.48906176 | 1.22485E−06 |
| SLC2A4RG | 1.473539 | 1.28937E−06 |
| WDR27 | 1.86298354 | 1.29757E−06 |
| MB | 1.56393059 | 1.35535E−06 |
| PLCH1 | 2.31329264 | 1.36675E−06 |
| FOXN4 | 2.43309713 | 1.49276E−06 |
| CETN2 | 2.31001093 | 1.51913E−06 |
| ECI1 | 1.46030427 | 1.63719E−06 |
| ACOT1 | 1.71878182 | 1.65012E−06 |
| SPEF2 | 3.00394567 | 1.69058E−06 |
| ENKUR | 3.17038628 | 1.69235E−06 |
| ANKRD42 | 1.7433919 | 1.70496E−06 |
| CSMD1 | 2.01483263 | 1.71638E−06 |
| LRRC49 | 2.42707576 | 1.81419E−06 |
| LRRC6 | 2.41771576 | 2.0278E−06 |
| PDF | 1.72789067 | 2.0278E−06 |
| AP3M2 | 1.6599425 | 2.0278E−06 |
| ATP6V0E2 | 1.51739952 | 2.23414E−06 |
| CYBASC3 | 1.47190218 | 2.47918E−06 |
| MGC2752 | 1.51302987 | 2.49691E−06 |
| CTGF | 2.44083959 | 2.53147E−06 |
| NME7 | 2.30993461 | 2.56434E−06 |
| ICA1L | 1.87405521 | 2.59186E−06 |
| KIAA1377 | 2.35492722 | 2.63213E−06 |
| WNT4 | 1.62388727 | 2.66608E−06 |
| CCDC66 | 1.78966672 | 2.69319E−06 |
| DMD | 1.60710731 | 2.70822E−06 |
| RGMA | 1.77597556 | 2.76587E−06 |
| BCL7A | 1.54768303 | 2.79246E−06 |
| ARL3 | 1.52985757 | 2.88426E−06 |
| FKRP | 1.59965333 | 3.01403E−06 |
| RORC | 1.52931081 | 3.01403E−06 |
| ULK2 | 1.59698142 | 3.04102E−06 |
| ACSS1 | 1.55253699 | 3.07996E−06 |
| HHAT | 1.60739942 | 3.08587E−06 |
| EFNB3 | 2.4297676 | 3.45813E−06 |
| B3GNT9 | 1.55740701 | 3.51732E−06 |
| SLC25A4 | 1.49801843 | 3.55964E−06 |
| CCDC138 | 1.80406427 | 3.56785E−06 |
| PABPN1 | 1.44608578 | 3.69532E−06 |
| SMPD2 | 1.47546999 | 3.70938E−06 |
| ZNF580 | 1.47324953 | 3.73581E−06 |
| OLFML2A | 1.68087252 | 3.7554E−06 |
| C7orf50 | 1.44237361 | 3.94008E−06 |
| LEPREL2 | 1.95758996 | 3.94011E−06 |
| DZIP3 | 2.22081454 | 4.02528E−06 |
| NCRNA00287 | 1.69130571 | 4.03026E−06 |
| C3orf67 | 1.72190896 | 4.09892E−06 |
| IL17RE | 1.48542123 | 4.16438E−06 |
| DUSP18 | 1.76643191 | 4.2E−06 |
| HEATR2 | 1.53592007 | 4.2E−06 |
| CERS4 | 1.46651735 | 4.55413E−06 |
| EFHC2 | 2.54152611 | 4.67467E−06 |
| EBF4 | 1.50785283 | 4.71457E−06 |
| SCAMP4 | 1.44146628 | 4.91032E−06 |
| HEY1 | 1.51597477 | 5.00328E−06 |
| CSPP1 | 2.05160927 | 5.01668E−06 |
| NCS1 | 1.53990962 | 5.02214E−06 |
| ZNF837 | 1.67092737 | 5.22131E−06 |
| CCDC104 | 1.59507824 | 5.28987E−06 |
| DNAL1 | 1.92925734 | 5.86073E−06 |
| TTC38 | 1.47562236 | 5.88772E−06 |
| KIF27 | 2.05357283 | 6.13829E−06 |
| THRA | 1.49828801 | 6.16885E−06 |
| GNAL | 1.51789304 | 6.24393E−06 |
| LCA5 | 2.05878538 | 6.76347E−06 |
| IDAS | 1.71281695 | 7.04626E−06 |
| KIAA0556 | 1.48330058 | 7.50539E−06 |
| PYCR2 | 1.49939954 | 7.88147E−06 |
| TRPV4 | 1.47758825 | 7.88147E−06 |
| TMEM98 | 1.46244012 | 8.21506E−06 |
| DYRK1B | 1.445023 | 8.35968E−06 |
| MEGF8 | 1.4698702 | 8.57212E−06 |
| FAM149 | 1.61900561 | 8.90473E−06 |
| FTO | 1.54233263 | 9.20995E−06 |
| RBKS | 1.66266555 | 9.25498E−06 |
| ORAI3 | 1.46516304 | 9.45553E−06 |
| NDUFAF3 | 1.44305183 | 9.66172E−06 |
| C16orf80 | 1.53411506 | 1.07805E−05 |
| CCDC34 | 1.95285314 | 1.08031E−05 |
| FAM104B | 1.64584961 | 1.08935E−05 |
| NME5 | 2.35890292 | 1.0967E−05 |
| SRGAP3 | 1.51025268 | 1.10599E−05 |
| ALMS1 | 1.75968611 | 1.10615E−05 |
| COL9A2 | 1.46064849 | 1.10777E−05 |
| CNTNAP3 | 1.64650311 | 1.11243E−05 |
| HDAC10 | 1.43909133 | 1.12656E−05 |
| WDR35 | 1.79775411 | 1.18311E−05 |
| PRR12 | 1.44830825 | 1.24302E−05 |
| SNX29 | 1.49309166 | 1.25697E−05 |
| CRIP1 | 2.21165686 | 1.25722E−05 |
| SOBP | 1.70952245 | 1.29589E−05 |
| SLC9A3R2 | 1.38857255 | 1.31279E−05 |
| PHC1 | 1.60359663 | 1.38781E−05 |
| PKN1 | 1.44709171 | 1.38781E−05 |
| TRIP13 | 2.13571915 | 1.40793E−05 |
| SPAG16 | 1.5476954 | 1.41052E−05 |
| TBC1D8 | 1.64734934 | 1.44514E−05 |
| METTL7A | 1.54943803 | 1.45491E−05 |
| NPM2 | 1.64770549 | 1.49453E−05 |
| TSGA14 | 1.83369437 | 1.53621E−05 |
| ABCA3 | 1.56393698 | 1.53948E−05 |
| EPB41L4B | 1.46546865 | 1.55092E−05 |
| SCGB2A1 | 1.85264034 | 1.58836E−05 |
| WDR69 | 3.13080652 | 1.59712E−05 |
| MCAT | 1.44452413 | 1.59712E−05 |
| HSPG2 | 1.44631976 | 1.69312E−05 |
| LRRC26 | 1.74351209 | 1.73709E−05 |
| KIAA0195 | 1.42018377 | 1.73709E−05 |
| RFX1 | 1.41884581 | 1.80687E−05 |
| WDR19 | 1.89888711 | 1.82737E−05 |
| ANKRD35 | 1.4184045 | 1.89416E−05 |
| BBS9 | 1.59591845 | 1.90715E−05 |
| CCDC41 | 1.73056217 | 1.92145E−05 |
| FARP1 | 1.43058432 | 1.92684E−05 |
| NGRN | 1.41426222 | 1.93043E−05 |
| DCAKD | 1.5245559 | 2.01031E−05 |
| KATNAL2 | 1.83549945 | 2.03357E−05 |
| AUTS2 | 1.44446141 | 2.10708E−05 |
| SLC7A2 | 2.78449202 | 2.13078E−05 |
| ZDHHC24 | 1.41648471 | 2.14062E−05 |
| SLC41A1 | 1.52318986 | 2.14929E−05 |
| C8orf47 | 1.59908668 | 2.15109E−05 |
| SHROOM3 | 1.49391839 | 2.15542E−05 |
| SUV420H2 | 1.47743036 | 2.17189E−05 |
| TMEM132A | 1.3601549 | 2.17189E−05 |
| CITED4 | 1.54649834 | 2.21855E−05 |
| LMCD1 | 1.54313711 | 2.26856E−05 |
| MAGED2 | 1.42577997 | 2.28093E−05 |
| RPGRIP1L | 2.30088761 | 2.32284E−05 |
| MT1X | 1.75550879 | 2.34342E−05 |
| REPIN1 | 1.40482269 | 2.35893E−05 |
| DNER | 2.54706 | 2.35943E−05 |
| KATNB1 | 1.41230234 | 2.40285E−05 |
| C14orf50 | 2.0041349 | 2.42509E−05 |
| IFT88 | 1.81175502 | 2.53479E−05 |
| POLQ | 1.82761614 | 2.58084E−05 |
| HSD17B13 | 2.1583746 | 2.61563E−05 |
| TSPAN8 | 1.57248017 | 2.69759E−05 |
| MAP9 | 2.17752296 | 2.70383E−05 |
| CD6 | 1.66024598 | 2.70383E−05 |
| CUEDC1 | 1.44127151 | 2.70383E−05 |
| PALMD | 1.84259482 | 2.73396E−05 |
| CCDC88C | 1.44651505 | 2.9513E−05 |
| GSTA2 | 3.04364309 | 2.99797E−05 |
| LOC728392 | 2.45352889 | 3.13987E−05 |
| SOX2 | 1.42277901 | 3.25439E−05 |
| WDR73 | 1.45128947 | 3.2565E−05 |
| KRT15 | 1.66470618 | 3.25997E−05 |
| ARVCF | 1.4675952 | 3.46454E−05 |
| UNC93B1 | 1.3350195 | 3.6432E−05 |
| FBF1 | 1.58227897 | 3.82227E−05 |
| NLRC3 | 1.6969175 | 3.93238E−05 |
| MLF1 | 2.10274167 | 3.97233E−05 |
| ACACB | 1.49814786 | 4.01764E−05 |
| ADCY9 | 1.51669291 | 4.03583E−05 |
| DIAPH2 | 1.56970385 | 4.08846E−05 |
| TCEAL3 | 1.44291146 | 4.16479E−05 |
| AGBL5 | 1.44132278 | 4.20047E−05 |
| ANKZF1 | 1.44697405 | 4.20298E−05 |
| TCEA2 | 1.52429185 | 4.23984E−05 |
| BAHCC1 | 1.49917059 | 4.27983E−05 |
| SYT17 | 1.56742434 | 4.28886E−05 |
| HSD17B8 | 1.44037694 | 4.30152E−05 |
| RPS6KA2 | 1.44445649 | 4.35723E−05 |
| PHTF1 | 1.48986592 | 4.40703E−05 |
| TTC30B | 1.71522649 | 4.43779E−05 |
| TMEM67 | 2.20416717 | 4.46512E−05 |
| PYCR1 | 1.68525202 | 4.5225E−05 |
| C11orf2 | 1.34624129 | 4.7456E−05 |
| PDE8B | 2.32876958 | 4.79301E−05 |
| GAL3ST2 | 1.52140934 | 4.82899E−05 |
| MYCL1 | 1.49285532 | 4.91023E−05 |
| TULP3 | 1.50475936 | 4.92334E−05 |
| FBLN5 | 1.48050793 | 4.97709E−05 |
| AMN | 1.65761529 | 4.99842E−05 |
| EVL | 1.38952418 | 5.22713E−05 |
| KLC4 | 1.40405768 | 5.24118E−05 |
| WNK2 | 1.41616046 | 5.30142E−05 |
| C3orf39 | 1.45324602 | 5.54577E−05 |
| LRP4 | 1.93508583 | 5.79675E−05 |
| FAM179B | 1.49020563 | 5.79675E−05 |
| DYNC2H1 | 2.39772393 | 5.80606E−05 |
| IFT81 | 1.85697674 | 6.05797E−05 |
| SYNPO | 1.43007758 | 6.05797E−05 |
| C7orf63 | 2.2475395 | 6.07346E−05 |
| LIG1 | 1.46051313 | 6.2636E−05 |
| NR2F6 | 1.37135336 | 6.26657E−05 |
| PPDPF | 1.33519823 | 6.37715E−05 |
| COQ10A | 1.57553325 | 6.42865E−05 |
| ADPRHL1 | 1.57602912 | 6.48279E−05 |
| PLXNB1 | 1.36748122 | 6.51603E−05 |
| LIPT2 | 1.57209714 | 6.54735E−05 |
| GFER | 1.38601943 | 6.57227E−05 |
| PRAF2 | 1.48691496 | 6.62534E−05 |
| MAK | 2.11010178 | 6.6389E−05 |
| LPAR3 | 1.61372461 | 6.6389E−05 |
| CEP68 | 1.43585034 | 6.86926E−05 |
| MGAT3 | 1.63032562 | 6.88196E−05 |
| SELM | 1.68910302 | 6.90845E−05 |
| PRKCDBP | 1.75929603 | 6.95654E−05 |
| GMPR | 1.74175023 | 7.09348E−05 |
| NUDT4 | 1.66108324 | 7.1223E−05 |
| TMC4 | 1.37606676 | 7.32423E−05 |
| C18orf32 | 1.4680673 | 7.49847E−05 |
| BBS4 | 1.48414852 | 7.55039E−05 |
| TTC15 | 1.37927452 | 7.55039E−05 |
| PCM1 | 1.44508492 | 7.57285E−05 |
| AHDC1 | 1.39404544 | 7.57907E−05 |
| GPT2 | 1.37898662 | 7.83202E−05 |
| KIAA0895 | 1.83866761 | 8.00835E−05 |
| UFC1 | 1.42750311 | 8.07E−05 |
| EPHX2 | 1.47972778 | 8.11114E−05 |
| AGR3 | 2.49250589 | 8.14424E−05 |
| STUB1 | 1.40578727 | 9.07013E−05 |
| MFSD2A | 1.41538916 | 9.08106E−05 |
| TM7SF2 | 1.36011903 | 9.49179E−05 |
| BCAS3 | 1.39837526 | 9.50537E−05 |
| GYLTL1B | 1.50326839 | 9.52925E−05 |
| CDT1 | 1.68706876 | 9.60694E−05 |
| EDARADD | 1.40821946 | 9.72324E−05 |
| KIAA1841 | 1.63727867 | 9.74561E−05 |
| PDLIM4 | 1.33499063 | 9.91746E−05 |
| FBXL2 | 1.70441332 | 0.000100287 |
| CCP110 | 1.62862095 | 0.000100436 |
| PLA2G6 | 1.41041592 | 0.000101028 |
| COL4A6 | 1.81881069 | 0.000101469 |
| COG7 | 1.41067778 | 0.000101469 |
| LSS | 1.46102295 | 0.00010236 |
| PITPNM1 | 1.36286761 | 0.00010236 |
| IFT74 | 1.49355699 | 0.000102847 |
| SIPA1L3 | 1.43775294 | 0.000102847 |
| WDR13 | 1.31401675 | 0.000107509 |
| ARMCX2 | 1.63758171 | 0.000108288 |
| CKB | 1.57645121 | 0.000109216 |
| STK36 | 1.48863192 | 0.000112154 |
| FN3K | 1.51834554 | 0.00011281 |
| LOC81691 | 1.62456618 | 0.000114135 |
| FAM108A1 | 1.31380714 | 0.000114728 |
| SQLE | 1.69434086 | 0.000119836 |
| KCNQ1 | 1.33310218 | 0.000122927 |
| BRF1 | 1.37864866 | 0.000124633 |
| PROS1 | 2.25991725 | 0.000125307 |
| IGSF10 | 2.12624227 | 0.000125978 |
| ZNF358 | 1.35163158 | 0.000126256 |
| CHCHD6 | 1.46348972 | 0.000133584 |
| CES3 | 1.45903662 | 0.000138413 |
| VWA2 | 1.45385588 | 0.000138791 |
| TTC5 | 1.52203224 | 0.00014006 |
| SLC27A1 | 1.39126087 | 0.000141835 |
| CYB561 | 1.37921792 | 0.000141835 |
| RPGR | 1.85326766 | 0.000142075 |
| VMAC | 1.41981554 | 0.000146443 |
| IK | 1.37718344 | 0.000148072 |
| CEP89 | 1.5127697 | 0.000148549 |
| CEBPA | 1.33935794 | 0.000149104 |
| GPX8 | 1.72869825 | 0.00015137 |
| TUT1 | 1.35214327 | 0.000152136 |
| PEX6 | 1.52324996 | 0.000155204 |
| MT1E | 1.67168253 | 0.000155534 |
| LOC441869 | 1.43946774 | 0.000157594 |
| S1PR5 | 1.51757959 | 0.0001604 |
| CD81 | 1.32468108 | 0.000161488 |
| ENPP5 | 1.75733353 | 0.000162553 |
| ZNF204P | 1.75883566 | 0.000165462 |
| C10orf81 | 1.40543082 | 0.000165462 |
| C11orf74 | 1.86106419 | 0.000171801 |
| CRTC1 | 1.42765953 | 0.000172249 |
| DDR1 | 1.36166857 | 0.000172682 |
| THSD4 | 1.53230415 | 0.000178414 |
| TAF6L | 1.35674158 | 0.000179973 |
| AKD1 | 1.62744603 | 0.000180844 |
| LZTFL1 | 1.71503476 | 0.000184545 |
| PARP10 | 1.36830665 | 0.000189223 |
| ZNF3 | 1.36744076 | 0.000189238 |
| SEMA4C | 1.40268633 | 0.000189752 |
| ZNF584 | 1.48555318 | 0.000191741 |
| NFATC1 | 1.38421478 | 0.000191741 |
| ZNF414 | 1.39531526 | 0.000194572 |
| KIAA1797 | 1.48460385 | 0.000201377 |
| C22orf23 | 1.47274344 | 0.000207275 |
| FAM113A | 1.37538478 | 0.000207701 |
| GAS6 | 1.41786846 | 0.000211066 |
| C14orf135 | 1.50529153 | 0.000227989 |
| BAIAP2 | 1.32638974 | 0.000236186 |
| TUSC1 | 1.39360539 | 0.000247174 |
| RSPH3 | 1.43059912 | 0.00024733 |
| C14orf142 | 1.62415045 | 0.000249361 |
| C13orf15 | 1.35861972 | 0.000254195 |
| PAQR7 | 1.38092355 | 0.000258484 |
| MCF2L | 1.40608658 | 0.000258709 |
| ZFPM1 | 1.60585901 | 0.000259986 |
| PARVA | 1.39640833 | 0.00026033 |
| SMPD3 | 1.41764514 | 0.000263709 |
| C7orf41 | 1.39659057 | 0.00026517 |
| TSGA10 | 1.87725514 | 0.000266725 |
| ATPIF1 | 1.34495974 | 0.000269242 |
| TRIM3 | 1.42603668 | 0.000269692 |
| CEP290 | 1.50717501 | 0.000273516 |
| SCAMP5 | 1.39934588 | 0.00027358 |
| 8-Mar | 1.39016591 | 0.000274885 |
| TSTD1 | 1.34032792 | 0.000279518 |
| ATP6V1C2 | 1.38396906 | 0.000296582 |
| BTBD3 | 1.42834347 | 0.000299561 |
| DOCK1 | 1.3556739 | 0.000307703 |
| TPRXL | 1.46505444 | 0.000308225 |
| C6orf48 | 1.36829759 | 0.000312557 |
| RRAS | 1.43157375 | 0.000312601 |
| CTU1 | 1.70766673 | 0.000313118 |
| CDON | 1.5312556 | 0.000314033 |
| LRFN3 | 1.40276367 | 0.000320189 |
| HHLA2 | 1.77249829 | 0.000325631 |
| ATP6V0A4 | 1.40856456 | 0.000331973 |
| MAZ | 1.33830748 | 0.000331973 |
| FAM131A | 1.37617082 | 0.000334759 |
| ADCK4 | 1.35866946 | 0.000345476 |
| NBPF1 | 1.42147504 | 0.000346828 |
| PLCH2 | 1.34487014 | 0.000351121 |
| TELO2 | 1.35293949 | 0.000352106 |
| ZNF469 | 1.44727917 | 0.000378978 |
| LMLN | 1.55351859 | 0.000387955 |
| NINL | 1.42267221 | 0.000388085 |
| PAIP2B | 1.46931111 | 0.000391976 |
| LRP3 | 1.34600766 | 0.000397182 |
| ZBTB45 | 1.38679613 | 0.000405 |
| AP4M1 | 1.42014443 | 0.00041951 |
| CYP2F1 | 1.38163537 | 0.000421654 |
| ARHGAP44 | 1.46862173 | 0.00042522 |
| ASMTL | 1.29539878 | 0.000447663 |
| THNSL2 | 1.45304585 | 0.000449374 |
| PWWP2B | 1.28979929 | 0.000449374 |
| ALDH1L1 | 1.33944749 | 0.000453928 |
| LRFN4 | 1.35765376 | 0.000458695 |
| ANKRD16 | 1.50341162 | 0.000468893 |
| ABCB11 | 1.85720038 | 0.000469016 |
| PSPH | 1.54491063 | 0.000469099 |
| STRA6 | 1.61958548 | 0.00046936 |
| GRTP1 | 1.3780124 | 0.00046936 |
| COL6A1 | 1.90548754 | 0.00047228 |
| LOC100506990 | 2.06901283 | 0.000472754 |
| KIAA1009 | 1.47960091 | 0.00047416 |
| SYTL1 | 1.29291891 | 0.000484701 |
| HES4 | 1.54693182 | 0.000487686 |
| NEIL1 | 1.45846006 | 0.000487686 |
| AZI1 | 1.40092743 | 0.000487686 |
| KIAA1737 | 1.39523823 | 0.000491958 |
| TTLL5 | 1.41074741 | 0.000504884 |
| SEPW1 | 1.29723354 | 0.000509229 |
| MXD4 | 1.32904467 | 0.000509323 |
| PCSK6 | 1.8750067 | 0.000512777 |
| NQO1 | 1.40130035 | 0.000519124 |
| DAK | 1.38150961 | 0.000524279 |
| SPATA7 | 1.57805661 | 0.000530373 |
| ADARB2 | 1.68685402 | 0.000530837 |
| PODXL2 | 1.36921797 | 0.000554801 |
| UGT2A2 | 1.66808039 | 0.000555928 |
| NDN | 1.45098648 | 0.000557146 |
| UBAC1 | 1.32525498 | 0.000558971 |
| ERI3 | 1.36918331 | 0.000561446 |
| MESDC1 | 1.32459189 | 0.000561446 |
| FAM13A | 1.45037916 | 0.000562906 |
| CABIN1 | 1.37646627 | 0.000581908 |
| KIAA0649 | 1.35151381 | 0.000585764 |
| SBK1 | 1.42410101 | 0.000586514 |
| NUDT14 | 1.40941995 | 0.000597249 |
| C12orf52 | 1.36403577 | 0.000605472 |
| FAM107A | 1.81948041 | 0.000607395 |
| NME2 | 1.35909489 | 0.000612032 |
| RAVER1 | 1.33417287 | 0.000638651 |
| BOC | 1.41111691 | 0.000639409 |
| MICAL3 | 1.44407861 | 0.000645699 |
| HN1L | 1.36453955 | 0.000651034 |
| PTPRT | 1.66764096 | 0.000651183 |
| ZBTB4 | 1.3320744 | 0.000652514 |
| MIB2 | 1.34379905 | 0.000656935 |
| DST | 1.42878897 | 0.000667193 |
| LRIG1 | 1.37999443 | 0.000669593 |
| ENOSF1 | 1.41462382 | 0.000670299 |
| IGSF8 | 1.33768199 | 0.000680086 |
| MXRA7 | 1.30938141 | 0.00069497 |
| THOP1 | 1.37339684 | 0.000712132 |
| ZNF688 | 1.51336829 | 0.000716478 |
| GDPD5 | 1.38067536 | 0.000716478 |
| CECR1 | 1.44192153 | 0.000724918 |
| BBS2 | 1.40792967 | 0.000760902 |
| TBC1D16 | 1.36274032 | 0.000767741 |
| PLCB4 | 1.42820241 | 0.00078212 |
| C6orf226 | 1.32994109 | 0.000790244 |
| NEK8 | 1.43237664 | 0.000797572 |
| CASZ1 | 1.32519669 | 0.000798227 |
| FAM83F | 1.30387891 | 0.000803175 |
| FAM50B | 1.45773877 | 0.000804254 |
| MED25 | 1.42685339 | 0.000826485 |
| PYCRL | 1.40030647 | 0.00084076 |
| PDXP | 1.46783132 | 0.000841656 |
| EXOSC6 | 1.34741976 | 0.000856333 |
| VSTM2L | 1.92924479 | 0.000864429 |
| SLC25A29 | 1.30866247 | 0.000882489 |
| APOD | 1.86608903 | 0.000889037 |
| LOC728743 | 1.75169318 | 0.00089053 |
| ZNF628 | 1.42007237 | 0.000892028 |
| COBL | 1.40319221 | 0.000896699 |
| TTC30A | 1.67935463 | 0.000904764 |
| RAB40C | 1.32476452 | 0.000914679 |
| WDR92 | 1.46789585 | 0.000918523 |
| BBS12 | 1.49170368 | 0.000920472 |
| SCAF1 | 1.27078484 | 0.000920472 |
| EXD3 | 1.63736942 | 0.000922835 |
| C16orf42 | 1.26458944 | 0.000924002 |
| CBX7 | 1.30724875 | 0.000931098 |
| KLHL29 | 1.52045452 | 0.000934632 |
| MTA1 | 1.28935596 | 0.000934937 |
| ZNF496 | 1.38327158 | 0.000955848 |
| ANKRD45 | 1.70738389 | 0.000963023 |
| LOC388564 | 1.93649556 | 0.000967111 |
| HAGH | 1.32213624 | 0.000998155 |
| PDGFA | 1.42863088 | 0.001019324 |
| ZFP3 | 1.42226786 | 0.001019324 |
| ST5 | 1.34063535 | 0.001032342 |
| SLC39A13 | 1.36833179 | 0.001039645 |
| XYLT2 | 1.32074435 | 0.001043171 |
| OGFOD2 | 1.37705326 | 0.001063251 |
| CCDC106 | 1.38920751 | 0.001077622 |
| C10orf57 | 1.39625227 | 0.00108256 |
| TYSND1 | 1.32704457 | 0.00108435 |
| ZNF428 | 1.25531565 | 0.001085719 |
| ZBTB7A | 1.27318182 | 0.001101095 |
| FLJ90757 | 1.41213053 | 0.001112519 |
| TMEM120B | 1.35883101 | 0.001112519 |
| KIAA1456 | 1.49996729 | 0.001115207 |
| FAM125B | 1.40872274 | 0.001117603 |
| CLSTN1 | 1.3290101 | 0.001119504 |
| SF3A2 | 1.28509238 | 0.001134443 |
| DYNC2LI1 | 1.43389873 | 0.00114729 |
| SIGIRR | 1.28806752 | 0.00114729 |
| ABHD14B | 1.32342281 | 0.001156608 |
| OSBPL5 | 1.35005294 | 0.001181561 |
| GCDH | 1.32866052 | 0.001181561 |
| GLTSCR1 | 1.31492951 | 0.001183371 |
| TMEM175 | 1.31373498 | 0.001185533 |
| TRAPPC6A | 1.3224038 | 0.001185954 |
| HSD11B2 | 1.48148593 | 0.001191262 |
| DEXI | 1.28219144 | 0.001199474 |
| TCF7 | 1.40542673 | 0.001215045 |
| B4GALT7 | 1.28277814 | 0.001225929 |
| MYBBP1A | 1.34519608 | 0.00122885 |
| ATXN7L1 | 1.41659202 | 0.001242233 |
| PIN1 | 1.30404482 | 0.001254241 |
| MT2A | 2.04000703 | 0.001255227 |
| DNAJB2 | 1.28234552 | 0.001261961 |
| EPN1 | 1.26463544 | 0.001280015 |
| TMEM61 | 1.50446719 | 0.001281574 |
| C7orf47 | 1.27854479 | 0.001321603 |
| IDUA | 1.37272518 | 0.001349843 |
| MACROD1 | 1.33230567 | 0.001350085 |
| SERPINB10 | 1.94661954 | 0.001361514 |
| ADCK3 | 1.28015615 | 0.001363257 |
| CD99L2 | 1.37191778 | 0.001364491 |
| SIVA1 | 1.26797988 | 0.001374975 |
| ST6GALNAC6 | 1.31105149 | 0.001381949 |
| KIAA0284 | 1.30334689 | 0.001396666 |
| DNASE1L1 | 1.29767606 | 0.001422038 |
| BPHL | 1.35364961 | 0.001457025 |
| KCTD17 | 1.41885194 | 0.001460503 |
| REXO1 | 1.27951422 | 0.001466253 |
| PLEFCHA4 | 1.5120144 | 0.001477764 |
| LOC202781 | 1.39766879 | 0.001490088 |
| ZCWPW1 | 1.4170765 | 0.001527816 |
| BPIFB1 | 1.57081973 | 0.001561587 |
| LRRC68 | 1.31705305 | 0.00159354 |
| PITPNM3 | 1.30084505 | 0.00159354 |
| TTC22 | 1.29235387 | 0.00159354 |
| IRF2BP1 | 1.28392082 | 0.00159354 |
| C11orf92 | 1.50310038 | 0.001602954 |
| PPP2R3B | 1.33531577 | 0.001643944 |
| GALNTL4 | 1.32355512 | 0.001671166 |
| NFIC | 1.31815493 | 0.001671166 |
| SELO | 1.29376914 | 0.001682582 |
| GPX4 | 1.30577473 | 0.001695128 |
| CYP2J2 | 1.3244996 | 0.001696726 |
| LHPP | 1.2977942 | 0.001696726 |
| DNLZ | 1.45201735 | 0.001710038 |
| DGCR6L | 1.28160338 | 0.00171044 |
| GATS | 1.34306522 | 0.001752534 |
| NAF1 | 1.46514246 | 0.001758144 |
| PAK4 | 1.32518993 | 0.001765767 |
| TMEM138 | 1.3805845 | 0.001773926 |
| D2HGDH | 1.31785815 | 0.001788379 |
| NR2F2 | 1.33842839 | 0.001803287 |
| EPB49 | 1.32650369 | 0.001819396 |
| POFUT2 | 1.31411257 | 0.001820415 |
| B3GAT3 | 1.35107174 | 0.001832824 |
| GLI4 | 1.44684606 | 0.001837393 |
| FGF11 | 1.39446213 | 0.001840765 |
| RHBDD2 | 1.26141125 | 0.001840765 |
| ZNF444 | 1.3510369 | 0.001852547 |
| PEBP1 | 1.30689705 | 0.001854974 |
| ZCCHC3 | 1.34025699 | 0.001863781 |
| LRRC37A4 | 1.4519284 | 0.001865 |
| TUBGCP6 | 1.30193887 | 0.001904076 |
| XRCC3 | 1.3864244 | 0.001922788 |
| RNF187 | 1.29592471 | 0.001936892 |
| NCRNA00265 | 1.3750193 | 0.001948591 |
| WRB | 1.40277381 | 0.001971203 |
| CHST14 | 1.38178684 | 0.001993182 |
| PIK3R2 | 1.30114605 | 0.002023385 |
| UBTD1 | 1.28646654 | 0.002023385 |
| SEC14L5 | 1.76950735 | 0.00203473 |
| SFI1 | 1.34394937 | 0.002037678 |
| DPY30 | 1.32184041 | 0.002046145 |
| HSF1 | 1.31711734 | 0.002053899 |
| NME4 | 1.30387104 | 0.002071504 |
| RBM43 | 1.40951659 | 0.002083034 |
| FAM98C | 1.274507 | 0.002089047 |
| EML2 | 1.32629448 | 0.002117113 |
| ZNF219 | 1.29662551 | 0.002118188 |
| C20orf194 | 1.37210455 | 0.002121672 |
| B4GALNT3 | 1.30834896 | 0.002163609 |
| OBSL1 | 1.305937 | 0.00217526 |
| C18orf10 | 1.32144956 | 0.002179978 |
| NAGLU | 1.27039068 | 0.002183662 |
| MUC2 | 2.27000647 | 0.002193863 |
| MGLL | 1.27904425 | 0.002205765 |
| FAM173A | 1.38467098 | 0.002209168 |
| PSIP1 | 1.34684146 | 0.002212642 |
| TSPAN1 | 1.27665824 | 0.002224043 |
| TUSC2 | 1.29490502 | 0.002232434 |
| PROM1 | 1.46799121 | 0.002239807 |
| POLD2 | 1.31983997 | 0.002243731 |
| SCRIB | 1.29183479 | 0.002243731 |
| JMJD8 | 1.24988195 | 0.002286644 |
| RBP1 | 1.29553455 | 0.002297925 |
| UTRN | 1.35691111 | 0.002362252 |
| PARP3 | 1.34735994 | 0.002369225 |
| RASSF6 | 1.39490614 | 0.002390815 |
| LOC92249 | 1.40466136 | 0.002391912 |
| OVCA2 | 1.3163436 | 0.002404409 |
| TRIM56 | 1.29535959 | 0.002427233 |
| TREX1 | 1.26637345 | 0.002431847 |
| PECR | 1.38681797 | 0.002480649 |
| FBXL14 | 1.33944092 | 0.002480649 |
| TCN2 | 1.28764878 | 0.002480649 |
| THOC3 | 1.35544993 | 0.002495975 |
| MRPL41 | 1.4462408 | 0.002497021 |
| WNT3A | 1.56505668 | 0.002502772 |
| MAP1LC3A | 1.35719631 | 0.002502772 |
| TOP1MT | 1.4172985 | 0.00251409 |
| KREMEN1 | 1.24654847 | 0.00251866 |
| LOC729013 | 1.39863494 | 0.002528217 |
| TTLL1 | 1.43077672 | 0.002625335 |
| DMPK | 1.32867357 | 0.002625335 |
| ODF2L | 1.34583296 | 0.002626872 |
| RBM20 | 1.43070108 | 0.00266198 |
| CDC42EP5 | 1.49582876 | 0.002673583 |
| ZNF608 | 1.40853604 | 0.002676791 |
| EYA1 | 1.3918948 | 0.002677512 |
| SLFN11 | 1.6901633 | 0.002694402 |
| TMEM129 | 1.29584257 | 0.002694402 |
| PEX14 | 1.32225002 | 0.002740151 |
| MAPK8IP3 | 1.26167122 | 0.002782515 |
| CDC20B | 2.92979203 | 0.002783456 |
| ROGDI | 1.30155263 | 0.00278416 |
| ABCB6 | 1.28553394 | 0.002829302 |
| NEK1 | 1.48582987 | 0.002837851 |
| TIGD5 | 1.32981321 | 0.002841309 |
| PNMA1 | 1.34478941 | 0.002879762 |
| MLXIP | 1.29784865 | 0.002879762 |
| SHANK3 | 1.49177371 | 0.002905903 |
| STEAP3 | 1.30957029 | 0.002908485 |
| CUTA | 1.27360936 | 0.002926573 |
| FOXK1 | 1.28002126 | 0.002930286 |
| MFSD7 | 1.25269625 | 0.002962728 |
| LONRF2 | 1.51428834 | 0.003024428 |
| TRIT1 | 1.41931182 | 0.003031643 |
| MFI2 | 1.33497681 | 0.003031643 |
| CYP4B1 | 1.5268612 | 0.003087739 |
| CIT | 1.29305217 | 0.003090804 |
| C8orf82 | 1.31308077 | 0.00315658 |
| PTPMT1 | 1.28651139 | 0.003168897 |
| SPHK2 | 1.30201644 | 0.003181927 |
| TTC7A | 1.28286232 | 0.003226858 |
| CLCN4 | 1.36981571 | 0.003255752 |
| MSI2 | 1.35012032 | 0.003301438 |
| ING5 | 1.41166882 | 0.003322367 |
| PFN2 | 1.3345102 | 0.003361105 |
| SGSM1 | 1.48304522 | 0.00338494 |
| DUSP28 | 1.40424776 | 0.003417564 |
| MGMT | 1.28389471 | 0.003429868 |
| TP63 | 1.59679744 | 0.003467929 |
| BTBD9 | 1.31826402 | 0.003467929 |
| IL17RC | 1.24675615 | 0.003467929 |
| ODZ4 | 1.36904786 | 0.003524126 |
| ZNF395 | 1.29186035 | 0.003586842 |
| YDJC | 1.33057894 | 0.003598986 |
| APOO | 1.34408585 | 0.003608735 |
| SVEP1 | 1.40836202 | 0.003638829 |
| RAB11FIP3 | 1.3058731 | 0.003671701 |
| TEF | 1.3271192 | 0.003677553 |
| PIGQ | 1.2693317 | 0.003740448 |
| LGALS9B | 1.36354436 | 0.003783693 |
| MAOB | 1.66197193 | 0.003808831 |
| EID2 | 1.27884537 | 0.003835751 |
| BAD | 1.25388842 | 0.003897732 |
| BTBD2 | 1.3199268 | 0.003913864 |
| WNT5B | 1.43246867 | 0.003931223 |
| SLC25A10 | 1.24603921 | 0.004010737 |
| PLK4 | 1.81340223 | 0.004056611 |
| CEP97 | 1.41538101 | 0.004071998 |
| FAM53B | 1.26253686 | 0.00411007 |
| CTSF | 1.3223521 | 0.004131025 |
| C9orf86 | 1.2153444 | 0.004156197 |
| MAST2 | 1.32022199 | 0.004165643 |
| TSKU | 1.29264907 | 0.004165643 |
| CTBP1 | 1.2796825 | 0.004188226 |
| CES2 | 1.2809789 | 0.00419032 |
| ZNF747 | 1.35584614 | 0.004211769 |
| LOC100129034 | 1.27756324 | 0.004253091 |
| HIST3H2A | 1.37492639 | 0.0043908 |
| C16orf13 | 1.2824815 | 0.00441089 |
| ITGB4 | 1.28611762 | 0.004452134 |
| MED24 | 1.28423462 | 0.004500601 |
| IYD | 1.44205522 | 0.004540332 |
| C2orf54 | 1.30578019 | 0.004584237 |
| PRRC2B | 1.28521665 | 0.004638924 |
| PHF7 | 1.38040111 | 0.004645863 |
| MFSD3 | 1.25286479 | 0.004724472 |
| PARD6G | 1.35223208 | 0.004755624 |
| POC1A | 1.58918583 | 0.00476711 |
| LAMC2 | 1.33269517 | 0.004830864 |
| RABEP2 | 1.23103314 | 0.004830864 |
| HSPB11 | 1.30028439 | 0.004881315 |
| LOC642361 | 1.32431188 | 0.004908329 |
| LIME1 | 1.30504035 | 0.0049123 |
| FLYWCH1 | 1.28311096 | 0.004926395 |
| ANG | 1.30320826 | 0.005082111 |
| QTRT1 | 1.29616636 | 0.005082111 |
| CMTM4 | 1.31610931 | 0.005122846 |
| TMEM125 | 1.26660312 | 0.005185303 |
| SLC22A18 | 1.25291574 | 0.005205062 |
| KIAA1549 | 1.32573653 | 0.005215326 |
| PRR5L | 1.28471689 | 0.0052441 |
| MOCS1 | 1.41983774 | 0.00527108 |
| LIG3 | 1.36586625 | 0.005275193 |
| CEP85 | 1.34134846 | 0.005281836 |
| NGFR | 2.00940868 | 0.005299414 |
| FBXO27 | 1.30963588 | 0.005345999 |
| B4GALT2 | 1.27095263 | 0.005369313 |
| GRINA | 1.22714784 | 0.005469662 |
| HMGN3 | 1.30614416 | 0.005501463 |
| SLC38A10 | 1.23802809 | 0.005603169 |
| PTPRF | 1.26953871 | 0.005666966 |
| GBP6 | 1.48338148 | 0.005693169 |
| BMP7 | 1.28713632 | 0.005693169 |
| SAMD1 | 1.33223945 | 0.005760574 |
| GLTPD2 | 1.38603298 | 0.005780154 |
| WDPCP | 1.43105126 | 0.005868184 |
| ZNF764 | 1.32764703 | 0.005880763 |
| SLC7A4 | 1.38094904 | 0.005896344 |
| GRB10 | 1.24234552 | 0.005898053 |
| PRICKLE3 | 1.3269405 | 0.005899727 |
| CCDC61 | 1.31458986 | 0.005914279 |
| LTK | 1.32450408 | 0.005930841 |
| ITM2C | 1.25343875 | 0.005945917 |
| TAB1 | 1.3138026 | 0.005986003 |
| WDR5B | 1.39199432 | 0.006027191 |
| EVC | 1.36532048 | 0.006041191 |
| SLC39A3 | 1.2652111 | 0.006058887 |
| NAA40 | 1.31875635 | 0.006126576 |
| ZNF696 | 1.34935807 | 0.006126723 |
| CCDC57 | 1.37984887 | 0.006169795 |
| B3GNT1 | 1.34790314 | 0.006464002 |
| SCNN1B | 1.24287546 | 0.006510517 |
| SAP30 | 1.37835625 | 0.00653315 |
| FAM3A | 1.21815206 | 0.006541067 |
| CYP27A1 | 1.39178134 | 0.006574926 |
| GMPPB | 1.26122262 | 0.006743861 |
| POLI | 1.37956907 | 0.006792284 |
| ALDH16A1 | 1.22035177 | 0.006837667 |
| MSLN | 1.33518432 | 0.006865695 |
| WDTC1 | 1.24564439 | 0.006879974 |
| RAB11B | 1.23317496 | 0.006954255 |
| HRASLS2 | 1.44393323 | 0.006995945 |
| DAGLA | 1.31649105 | 0.006995945 |
| DCXR | 1.23902542 | 0.007010789 |
| PLEKHH1 | 1.29761579 | 0.007058065 |
| NUDT16L1 | 1.24681519 | 0.007069306 |
| KLHL26 | 1.35470062 | 0.007102702 |
| NPIPL3 | 1.26640845 | 0.007118708 |
| DUOX1 | 1.28208189 | 0.007150069 |
| LTBP2 | 1.28195811 | 0.007190191 |
| TCTA | 1.30149363 | 0.007212297 |
| SPR | 1.28479279 | 0.007287193 |
| ZFYVE28 | 1.39878951 | 0.007333848 |
| AGPAT4 | 1.37723985 | 0.007347907 |
| SLC39A11 | 1.27733497 | 0.007353196 |
| TMEM150C | 1.35301424 | 0.007388326 |
| CDC42BPG | 1.26124605 | 0.007488491 |
| SLC7A1 | 1.28202511 | 0.007507941 |
| COL4A5 | 1.32559521 | 0.007512488 |
| PAX7 | 1.3155991 | 0.007535441 |
| ISOC2 | 1.23948495 | 0.007577305 |
| AGPAT3 | 1.26745455 | 0.007585223 |
| USP31 | 1.35428511 | 0.007618314 |
| PCSK5 | 1.29446783 | 0.007618314 |
| SLC16A5 | 1.25930381 | 0.007670005 |
| NOL3 | 1.2781252 | 0.00767895 |
| FBXL8 | 1.43124805 | 0.007687014 |
| SNRNP25 | 1.28739727 | 0.007722414 |
| CDCA7L | 1.34644696 | 0.007787269 |
| MOSPD3 | 1.27745533 | 0.007817906 |
| CACNB3 | 1.33319457 | 0.007881717 |
| ACBD7 | 1.5826075 | 0.007886797 |
| ADCY2 | 1.66275163 | 0.007889009 |
| CGNL1 | 1.27908311 | 0.007934511 |
| PLEKHH3 | 1.24634845 | 0.007946023 |
| CNNM2 | 1.38525605 | 0.007983142 |
| FIZ1 | 1.28867102 | 0.00798317 |
| DNHD1 | 1.38047028 | 0.008084565 |
| PHPT1 | 1.26190344 | 0.008084565 |
| TSPYL5 | 1.36008323 | 0.008097033 |
| IRX5 | 1.25420627 | 0.008212841 |
| STK11IP | 1.23490937 | 0.008220192 |
| CHPF | 1.27265262 | 0.00823526 |
| STOX2 | 1.3946561 | 0.00826187 |
| TTBK2 | 1.3997974 | 0.008275791 |
| CBX8 | 1.36626331 | 0.008275791 |
| PPP1R3F | 1.32059699 | 0.008334819 |
| JOSD2 | 1.48865236 | 0.008361772 |
| C17orf59 | 1.28230989 | 0.008361772 |
| DECR2 | 1.23796832 | 0.008455759 |
| TMEM143 | 1.37235803 | 0.008476405 |
| OPLAH | 1.25881928 | 0.008476405 |
| MYPOP | 1.29609705 | 0.008483284 |
| CEL | 1.93651713 | 0.008531505 |
| BCL2 | 1.39092608 | 0.00871498 |
| NGEF | 1.52005004 | 0.008775214 |
| USP21 | 1.31913668 | 0.008780827 |
| RAD9A | 1.25389182 | 0.008780827 |
| LGALS3BP | 1.24961354 | 0.008801136 |
| LGALS9C | 1.43680372 | 0.008865252 |
| UPF1 | 1.25440678 | 0.008873906 |
| LEMD2 | 1.20960949 | 0.008877864 |
| ZFP41 | 1.34143098 | 0.009044513 |
| SEPN1 | 1.26474089 | 0.009084 |
| PLLP | 1.31604938 | 0.00913286 |
| CUL7 | 1.27441781 | 0.009164349 |
| KRBA1 | 1.27792781 | 0.00923669 |
| FAM195B | 1.21801424 | 0.009241888 |
| ATG9B | 1.43120177 | 0.009248504 |
| ARHGEF17 | 1.30638434 | 0.009248504 |
| NUAK1 | 1.2674662 | 0.009299617 |
| ENDOV | 1.39721558 | 0.009324361 |
| SCARA3 | 1.32119045 | 0.009332766 |
| LAMB1 | 1.50281672 | 0.009344234 |
| CIDEB | 1.28399596 | 0.009344234 |
| KLHDC7A | 1.30138188 | 0.009386153 |
| WLS | 1.23889735 | 0.009435274 |
| FAM161B | 1.36982011 | 0.009478536 |
| PACS2 | 1.26997864 | 0.009508236 |
| SLC25A23 | 1.26489355 | 0.009521659 |
| FAM164A | 1.50789785 | 0.009626128 |
| C1orf110 | 1.3202239 | 0.00963096 |
| CENPB | 1.18615837 | 0.009652916 |
| ZNF704 | 1.33301508 | 0.009690515 |
| C19orf6 | 1.20316007 | 0.009730685 |
| KIAA0753 | 1.30653182 | 0.009784699 |
| CST3 | 1.21230246 | 0.009784699 |
| SLC41A3 | 1.25668605 | 0.00979418 |
| PEX10 | 1.27191387 | 0.009844346 |
| C12orf76 | 1.42258291 | 0.009870686 |
| SLC1A5 | 1.24890407 | 0.009910692 |
| RAP1GAP | 1.3443049 | 0.009932188 |
| GRAMD1C | 1.36938141 | 0.009956926 |
| NME3 | 1.33160165 | 0.010064843 |
| ABHD8 | 1.27046682 | 0.010270086 |
| ANKS1A | 1.28882538 | 0.010380221 |
| SLC25A38 | 1.29944952 | 0.010501494 |
| SERPINF2 | 1.3305424 | 0.010548835 |
| TP53I13 | 1.32153864 | 0.010567211 |
| PANX2 | 1.31303008 | 0.010589648 |
| ALKBH5 | 1.25805436 | 0.010606283 |
| CHST6 | 1.25428683 | 0.01060947 |
| WDR83 | 1.31345803 | 0.010637404 |
| SERPINB11 | 1.4704188 | 0.010638878 |
| SIX5 | 1.33395042 | 0.01072225 |
| KIAA0319 | 1.34703243 | 0.010736018 |
| ABCC10 | 1.26473091 | 0.01082689 |
| EPCAM | 1.2567134 | 0.010932803 |
| C15orf38 | 1.30075878 | 0.010969472 |
| AXIN2 | 1.29402405 | 0.011001282 |
| NISCH | 1.25096394 | 0.011018413 |
| IGF2BP2 | 1.30475867 | 0.011048991 |
| MOSC2 | 1.47927047 | 0.011053117 |
| KIAA1908 | 1.35564703 | 0.01110532 |
| SESN1 | 1.31752072 | 0.011207697 |
| C1orf86 | 1.28409107 | 0.011320516 |
| G6PC3 | 1.2125164 | 0.011409549 |
| B3GALT6 | 1.22733693 | 0.011440605 |
| KIF3A | 1.38292341 | 0.011569466 |
| FMO5 | 1.38477766 | 0.011656611 |
| FOXP2 | 1.37687706 | 0.011656611 |
| EP400 | 1.28435344 | 0.011755788 |
| CYP2S1 | 1.27545746 | 0.011755788 |
| VEGFB | 1.22471026 | 0.011755788 |
| TRIM32 | 1.29368942 | 0.011769481 |
| TSNARE1 | 1.3634355 | 0.011803378 |
| LSM4 | 1.23306793 | 0.012045042 |
| SAMHD1 | 1.35015325 | 0.01211293 |
| GALT | 1.33655074 | 0.012150017 |
| CHST12 | 1.29296088 | 0.012150017 |
| SUMF2 | 1.24339802 | 0.012170682 |
| C14orf80 | 1.29511855 | 0.012344687 |
| TFPI2 | 1.6495853 | 0.012357876 |
| NUDT7 | 1.51871011 | 0.012357876 |
| PNKP | 1.24958927 | 0.012357876 |
| PFKM | 1.29401217 | 0.012409059 |
| MDC1 | 1.29181732 | 0.012467682 |
| C17orf108 | 1.32080282 | 0.012502986 |
| MRPL4 | 1.22051577 | 0.012531908 |
| CTTNBP2 | 1.34156692 | 0.012602161 |
| NEK6 | 1.24934177 | 0.01272017 |
| APCDD1 | 1.37290114 | 0.012767663 |
| SNAPC1 | 1.31811966 | 0.012784092 |
| CUL9 | 1.24321273 | 0.012798949 |
| DCBLD2 | 1.29914309 | 0.012917806 |
| CHID1 | 1.23513008 | 0.012952152 |
| PELP1 | 1.19235772 | 0.012973503 |
| IL2RB | 1.87694069 | 0.012983156 |
| EBPL | 1.24533429 | 0.013071502 |
| TMEM110 | 1.29864886 | 0.013215192 |
| EGFR | 1.28277513 | 0.013226151 |
| ACAT1 | 1.27648584 | 0.013237073 |
| FADD | 1.22480421 | 0.013237073 |
| NCOR2 | 1.24365674 | 0.013251736 |
| DUSP23 | 1.18759129 | 0.0134367 |
| MIPOL1 | 1.35481022 | 0.013580231 |
| IFT52 | 1.32547528 | 0.013981771 |
| FGGY | 1.38422354 | 0.014047872 |
| ACTR1B | 1.24578421 | 0.014079645 |
| TRIOBP | 1.21105055 | 0.014166645 |
| MTR | 1.29454229 | 0.01416807 |
| C16orf45 | 1.33701418 | 0.014182012 |
| TECPR1 | 1.26017688 | 0.014209406 |
| ZNF362 | 1.2501977 | 0.014247609 |
| TMEM25 | 1.31255258 | 0.014250634 |
| ATP13A1 | 1.21286134 | 0.0142645 |
| ALDH4A1 | 1.29508866 | 0.014386525 |
| GHDC | 1.2679717 | 0.014585547 |
| USP13 | 1.6468891 | 0.014645502 |
| IQCB1 | 1.30311921 | 0.014724122 |
| PRMT7 | 1.26823696 | 0.014724122 |
| SORBS3 | 1.22860767 | 0.014731446 |
| RASA3 | 1.47946487 | 0.014788674 |
| WDR18 | 1.22894705 | 0.014815312 |
| UBB | 1.21302285 | 0.014959845 |
| ZNF626 | 1.36143599 | 0.014974802 |
| CCHCR1 | 1.25121215 | 0.01509939 |
| C12orf10 | 1.22594687 | 0.015249346 |
| RGS12 | 1.1884216 | 0.015281037 |
| GGA2 | 1.23527724 | 0.015332188 |
| C9orf21 | 1.34640634 | 0.015553398 |
| GAS2L1 | 1.27610616 | 0.015568411 |
| USP11 | 1.25199232 | 0.015568411 |
| LAGE3 | 1.2733059 | 0.015599785 |
| CHST10 | 1.36346099 | 0.015732751 |
| C1orf35 | 1.25664328 | 0.015735658 |
| CPSF1 | 1.20966706 | 0.015929418 |
| GJD3 | 1.22729981 | 0.016081967 |
| DLG5 | 1.23092203 | 0.01610673 |
| FAM83E | 1.21694985 | 0.016195244 |
| TRIM41 | 1.23404295 | 0.016320404 |
| TMEM213 | 1.41958146 | 0.016484036 |
| POR | 1.21138529 | 0.016499043 |
| LOC642852 | 1.46862266 | 0.016517072 |
| SDHAF1 | 1.24223826 | 0.016806901 |
| SIAH2 | 1.21834713 | 0.016864416 |
| ZNF532 | 1.28788883 | 0.017020986 |
| PHF17 | 1.25357933 | 0.017175754 |
| ZMYM3 | 1.30001737 | 0.0171865 |
| OCEL1 | 1.28256237 | 0.0171865 |
| RSG1 | 1.28718113 | 0.017273993 |
| NPTXR | 1.53025827 | 0.01727628 |
| LONP1 | 1.20031058 | 0.017332363 |
| GLT8D1 | 1.26957746 | 0.017460181 |
| ORAI2 | 1.41328301 | 0.017490601 |
| TIMM17B | 1.19661829 | 0.017535321 |
| HEXDC | 1.25292301 | 0.017542776 |
| UGT2A1 | 1.36534557 | 0.017548434 |
| URB1 | 1.25831813 | 0.017553338 |
| ARMC5 | 1.22604157 | 0.017553338 |
| TFF3 | 2.31909088 | 0.017587024 |
| ASPSCR1 | 1.20844515 | 0.017624999 |
| MRPS26 | 1.23168805 | 0.017646918 |
| TMEM134 | 1.2288306 | 0.017825679 |
| STK11 | 1.17914687 | 0.017837909 |
| XRRA1 | 1.39947437 | 0.017892419 |
| PYROXD2 | 1.34484651 | 0.018019021 |
| GNA11 | 1.25697334 | 0.018040997 |
| AGRN | 1.21988217 | 0.018182474 |
| PDE4A | 1.24320237 | 0.018184742 |
| MSH3 | 1.29294165 | 0.018305998 |
| DEGS2 | 1.28509551 | 0.018381891 |
| L3MBTL2 | 1.25584577 | 0.018599944 |
| C4orf14 | 1.26050592 | 0.018761187 |
| ProSAPiP1 | 1.22530581 | 0.018761187 |
| CTNNAL1 | 1.37868612 | 0.018768235 |
| SGCB | 1.36337998 | 0.018840796 |
| NT5DC2 | 1.22263296 | 0.018877812 |
| PHYHD1 | 1.27403407 | 0.018894874 |
| ZNF768 | 1.26202922 | 0.018933778 |
| TMEM109 | 1.23710661 | 0.019040413 |
| VWA1 | 1.19869747 | 0.019040413 |
| TM9SF1 | 1.24665895 | 0.019041146 |
| CLPP | 1.16917032 | 0.019115843 |
| ROM1 | 1.26671873 | 0.019116421 |
| ABHD6 | 1.29541914 | 0.019153377 |
| WDR81 | 1.23318896 | 0.019364381 |
| TBCB | 1.24205622 | 0.019442997 |
| IL27RA | 1.33040297 | 0.019493867 |
| LZTR1 | 1.26790326 | 0.019526164 |
| KDELC2 | 1.30411719 | 0.01972224 |
| CMBL | 1.34033189 | 0.019737295 |
| TMEM201 | 1.26474637 | 0.019843105 |
| ANKS3 | 1.22989376 | 0.019990665 |
| DENND1A | 1.22638955 | 0.020155103 |
| RGL1 | 1.24300802 | 0.020233871 |
| ARFIGEF38 | 1.32067809 | 0.020237336 |
| CD40 | 1.24570811 | 0.020269619 |
| ALKBH7 | 1.26247813 | 0.020284142 |
| SLC27A3 | 1.2354561 | 0.020421322 |
| TMEM93 | 1.31673383 | 0.020430106 |
| SIRT3 | 1.2475777 | 0.0205475 |
| SLC25A14 | 1.36204426 | 0.020560099 |
| IQCK | 1.28636095 | 0.020640164 |
| TCEANC2 | 1.28423081 | 0.020664899 |
| COL21A1 | 1.50109849 | 0.020759278 |
| RAB40B | 1.25324034 | 0.020759278 |
| TNS3 | 1.2532701 | 0.020795029 |
| COL7A1 | 1.57647835 | 0.020944269 |
| CEP120 | 1.31831944 | 0.021016979 |
| MCM2 | 1.29689526 | 0.021126757 |
| ABHD11 | 1.18994397 | 0.021329494 |
| LOC399744 | 1.31540057 | 0.021430758 |
| SLC22A23 | 1.24944619 | 0.021446138 |
| ATP6V0C | 1.17416259 | 0.021478528 |
| C17orf61 | 1.26534127 | 0.021518422 |
| MACROD2 | 1.37686707 | 0.021629967 |
| LRP5 | 1.24470319 | 0.021949014 |
| FBXL15 | 1.29192497 | 0.021972553 |
| PTPRU | 1.22543283 | 0.021972553 |
| MUC15 | 1.3122479 | 0.02203807 |
| MID1 | 1.27948316 | 0.022099398 |
| HOOK2 | 1.24529255 | 0.022099398 |
| CMAHP | 1.21368898 | 0.022099398 |
| SPRYD3 | 1.20858839 | 0.022099398 |
| CEP78 | 1.33075635 | 0.022122696 |
| FKBP11 | 1.26304562 | 0.022134566 |
| DHCR7 | 1.25305322 | 0.022252456 |
| PLOD3 | 1.25880788 | 0.022278867 |
| SLC29A2 | 1.2646493 | 0.02232075 |
| MAP3K14 | 1.21534306 | 0.022542624 |
| TUBGCP2 | 1.20510805 | 0.022542624 |
| C12orf74 | 1.26087188 | 0.022618056 |
| C9orf103 | 1.35312494 | 0.022704588 |
| ACSF2 | 1.24126062 | 0.022731424 |
| DBP | 1.21193124 | 0.022905376 |
| SCMH1 | 1.30660024 | 0.023010481 |
| DPYSL3 | 1.75851448 | 0.023022128 |
| SLC25A1 | 1.19992302 | 0.023167199 |
| H2AFX | 1.21471359 | 0.023460117 |
| ACO2 | 1.24219638 | 0.023491443 |
| SETD1A | 1.23864333 | 0.02358174 |
| HIGD2A | 1.19776928 | 0.02358174 |
| TNC | 1.50094825 | 0.023589815 |
| ZNF653 | 1.28833815 | 0.023589815 |
| SPG7 | 1.21091885 | 0.023768493 |
| PCP4L1 | 1.22918723 | 0.02383071 |
| IBA57 | 1.24180643 | 0.023836751 |
| C17orf101 | 1.25096951 | 0.023840587 |
| MICALL2 | 1.22125277 | 0.024144748 |
| SLC25A6 | 1.18752058 | 0.024216742 |
| HLF | 1.35897608 | 0.024265873 |
| LDHD | 1.2236788 | 0.024265873 |
| HIC1 | 1.32339144 | 0.02431121 |
| CDAN1 | 1.2574241 | 0.024430835 |
| BLVRB | 1.19730184 | 0.024565321 |
| FANCF | 1.30835319 | 0.024591866 |
| C21orf33 | 1.23065152 | 0.02463506 |
| EPB41L2 | 1.26976906 | 0.024700064 |
| RANBP1 | 1.23115634 | 0.024823686 |
| NUCB2 | 1.23698305 | 0.02484779 |
| NCKAP5L | 1.2397669 | 0.024923181 |
| ZBED1 | 1.21522185 | 0.024923181 |
| KBTBD6 | 1.4316415 | 0.025051133 |
| THADA | 1.27276897 | 0.025121918 |
| GLIS2 | 1.33309074 | 0.02512733 |
| ZNF787 | 1.16942772 | 0.025159688 |
| AES | 1.16914969 | 0.025347775 |
| C14orf169 | 1.25236913 | 0.025508325 |
| CAPN10 | 1.20119334 | 0.02551561 |
| CX3C11 | 2.03560065 | 0.02571443 |
| TP53BP1 | 1.30144588 | 0.025752829 |
| EEF2K | 1.22751357 | 0.026121177 |
| ZNF629 | 1.19878625 | 0.026179758 |
| PTK7 | 1.26249033 | 0.026187159 |
| CYB5R3 | 1.22279029 | 0.026187912 |
| GSDMB | 1.22615544 | 0.026402701 |
| ECHDC2 | 1.17956917 | 0.026402701 |
| GSDMD | 1.22611348 | 0.026430687 |
| RAB26 | 1.3029921 | 0.026534641 |
| LFNG | 1.27842536 | 0.02667787 |
| SREBF2 | 1.22653731 | 0.027051285 |
| DNAJC27 | 1.33234962 | 0.027090378 |
| TMEM178 | 1.32401023 | 0.027240857 |
| IVD | 1.24553409 | 0.027240857 |
| PEMT | 1.2385554 | 0.02725035 |
| HIST2H2BF | 1.25568147 | 0.027417938 |
| TNRC18 | 1.20092173 | 0.027612815 |
| PPP5C | 1.25860277 | 0.027781088 |
| AHSA2 | 1.33551621 | 0.027828419 |
| FAM171A1 | 1.2547829 | 0.027880091 |
| CYP2B6 | 1.89206892 | 0.02801745 |
| QSOX2 | 1.30285256 | 0.0282336 |
| SCD5 | 1.24820591 | 0.0282336 |
| CEP164 | 1.25975237 | 0.028265449 |
| RPL13 | 1.19710205 | 0.028278399 |
| BANF1 | 1.22270928 | 0.02848803 |
| ZNF777 | 1.22715757 | 0.028513321 |
| EPHX1 | 1.19634133 | 0.028554468 |
| TRPM4 | 1.19491647 | 0.028592325 |
| KIFAP3 | 1.32574468 | 0.028652927 |
| SULT1A1 | 1.35803402 | 0.028720872 |
| C1QBP | 1.2250998 | 0.028744187 |
| SH2B1 | 1.23275523 | 0.028748064 |
| CYP2B7P1 | 1.3709621 | 0.029004147 |
| CMIP | 1.18939283 | 0.029028829 |
| SLC2A11 | 1.34050851 | 0.029279513 |
| SMG6 | 1.2413887 | 0.029305629 |
| ARL2 | 1.23879567 | 0.029305629 |
| TTC7B | 1.41937755 | 0.029317704 |
| CTDP1 | 1.16949182 | 0.029509238 |
| LOXL1 | 1.29289943 | 0.02952562 |
| CDS1 | 1.24920822 | 0.030016095 |
| BOD1 | 1.24305642 | 0.030061948 |
| PTPRS | 1.25084066 | 0.030069163 |
| ARHGEF19 | 1.23306546 | 0.030316941 |
| PPAP2C | 1.19053642 | 0.030316941 |
| TRAF3 | 1.23277663 | 0.030350579 |
| ZNF707 | 1.23412475 | 0.030818439 |
| DIS3L | 1.25442333 | 0.031179257 |
| GGA1 | 1.19942103 | 0.031209924 |
| SNTB1 | 1.23919253 | 0.031230312 |
| KCTD13 | 1.22015811 | 0.031269564 |
| SOX21 | 1.25686272 | 0.031295938 |
| SLC9A3R1 | 1.19749434 | 0.031709604 |
| GLTPD1 | 1.19038361 | 0.031717891 |
| WTIP | 1.26447786 | 0.031869682 |
| RHOBTB2 | 1.26176919 | 0.032458791 |
| POLRMT | 1.19980497 | 0.032991066 |
| SERTAD4 | 1.28870378 | 0.033069887 |
| MPST | 1.16862519 | 0.033104411 |
| ZNRF3 | 1.34876959 | 0.033173043 |
| P4HA2 | 1.25705664 | 0.033701888 |
| MPV17L | 1.26662253 | 0.03402012 |
| ARHGEF18 | 1.20479337 | 0.03402012 |
| ZNF385A | 1.17649674 | 0.034069213 |
| DDAH1 | 1.28088496 | 0.034092835 |
| MLLT6 | 1.20261495 | 0.0341598 |
| CPNE2 | 1.21968246 | 0.034227225 |
| MRPS31 | 1.27242786 | 0.034296798 |
| DHODH | 1.2852554 | 0.034427626 |
| DIP2C | 1.25542149 | 0.03464283 |
| SUSD3 | 1.28440939 | 0.034683637 |
| PRKAR1B | 1.23530537 | 0.034768811 |
| CIRBP | 1.18770113 | 0.034785942 |
| CSNK1G2 | 1.13123724 | 0.034785942 |
| TCEAL1 | 1.28209383 | 0.035208866 |
| IPO13 | 1.24220969 | 0.035208866 |
| RCCD1 | 1.335678 | 0.035266459 |
| SLC23A2 | 1.23369819 | 0.035486274 |
| HSF2 | 1.24483768 | 0.035535946 |
| COG1 | 1.21528079 | 0.035737318 |
| ZNF607 | 1.28896111 | 0.035814809 |
| ZNF473 | 1.30191148 | 0.03587568 |
| PRPF6 | 1.1570728 | 0.035909989 |
| SLC7A8 | 1.24579493 | 0.035915271 |
| DMWD | 1.26441363 | 0.036031824 |
| C7orf55 | 1.20257164 | 0.036467386 |
| LOC152217 | 1.19366436 | 0.036569637 |
| TMEM223 | 1.22267466 | 0.036595833 |
| HDAC11 | 1.2172885 | 0.03684229 |
| AKT3 | 1.32799964 | 0.037008607 |
| LMTK3 | 1.29813131 | 0.037095716 |
| TRAPPC5 | 1.20831411 | 0.037095716 |
| ITFG2 | 1.23730793 | 0.037115391 |
| KJAA1161 | 1.22160862 | 0.037232096 |
| TFAP4 | 1.39134809 | 0.037263881 |
| MAP1S | 1.17464502 | 0.037440506 |
| CAPN9 | 1.39055066 | 0.037748465 |
| COG8 | 1.2314403 | 0.038062365 |
| UPF3A | 1.24255729 | 0.038707203 |
| XPNPEP3 | 1.29860558 | 0.038818491 |
| MFSD10 | 1.17159262 | 0.038901436 |
| CD8A | 1.58747274 | 0.03893846 |
| SLC25A22 | 1.24064395 | 0.039092773 |
| PAQR8 | 1.29464418 | 0.039244293 |
| HIRIP3 | 1.22398822 | 0.039367991 |
| TRIM8 | 1.18882424 | 0.039367991 |
| OAF | 1.23071976 | 0.039512526 |
| SNCA | 1.27821293 | 0.040095856 |
| 8-Sep | 1.18728437 | 0.040095856 |
| C3 | 1.52927726 | 0.040833841 |
| C17orf89 | 1.218819 | 0.041044444 |
| TRIM28 | 1.18909519 | 0.041103346 |
| CARD10 | 1.23773554 | 0.041297199 |
| TMEM141 | 1.19110714 | 0.041365589 |
| C11orf31 | 1.14760658 | 0.041444485 |
| THTPA | 1.2910393 | 0.041760045 |
| VKORC1 | 1.18718687 | 0.041892204 |
| SELENBP1 | 1.1721689 | 0.042289115 |
| DOFIH | 1.22434618 | 0.042312153 |
| BSCL2 | 1.3183409 | 0.042641173 |
| FAIM | 1.27952766 | 0.042673939 |
| ZNF503 | 1.19706599 | 0.042673939 |
| RNPEP | 1.2030262 | 0.042712204 |
| GPR153 | 1.21365345 | 0.042737806 |
| LOC147727 | 1.27577433 | 0.042987541 |
| TMEM218 | 1.29964029 | 0.043031867 |
| DDX51 | 1.2431896 | 0.043259718 |
| NBEA | 1.24270767 | 0.043259718 |
| KIAA0754 | 1.33628562 | 0.043584142 |
| P4HA1 | 1.27680255 | 0.043633316 |
| NUMA1 | 1.18675348 | 0.044086191 |
| TPRA1 | 1.18791628 | 0.044350632 |
| DHRS11 | 1.25981602 | 0.04459514 |
| TMEM216 | 1.23211237 | 0.04472713 |
| SEZ6L2 | 1.23005246 | 0.04472713 |
| AGTRAP | 1.21322042 | 0.04472713 |
| PTPLAD2 | 1.39497647 | 0.044903769 |
| PTPRCAP | 1.41832342 | 0.044929234 |
| C19orf29 | 1.20477082 | 0.044969597 |
| FAM83H | 1.17895261 | 0.045287191 |
| SP8 | 1.26481614 | 0.045370219 |
| PLEKHG4 | 1.24585626 | 0.045638621 |
| TMEM9 | 1.21047154 | 0.045968953 |
| ANKRD11 | 1.20248177 | 0.04613435 |
| PABPC4 | 1.19064568 | 0.046299186 |
| ALKBH6 | 1.2014857 | 0.046508916 |
| C19orf63 | 1.18088252 | 0.046519544 |
| GIGYF1 | 1.17275338 | 0.046738543 |
| ZNF574 | 1.23128612 | 0.046937115 |
| SDF4 | 1.16627093 | 0.046954331 |
| CAMK1 | 1.23284144 | 0.047106124 |
| TTLL4 | 1.20520638 | 0.047538908 |
| SULT1E1 | 1.4294267 | 0.047970508 |
| RAB13 | 1.1740176 | 0.047981821 |
| SMCR7 | 1.20475982 | 0.048036512 |
| SCARB1 | 1.2307995 | 0.048174963 |
| LCK | 1.30353093 | 0.048431845 |
| THBS3 | 1.1933001 | 0.048455354 |
| NCDN | 1.23307681 | 0.048579383 |
| CAD | 1.24055107 | 0.049142937 |
| EEF2 | 1.18180291 | 0.049567914 |
| DPH1 | 1.21637967 | 0.049735202 |
| ASB1 | 1.21869366 | 0.049969351 |
| NABA_CORE_MATRISOME | Ensemble of | 2.71E−07 |
| | genes Encoding | |
| | core Extracellular | |
| | matrix including | |
| | ECM | |
| | glycoproteins, | |
| | collagens and | |
| | proteoglycans | |
| NABA_ECM_GLYCOPROTEINS | Genes encoding | 8.91E−07 |
| | structural ECM | |
| | glycoproteins | |
| REACTOME_RECRUITMENT_OF_MITOTIC_CENTROSOME_PROTEINS_AND | Genes involved | 2.86E−06 |
| COMPLEXES | in Recruitment of | |
| | mitotic | |
| | centrosome | |
| | proteins and | |
| | complexes | |
| REACTOME_MITOTIC_G2_G2_M_PHASES | Genes involved | 3.98E−05 |
| | in Mitotic G2- | |
| | G2/M phases | |
| REACTOME_LOSS_OF_NLP_FROM_MITOTIC CENTROSOMES | Genes involved | 2.02E−04 |
| | in Loss of Nlp | |
| | from mitotic | |
| | centrosomes | |
| NABA_MATRISOME | Ensemble of | 2.10E−04 |
| | genes encoding | |
| | extracellular | |
| | matrix and | |
| | extracellular | |
| | matrix-associated | |
| | proteins | |
| REACTOME_CHONDROITIN_SULFATE_DERMATAN_SULFATE_METABOLISM | Genes involved | 9.82E−04 |
| | in Chondroitin | |
| | sulfate/dermatan | |
| | sulfate | |
| | metabolism | |
| REACTOME_METABOLISM_OF_LIPIDS_AND_LIPOPROTEINS | Genes involved | 9.82E−04 |
| | in Metabolism of | |
| | lipids and | |
| | lipoproteins | |
| KEGG_GLYCOSAMINOGLYCAN_BIOSYNTHESIS_CHONDROITIN_SULFATE | Glycosaminoglycan | 9.82E−04 |
| | biosynthesis - | |
| | chondroitin | |
| | sulfate | |
| REACTOME_GLYCOSAMINOGLYCAN_METABOLISM | Genes involved | 4.40E−03 |
| | in | |
| | Glycosaminoglycan | |
| | metabolism | |
| NABA_BASEMENT_MEMBRANES | Genes encoding | 7.36E−03 |
| | structural | |
| | components of | |
| | basement | |
| | membranes | |
| REACTOME_DEVELOPMENTAL_BIOLOGY | Genes involved | 7.76E−03 |
| | in Developmental | |
| | Biology | |
| REACTOME_AXON_GUIDANCE | Genes involved | 8.07E−03 |
| | in Axon guidance | |
| REACTOME_BIOLOGICAL_OXIDATIONS | Genes involved | 1.04E−02 |
| | in Biological | |
| | oxidations | |
| REACTOME_CELL_CYCLE | Genes involved | 1.82E−02 |
| | in Cell Cycle | |
| KEGG_STEROID_BIOSYNTHESIS | Steroid | 1.85E−02 |
| | biosynthesis | |
| WNT_SIGNALING | Genes related to | 2.11E−02 |
| | Wnt-mediated | |
| | signal | |
| | transduction | |
| KEGG_PEROXISOME | Peroxisome | 2.78E−02 |
| PID_INTEGRIN 1_PATHWAY | Betal integrin | 3.22E−02 |
| | cell surface | |
| | interactions | |
| KEGG_ARGININE_AND_PROLINE_METABOLISM | Arginine and | 3.56E−02 |
| | proline | |
| | metabolism | |
| REACTOME_SIGNALLING_BY_NGF | Genes involved | 4.13E−02 |
| | in Signalling by | |
| | NGF | |
| REACTOME_TRANSMEMBRANE_TRANSPORT_OF_SMALL_MOLECULES | Genes involved | 4.23E−02 |
| | in | |
| | Transmembrane | |
| | transport of small | |
| | molecules | |
| KEGG_FOCAL_ADHESION | Focal adhesion | 4.23E−02 |
| REACTOME_COLLAGEN_FORMATION | Genes involved | 4.67E−02 |
| | in Collagen | |
| | formation | |
| PID_ALPHA_SYNUCLEIN_PATHWAY | Alpha-synuclein | 4.67E−02 |
| | signaling | |
| NABA_CORE_MATRISOME | Ensemble of | 2.71E−07 |
| | genes Encoding | |
| | core extracellular | |
| | matrix including | |
| | ECM | |
| | glycoproteins, | |
| | collagens and | |
| | proteoglycans | |
| NABA_ECM_GLYCOPROTEINS | GenesEncoding | 8.91E−07 |
| | structural ECM | |
| | glycoproteins | |
| REACTOME_RECRUITMENT_OF_MITOTIC_CENTROSOME_PROTEINS_AND | Genes involved | 2.86E−06 |
| COMPLEXES | in Recruitment of | |
| | mitotic | |
| | centrosome | |
| | proteins and | |
| | complexes | |

TABLE 2B
Under-expressed Genes and Pathways

| | Fold | | | Fold | |
|---|---|---|---|---|---|
| | Change/ | | | Change/ | |
| Gene/Pathway | Description | FDR | Gene/Pathway | Description | FDR |
| FAM126A | 0.47044321 | 2.57E−13 | USP38 | 0.77604465 | 0.01002147 |
| ABCA12 | 0.54776675 | 1.99E−12 | LOC100131096 | 0.78878335 | 0.01014235 |
| ESR1 | 0.46793656 | 7.85E−12 | KPNA2 | 0.78234347 | 0.01021201 |
| SPIN4 | 0.54280156 | 3.77E−10 | DNTTIP2 | 0.77627102 | 0.01027009 |
| PTER | 0.59011532 | 4.29E−10 | PPM1B | 0.7741435 | 0.01027009 |
| DYNLT3 | 0.58759988 | 2.06E−09 | SLC19A2 | 0.77835972 | 0.01030816 |
| LPAR6 | 0.59655276 | 2.28E−09 | SLC43A3 | 0.74285594 | 0.01032916 |
| KYNU | 0.58810126 | 2.32E−09 | TMCC3 | 0.4048631 | 0.01039145 |
| DUSP10 | 0.52934498 | 3.08E−09 | RAD21 | 0.79068443 | 0.01042223 |
| ZDHHC21 | 0.60146742 | 5.22E−09 | SLC30A7 | 0.79087734 | 0.01047273 |
| POU2F3 | 0.51754048 | 1.01E−08 | TCEB1 | 0.76866124 | 0.01050149 |
| PRRG1 | 0.52569751 | 1.29E−08 | PGM2L1 | 0.81470242 | 0.01050282 |
| FAM40B | 0.41827178 | 1.33E−08 | ZNF207 | 0.78322085 | 0.01056721 |
| RAB27B | 0.63101586 | 1.81E−08 | ZFC3H1 | 0.76322477 | 0.01058595 |
| AGL | 0.60797081 | 1.94E−08 | MYOF | 0.8174365 | 0.01072082 |
| HS6ST2 | 0.50589265 | 4.17E−08 | NEDD4 | 0.75183609 | 0.01072082 |
| ERRFI1 | 0.59795439 | 5.59E−08 | SYNJ1 | 0.74797515 | 0.01072082 |
| MALL | 0.60107268 | 6.80E−08 | CHML | 0.75999034 | 0.01073602 |
| E2F2 | 0.54530533 | 9.00E−08 | LYSMD3 | 0.81359844 | 0.01075889 |
| ANKRD22 | 0.61522801 | 1.29E−07 | XDH | 0.7776994 | 0.01082657 |
| MIER3 | 0.6186614 | 1.68E−07 | STAG2 | 0.77433017 | 0.01089059 |
| LOC100505839 | 0.54012654 | 1.86E−07 | RGS1 | 0.428437 | 0.01099508 |
| LHFPL2 | 0.6290898 | 1.89E−07 | TINAGL1 | 0.76940891 | 0.01099801 |
| PPARG | 0.61457594 | 1.99E−07 | PEX13 | 0.79652854 | 0.0110079 |
| TMEM106B | 0.62973645 | 2.17E−07 | KRT6B | 0.47469479 | 0.0110079 |
| NRIP1 | 0.64071414 | 2.19E−07 | C7orf60 | 0.72826754 | 0.01101626 |
| TM4SF1 | 0.54686638 | 2.20E−07 | ATP7A | 0.78923096 | 0.01104899 |
| PLK2 | 0.62474305 | 3.09E−07 | UBTD2 | 0.78150066 | 0.01107608 |
| C8orf4 | 0.5985907 | 3.40E−07 | FGD4 | 0.76292428 | 0.01114875 |
| MBOAT2 | 0.65711393 | 3.64E−07 | HNRNPH3 | 0.78989996 | 0.01119847 |
| TMPRSS11A | 0.50012157 | 3.90E−07 | GNPNAT1 | 0.80178069 | 0.01120254 |
| HPSE | 0.63345701 | 4.27E−07 | SERPINB7 | 0.59831614 | 0.01120254 |
| SP6 | 0.50873861 | 4.58E−07 | TARS | 0.787516 | 0.01122418 |
| MCTP1 | 0.54747859 | 4.82E−07 | UBLCP1 | 0.7722069 | 0.01122648 |
| ECT2 | 0.65574576 | 6.32E−07 | GARS | 0.79199425 | 0.01132108 |
| CYR61 | 0.56382112 | 6.47E−07 | TMEM2 | 0.80301179 | 0.01138085 |
| CFL2 | 0.62040497 | 6.48E−07 | ZNF185 | 0.79182935 | 0.01143669 |
| SLC18A2 | 0.6252582 | 6.95E−07 | GDPD3 | 0.67570566 | 0.01143669 |
| OCLN | 0.66000035 | 6.98E−07 | C5orf43 | 0.79637974 | 0.01148042 |
| F2RL1 | 0.65645045 | 7.34E−07 | SIRT1 | 0.74221538 | 0.01148042 |
| OXSR1 | 0.6328292 | 7.42E−07 | MAB21L3 | 0.77571866 | 0.01156947 |
| DKK1 | 0.43751201 | 8.08E−07 | LYRM5 | 0.76896782 | 0.01156947 |
| LDHA | 0.6605144 | 8.88E−07 | IER3IP1 | 0.79267292 | 0.01158028 |
| FABP5 | 0.59566267 | 1.03E−06 | VEGFA | 0.75291474 | 0.0116188 |
| SLC38A2 | 0.65822916 | 1.05E−06 | TMSB4X | 0.72244795 | 0.01165661 |
| PDP1 | 0.66035671 | 1.06E−06 | TMEM41A | 0.77944137 | 0.01168994 |
| RND3 | 0.65234528 | 1.06E−06 | TNFAIP3 | 0.65538935 | 0.01172668 |
| CDKN2B | 0.60249001 | 1.08E−06 | INTS6 | 0.76205092 | 0.01172886 |
| SERPINB5 | 0.56356085 | 1.19E−06 | ADAM10 | 0.80151014 | 0.01175579 |
| GPNMB | 0.60704771 | 1.36E−06 | ARAP2 | 0.7953511 | 0.0118699 |
| HSD17B3 | 0.60203529 | 1.60E−06 | CNN3 | 0.80690311 | 0.01188901 |
| SERPINE2 | 0.34777028 | 1.62E−06 | SPTY2D1 | 0.77603059 | 0.01194061 |
| BZW1 | 0.67135675 | 1.72E−06 | PHF20L1 | 0.77584582 | 0.01195426 |
| MYEOV | 0.49219284 | 1.72E−06 | SERPINB1 | 0.61773856 | 0.01198815 |
| SGK1 | 0.68010617 | 1.95E−06 | HOMER1 | 0.75406296 | 0.01202166 |
| DNAJB9 | 0.66020909 | 2.02E−06 | PTK6 | 0.78404191 | 0.01213403 |
| CALB1 | 0.31335579 | 2.19E−06 | CAMSAP1L1 | 0.78125047 | 0.01215002 |
| MSR1 | 0.49696801 | 2.44E−06 | RNF11 | 0.78944171 | 0.01221391 |
| C12orf29 | 0.63475403 | 2.52E−06 | PPFIBP1 | 0.79937047 | 0.01235788 |
| PLA2G7 | 0.44181773 | 2.68E−06 | RP2 | 0.65113711 | 0.01246432 |
| CAPZA2 | 0.63650318 | 3.06E−06 | LTN1 | 0.81447306 | 0.01248787 |
| CD109 | 0.56416931 | 3.06E−06 | PAK1IP1 | 0.79300898 | 0.01253176 |
| RAPH1 | 0.69473071 | 3.27E−06 | ZNF189 | 0.76756049 | 0.01260727 |
| CERS3 | 0.63914564 | 3.33E−06 | BZW2 | 0.79754386 | 0.01273528 |
| ETV4 | 0.59884423 | 3.74E−06 | PKP1 | 0.71932402 | 0.01278409 |
| FOXN2 | 0.62642545 | 3.75E−06 | ATF1 | 0.80930096 | 0.01279478 |
| RPS6KA3 | 0.67623565 | 4.20E−06 | LIN7C | 0.79913296 | 0.01285667 |
| BCL10 | 0.65894446 | 4.20E−06 | S100A16 | 0.77701197 | 0.01291573 |
| SLC5A3 | 0.53006887 | 4.63E−06 | C1orf52 | 0.74541456 | 0.01291781 |
| STK38L | 0.62733421 | 4.91E−06 | MYO5A | 0.73515052 | 0.01297751 |
| SNX16 | 0.63704107 | 5.31E−06 | DEPTOR | 0.79024652 | 0.01303209 |
| STRN | 0.67981453 | 5.81E−06 | BAZ2B | 0.7897409 | 0.0130574 |
| HSPC159 | 0.6455435 | 6.64E−06 | MEI | 0.78969952 | 0.01306743 |
| SLCO1B3 | 0.49485284 | 6.90E−06 | NR4A2 | 0.70149781 | 0.01312925 |
| SACS | 0.62971335 | 7.24E−06 | ASNSD1 | 0.79830294 | 0.01315637 |
| PLIN2 | 0.62600964 | 7.25E−06 | CATSPERB | 0.70538226 | 0.01315637 |
| HSPA13 | 0.64757842 | 7.51E−06 | FRMD4B | 0.7805225 | 0.01321553 |
| DDX3X | 0.67297758 | 8.43E−06 | ZNF552 | 0.79768046 | 0.01346424 |
| SDR16C5 | 0.67434136 | 8.57E−06 | MFN1 | 0.81509879 | 0.01359256 |
| AMD1 | 0.67760181 | 8.91E−06 | USO1 | 0.80330724 | 0.01359256 |
| ITGB8 | 0.67887254 | 9.95E−06 | BPGM | 0.78515609 | 0.01359256 |
| SLC4A7 | 0.65708728 | 1.04E−05 | CXCL2 | 0.39887063 | 0.01359787 |
| PTP4A1 | 0.68607621 | 1.05E−05 | PPP1CC | 0.80893126 | 0.01365976 |
| HNMT | 0.68400423 | 1.05E−05 | PCNP | 0.79622567 | 0.01368486 |
| PGM2 | 0.6609215 | 1.09E−05 | S100A11 | 0.74267291 | 0.0136932 |
| FCHO2 | 0.68699512 | 1.19E−05 | ID2 | 0.75318731 | 0.0137174 |
| OAS1 | 0.63160242 | 1.20E−05 | IFRD1 | 0.42135251 | 0.0137174 |
| MAPK6 | 0.684135 | 1.20E−05 | SCFD1 | 0.80529038 | 0.01373021 |
| GRAMD3 | 0.68353459 | 1.26E−05 | EMP1 | 0.60588308 | 0.01373021 |
| ABCA1 | 0.54787448 | 1.28E−05 | LANCL3 | 0.68348747 | 0.01375217 |
| SYTL5 | 0.70638291 | 1.28E−05 | UBA6 | 0.79888098 | 0.01379958 |
| GULP1 | 0.65824402 | 1.32E−05 | RARS | 0.79366989 | 0.0138429 |
| PHLDA1 | 0.54172105 | 1.32E−05 | C7orf73 | 0.76317263 | 0.01389162 |
| NRIP3 | 0.60674778 | 1.35E−05 | LCOR | 0.81117554 | 0.01389191 |
| UGT1A10 | 0.60272574 | 1.45E−05 | PTPN12 | 0.60299739 | 0.01394062 |
| TMED7 | 0.70617128 | 1.57E−05 | IREB2 | 0.80814458 | 0.01401875 |
| ZFAND6 | 0.67093358 | 1.57E−05 | MACC1 | 0.80002988 | 0.01406745 |
| CSTA | 0.52443912 | 1.61E−05 | B4GALT5 | 0.79715598 | 0.0141339 |
| POF1B | 0.69756087 | 1.69E−05 | NAPEPLD | 0.80214979 | 0.01416807 |
| CLCA2 | 0.56020532 | 1.70E−05 | HECA | 0.72312723 | 0.01416807 |
| CYP2E1 | 0.46030235 | 1.83E−05 | SCEL | 0.59978505 | 0.01427161 |
| GPR115 | 0.51236684 | 1.94E−05 | CDK19 | 0.75633313 | 0.01433637 |
| STXBP5 | 0.68639477 | 1.95E−05 | SOCS5 | 0.78388345 | 0.01441385 |
| FHL2 | 0.69498993 | 2.13E−05 | DGKA | 0.78636133 | 0.01447758 |
| EFNB2 | 0.68000514 | 2.13E−05 | EIF3J | 0.80032433 | 0.01469173 |
| SPRY4 | 0.57593365 | 2.18E−05 | MAP1LC3B | 0.73616097 | 0.01472412 |
| FRMD6 | 0.67585426 | 2.19E−05 | IVL | 0.51954316 | 0.01487199 |
| SOX9 | 0.69148494 | 2.34E−05 | SLC38A9 | 0.78548034 | 0.01488644 |
| LYPLA1 | 0.68419869 | 2.40E−05 | TXNDC9 | 0.80599778 | 0.01499161 |
| SLC37A2 | 0.6397126 | 2.54E−05 | ARFIGAP29 | 0.79975551 | 0.01502574 |
| SLC6A14 | 0.63108881 | 2.66E−05 | CHMP1B | 0.78649063 | 0.01506495 |
| TCN1 | 0.63504893 | 2.67E−05 | CREB1 | 0.75968742 | 0.01506947 |
| STS | 0.71630909 | 2.67E−05 | AURKA | 0.7291468 | 0.01525634 |
| CLDN1 | 0.71508575 | 2.70E−05 | DENND1B | 0.78917281 | 0.01528104 |
| TGFB2 | 0.70221517 | 2.86E−05 | SP3 | 0.80275018 | 0.01547056 |
| PPP1CB | 0.69356726 | 2.96E−05 | ABCC9 | 0.75019099 | 0.01563394 |
| COPS2 | 0.70745288 | 3.20E−05 | LARP4 | 0.81575794 | 0.01573566 |
| FNDC3B | 0.70629744 | 3.27E−05 | PSTPIP2 | 0.74759876 | 0.01576062 |
| SLC9A2 | 0.70240663 | 3.45E−05 | UBAP1 | 0.72271205 | 0.01576062 |
| AHR | 0.72189199 | 3.48E−05 | GYG1 | 0.77805963 | 0.01581091 |
| CPM | 0.60903324 | 3.65E−05 | KIAA1199 | 0.54860664 | 0.01593278 |
| MRPS6 | 0.67128208 | 3.65E−05 | SNRPB2 | 0.80292457 | 0.01593921 |
| MAL2 | 0.71451061 | 4.09E−05 | FBXO34 | 0.80748644 | 0.01598506 |
| SLC9A4 | 0.68487854 | 4.09E−05 | NFAT5 | 0.80662528 | 0.01610673 |
| PLAU | 0.60117497 | 4.14E−05 | PURB | 0.80015013 | 0.01638623 |
| KCTD9 | 0.68717984 | 4.21E−05 | VTA1 | 0.795135 | 0.01638623 |
| CYP2C18 | 0.67036117 | 4.25E−05 | ZBTB38 | 0.80217977 | 0.01644708 |
| ARHGAP5 | 0.72532517 | 4.26E−05 | CYB5R2 | 0.77288599 | 0.01648404 |
| TDG | 0.7023444 | 4.31E−05 | EXOC5 | 0.81382561 | 0.01655428 |
| RALA | 0.68246265 | 4.39E−05 | CDR2L | 0.81728606 | 0.01659833 |
| ANKDD1A | 0.59706849 | 4.44E−05 | SWAP70 | 0.80565394 | 0.0167099 |
| CEACAM1 | 0.60936113 | 4.61E−05 | GLRX3 | 0.78569526 | 0.0167132 |
| TRPS1 | 0.68207878 | 4.80E−05 | MMP7 | 0.51970705 | 0.01674324 |
| GALNT5 | 0.70688281 | 4.90E−05 | C18orf19 | 0.80580272 | 0.0167524 |
| AGPAT9 | 0.54621966 | 5.57E−05 | IPPK | 0.76399847 | 0.01679915 |
| PLS1 | 0.73068821 | 5.63E−05 | BLOC1S2 | 0.76302982 | 0.01685077 |
| ABHD5 | 0.63310304 | 5.75E−05 | PDLIM2 | 0.73531533 | 0.01685769 |
| SLK | 0.70996449 | 5.86E−05 | OTUD6B | 0.74806056 | 0.01696167 |
| GNAI3 | 0.63637676 | 5.88E−05 | POLR2K | 0.78945634 | 0.01701766 |
| GPCPD1 | 0.60712726 | 6.03E−05 | C10orf118 | 0.81187016 | 0.01703642 |
| FAT1 | 0.71499305 | 6.16E−05 | RELL1 | 0.71318764 | 0.01707764 |
| CAPZA1 | 0.69202454 | 6.43E−05 | GLA | 0.60796251 | 0.01727628 |
| TUBB3 | 0.46563825 | 6.48E−05 | PLXDC2 | 0.53165839 | 0.01733236 |
| DSG3 | 0.44745628 | 6.87E−05 | L3MBTL3 | 0.77911939 | 0.01735666 |
| C6orf211 | 0.70372086 | 6.91E−05 | RUNX2 | 0.77801083 | 0.01735666 |
| SLMO2 | 0.70233453 | 7.10E−05 | CA2 | 0.4922131 | 0.01735666 |
| LOC100507127 | 0.44153481 | 7.20E−05 | PPP4R2 | 0.79532914 | 0.01736433 |
| MGAT4A | 0.70002166 | 7.36E−05 | LRRC8C | 0.67202997 | 0.01753532 |
| MST4 | 0.6716609 | 7.59E−05 | ARID4B | 0.77340187 | 0.01754278 |
| UCA1 | 0.38849742 | 7.77E−05 | SH3BGRL2 | 0.81075514 | 0.01755334 |
| TPM4 | 0.69490548 | 7.82E−05 | CPD | 0.79596928 | 0.01755334 |
| TBC1D23 | 0.70081911 | 8.08E−05 | DNAJB6 | 0.78602264 | 0.01755334 |
| C9orf150 | 0.65660789 | 8.16E−05 | RG9MTD1 | 0.78287275 | 0.01755334 |
| MPZL2 | 0.72416465 | 8.45E−05 | TXN | 0.77853577 | 0.01761555 |
| BCAT1 | 0.60155977 | 8.50E−05 | UGCG | 0.81279199 | 0.01783791 |
| PRRG4 | 0.69994187 | 8.66E−05 | ARNTL | 0.7595337 | 0.01792236 |
| ANKRD57 | 0.69957309 | 8.92E−05 | PRSS16 | 0.78421252 | 0.01793552 |
| DSEL | 0.66917039 | 8.92E−05 | RAP2A | 0.78860475 | 0.01801902 |
| CCNC | 0.72104813 | 9.50E−05 | VAMP7 | 0.78098348 | 0.01804468 |
| FGFBP1 | 0.55896463 | 9.83E−05 | JOSD1 | 0.66714848 | 0.01818247 |
| HEPH | 0.63099648 | 0.00010094 | TNFRSF12A | 0.7674609 | 0.01827299 |
| TIAM1 | 0.68576937 | 0.00010103 | EXOC1 | 0.80533345 | 0.018306 |
| FAR1 | 0.71009803 | 0.00010236 | ACOX1 | 0.77467238 | 0.01836883 |
| MANSC1 | 0.67745897 | 0.00010243 | IQGAP1 | 0.78700289 | 0.01837327 |
| TET2 | 0.69755723 | 0.00010428 | PFKFB2 | 0.79393361 | 0.01838189 |
| PTPN13 | 0.72165544 | 0.00010468 | ID1 | 0.7077695 | 0.01838189 |
| PLS3 | 0.70700001 | 0.0001063 | ELMOD2 | 0.8099594 | 0.01839339 |
| GRHL3 | 0.62055831 | 0.00011182 | SSR3 | 0.8027967 | 0.01861183 |
| TRIB2 | 0.70025116 | 0.00011358 | A2M | 0.7095884 | 0.01863194 |
| VGLL1 | 0.66984802 | 0.00011809 | PSMA3 | 0.80198438 | 0.01868687 |
| HOOK3 | 0.71748877 | 0.00012006 | TTC39B | 0.78773869 | 0.01868687 |
| FAM3C | 0.71723806 | 0.00012006 | SREK1IP1 | 0.78848537 | 0.01871407 |
| BAZ1A | 0.68508081 | 0.00012035 | DNAJC25 | 0.7466337 | 0.01872135 |
| CCDC88A | 0.65999086 | 0.00012598 | TPRKB | 0.74502201 | 0.01872135 |
| SPATA5 | 0.6904431 | 0.00012757 | DCP2 | 0.69555649 | 0.01872135 |
| SOCS6 | 0.71829579 | 0.00013007 | MCU | 0.80603403 | 0.01876119 |
| TOB1 | 0.72241206 | 0.00013331 | PVR | 0.7660582 | 0.01876119 |
| HIST1H2BK | 0.66691073 | 0.00013571 | ADRB2 | 0.75075306 | 0.01876119 |
| TOP1 | 0.71883193 | 0.00013658 | ATP13A3 | 0.82040209 | 0.0188408 |
| SRPK1 | 0.69969324 | 0.00014184 | ESRP1 | 0.80880005 | 0.0189173 |
| LRIF1 | 0.69079735 | 0.00014297 | TC2N | 0.81169068 | 0.01891942 |
| SPTSSA | 0.7084399 | 0.00014301 | ANXA3 | 0.80049136 | 0.01893378 |
| RALGPS2 | 0.7046366 | 0.00014634 | SPCS2 | 0.79971407 | 0.01893378 |
| CHMP2B | 0.70500108 | 0.00014894 | CKS2 | 0.82098525 | 0.01900244 |
| CXADR | 0.72706834 | 0.00015072 | SCOC | 0.81832985 | 0.01902309 |
| GSTA4 | 0.71794256 | 0.00015072 | SGTB | 0.63979487 | 0.01904115 |
| NAA50 | 0.72321863 | 0.00015246 | SYNM | 0.73918101 | 0.01915338 |
| SLC38A1 | 0.72718456 | 0.00015392 | NETO2 | 0.74186068 | 0.01921827 |
| GPRC5A | 0.67982467 | 0.00015492 | RAB1A | 0.79371888 | 0.01931145 |
| HRH1 | 0.57142076 | 0.00015553 | DUSP4 | 0.7679591 | 0.01932028 |
| SGPP1 | 0.60446113 | 0.00015983 | TICAM1 | 0.71976999 | 0.01949387 |
| DSC2 | 0.42009312 | 0.00016546 | RBMXL1 | 0.77176321 | 0.01959763 |
| REL | 0.70232402 | 0.00016796 | NIPAL1 | 0.75859871 | 0.01975244 |
| SERPINB8 | 0.71948572 | 0.00017411 | ARL15 | 0.78712448 | 0.01978067 |
| ESRG | 0.50616862 | 0.00017416 | SPECC1 | 0.79037053 | 0.01997725 |
| GMFB | 0.71115128 | 0.00017772 | RAET1G | 0.76619179 | 0.01997725 |
| CYCS | 0.73195986 | 0.00017997 | KLF5 | 0.81561175 | 0.01999447 |
| ATP1B3 | 0.72625915 | 0.00018351 | IFNAR1 | 0.76951871 | 0.02007723 |
| SCYL2 | 0.72159083 | 0.00018351 | USP3 | 0.77565612 | 0.0201071 |
| KRAS | 0.73375761 | 0.00018545 | FAM83C | 0.70142413 | 0.0201071 |
| ZNF518B | 0.6968451 | 0.00019734 | TRIM16 | 0.81115941 | 0.0201551 |
| PNPLA8 | 0.63204178 | 0.00020809 | NR3C1 | 0.78608488 | 0.02017233 |
| ASPH | 0.72334386 | 0.00021314 | CDC42SE2 | 0.78654377 | 0.02019726 |
| LAMA4 | 0.60508669 | 0.00021337 | CNIH4 | 0.76529362 | 0.02023387 |
| PDE5A | 0.62146953 | 0.00021406 | SLC40A1 | 0.75686068 | 0.02023734 |
| LY6D | 0.52174522 | 0.00021584 | METTL21D | 0.72136719 | 0.02031329 |
| SLC44A5 | 0.47103937 | 0.00023984 | B3GNT5 | 0.73325211 | 0.02032869 |
| XPO1 | 0.74477235 | 0.00024253 | FZD5 | 0.81737971 | 0.02042132 |
| SLC35F2 | 0.67225241 | 0.0002428 | NUP50 | 0.81619664 | 0.02042132 |
| SH2D1B | 0.59115181 | 0.00024453 | APC | 0.79253541 | 0.02042132 |
| MED13 | 0.71820172 | 0.00025206 | OSMR | 0.75202139 | 0.02042132 |
| STXBP3 | 0.71330561 | 0.00025406 | APOBEC3A | 0.41742626 | 0.02042132 |
| CTSL1 | 0.65567678 | 0.00025521 | SLC10A7 | 0.78781367 | 0.02043964 |
| CPEB4 | 0.70060068 | 0.00025668 | DTX3L | 0.80221646 | 0.02047647 |
| FLVCR2 | 0.5867205 | 0.00026148 | NR1D2 | 0.82110804 | 0.02059914 |
| RNF141 | 0.72848197 | 0.00026362 | ANXA2 | 0.81057352 | 0.02064016 |
| RAB5A | 0.71866507 | 0.00026829 | BNIP3L | 0.7921443 | 0.02065952 |
| STEAP4 | 0.73753612 | 0.00027352 | EEA1 | 0.82047062 | 0.02105772 |
| NPC1 | 0.71394763 | 0.00027481 | GLTP | 0.79057504 | 0.0211003 |
| ACTR3 | 0.67613118 | 0.00027918 | ACAP2 | 0.79259531 | 0.02112664 |
| SLC12A6 | 0.64629107 | 0.00028121 | MXD1 | 0.40192887 | 0.02113344 |
| TMEM167A | 0.73039401 | 0.0002839 | CALU | 0.82233944 | 0.02117432 |
| HBP1 | 0.71134346 | 0.00029684 | PPP2R1B | 0.82287537 | 0.02147113 |
| GPR37 | 0.64413044 | 0.00030167 | MANF | 0.79019152 | 0.02147113 |
| FAM135A | 0.73205965 | 0.00030188 | UBXN8 | 0.75092566 | 0.02147113 |
| C12orf36 | 0.67818686 | 0.00030805 | KRT13 | 0.5557856 | 0.02147113 |
| CD58 | 0.62882881 | 0.00031182 | CD55 | 0.7675448 | 0.02147853 |
| MALAT1 | 0.35629204 | 0.00031256 | PKP2 | 0.84172061 | 0.02150051 |
| YWHAZ | 0.7300418 | 0.0003126 | PLAT | 0.56494138 | 0.0215063 |
| HBEGF | 0.36825648 | 0.0003126 | NEAT1 | 0.72062622 | 0.02173452 |
| CLEC2B | 0.41375232 | 0.00031403 | NCOA3 | 0.81904203 | 0.02181149 |
| CYB5R4 | 0.62282326 | 0.00031499 | ZC3H12C | 0.79419138 | 0.02181149 |
| ATP10B | 0.73014866 | 0.00032141 | FAM49B | 0.51183042 | 0.02209803 |
| KCTD6 | 0.6982837 | 0.00032602 | CUL4B | 0.81000302 | 0.0220994 |
| ITGA2 | 0.73729371 | 0.00032753 | SCD | 0.81856731 | 0.02225105 |
| MGST1 | 0.74936959 | 0.00033476 | FXYD5 | 0.61611839 | 0.02227887 |
| CDRT1 | 0.6679511 | 0.00034261 | C3orf58 | 0.7929907 | 0.02231832 |
| SPRR1A | 0.45298366 | 0.00034579 | SOS2 | 0.78441202 | 0.02242783 |
| UGT8 | 0.6364024 | 0.00036052 | EPPK1 | 0.71847068 | 0.02247716 |
| BIRC3 | 0.63931884 | 0.00036805 | UBE4A | 0.81949437 | 0.02247809 |
| PAM | 0.73943259 | 0.00036851 | RLF | 0.76493297 | 0.02249613 |
| SMC4 | 0.72845839 | 0.00036886 | MAGT1 | 0.81754733 | 0.02251014 |
| ACTR2 | 0.7257177 | 0.00037179 | DCTN6 | 0.79087132 | 0.02255614 |
| RAB21 | 0.71063184 | 0.00038679 | ITCH | 0.81832417 | 0.02261806 |
| SEC24A | 0.74242518 | 0.00038918 | TXNL1 | 0.80210696 | 0.02270459 |
| ELL2 | 0.73642285 | 0.00039252 | EPHA2 | 0.80043392 | 0.02270459 |
| ARPC5 | 0.66218112 | 0.00039424 | SLC10A5 | 0.75403621 | 0.02270459 |
| PRDM1 | 0.56977817 | 0.00039519 | CLEC7A | 0.40086257 | 0.02273095 |
| GK | 0.56146426 | 0.00039726 | ALG6 | 0.79281819 | 0.02273251 |
| C14orf129 | 0.73022452 | 0.00040878 | TMX3 | 0.82502213 | 0.02283395 |
| CCDC99 | 0.72023731 | 0.00041286 | RAB8B | 0.51178041 | 0.02283395 |
| PRSS3 | 0.42409665 | 0.00042522 | ENPP4 | 0.82969342 | 0.02290538 |
| USP25 | 0.71934778 | 0.00042769 | SAMD4A | 0.80115193 | 0.02290538 |
| PKN2 | 0.71899998 | 0.00043042 | GNG12 | 0.81800792 | 0.02290834 |
| GPR87 | 0.73061781 | 0.00043214 | MITF | 0.79669058 | 0.02302213 |
| RORA | 0.70094713 | 0.00043625 | UBE2J1 | 0.80232214 | 0.02305656 |
| GGCT | 0.7344833 | 0.00044515 | KIAA1324L | 0.84134374 | 0.02309417 |
| ZNHIT6 | 0.76417154 | 0.00045036 | TGFBR1 | 0.77759794 | 0.02324532 |
| TMBIM1 | 0.72290834 | 0.00046454 | CHM | 0.82558253 | 0.02329511 |
| TFPI | 0.61640577 | 0.00048755 | TMEM41B | 0.80778275 | 0.02342002 |
| BCAP29 | 0.72684992 | 0.00049294 | JARID2 | 0.7674422 | 0.02350843 |
| RCOR1 | 0.70144121 | 0.00049756 | DYNC1LI1 | 0.79569175 | 0.02350861 |
| LEO1 | 0.72295774 | 0.00051807 | DNAJA1 | 0.80469715 | 0.0235662 |
| OTUB2 | 0.6388429 | 0.00052599 | CXCL3 | 0.57876868 | 0.0235662 |
| TMPRSS11D | 0.60003871 | 0.0005336 | AFTPH | 0.80550055 | 0.02358174 |
| CP | 0.73425817 | 0.000553 | SCGB1A1 | 0.68088861 | 0.02358174 |
| IKZF2 | 0.7513508 | 0.00055695 | BMP3 | 0.81011626 | 0.02365337 |
| ROD1 | 0.73886335 | 0.0005605 | CCRL2 | 0.6009859 | 0.02365337 |
| HPGD | 0.74086493 | 0.00056145 | SEL1L | 0.82277025 | 0.0238405 |
| NAPG | 0.73799305 | 0.00056145 | CASP7 | 0.81804453 | 0.0238405 |
| RIT1 | 0.7194234 | 0.00056717 | MED4 | 0.7939477 | 0.0238405 |
| CLCA4 | 0.63982609 | 0.00059724 | SLURP1 | 0.58553775 | 0.0238405 |
| PPP3R1 | 0.70906132 | 0.00060194 | C12orf4 | 0.82963799 | 0.02394378 |
| GABPA | 0.72611695 | 0.00060812 | DENR | 0.81434832 | 0.02394378 |
| SPCS3 | 0.75238433 | 0.00061101 | MKI67 | 0.65325272 | 0.02394378 |
| ITGAV | 0.74691451 | 0.00061101 | CD84 | 0.70733746 | 0.02421674 |
| LOC100289255 | 0.69618504 | 0.00061787 | PGM3 | 0.82981262 | 0.02433953 |
| ADAM9 | 0.75133718 | 0.00061987 | VPS4B | 0.81124865 | 0.02443084 |
| FIIF1A | 0.62106857 | 0.00061987 | SLC7A11 | 0.7055667 | 0.02443084 |
| GAN | 0.67925484 | 0.00062053 | CD44 | 0.77927941 | 0.02445288 |
| EIF1AX | 0.76260769 | 0.00062186 | SLC1A1 | 0.75927386 | 0.02456729 |
| WASL | 0.74896466 | 0.00062186 | CLPX | 0.80928724 | 0.024572 |
| UBE2W | 0.64239921 | 0.00063811 | MOSPD1 | 0.80026606 | 0.02459523 |
| RCAN1 | 0.71096698 | 0.00064856 | ZC3H15 | 0.80450651 | 0.02467764 |
| SSR1 | 0.7514502 | 0.00065077 | RABIIA | 0.80437379 | 0.02482369 |
| PHACTR2 | 0.75203507 | 0.00065103 | DNAJB1 | 0.80659609 | 0.02483132 |
| NCK1 | 0.73821734 | 0.00065616 | SC5DL | 0.81585449 | 0.02492318 |
| SDS | 0.43860257 | 0.00065851 | PON2 | 0.79911935 | 0.02492318 |
| ZNF460 | 0.6508334 | 0.00066048 | WAC | 0.80996863 | 0.02494557 |
| SPAG9 | 0.7041979 | 0.00066393 | IRAK2 | 0.78621119 | 0.02498706 |
| ETFA | 0.7376278 | 0.0006674 | MAN2A1 | 0.80945847 | 0.02501316 |
| TBL1XR1 | 0.77064376 | 0.00066959 | NRP1 | 0.75842343 | 0.02501316 |
| MET | 0.75295132 | 0.00066959 | NFKBIA | 0.64409994 | 0.02509502 |
| LOC100499177 | 0.6435527 | 0.00066959 | ZNF143 | 0.78375832 | 0.02519086 |
| RC3H1 | 0.71187912 | 0.00067619 | OSTC | 0.81380824 | 0.02520621 |
| PPP1R15B | 0.72604754 | 0.000685 | DHX15 | 0.80218767 | 0.0252546 |
| RBMS1 | 0.72833819 | 0.00069497 | USP32 | 0.69625972 | 0.02547673 |
| PAPSS2 | 0.73311321 | 0.00070388 | CMAS | 0.80689954 | 0.02563124 |
| FGFR1OP2 | 0.72583355 | 0.00070539 | ATP6V1G1 | 0.79750807 | 0.02563124 |
| PHF6 | 0.74176092 | 0.00071648 | ARPC3 | 0.74025507 | 0.02567149 |
| RAB27A | 0.69715587 | 0.00072005 | PTAR1 | 0.82246466 | 0.02577645 |
| MAP4K4 | 0.69994514 | 0.00072785 | ABCE1 | 0.8206001 | 0.02577645 |
| PRKAR2B | 0.7353908 | 0.00074015 | ZNF260 | 0.81726679 | 0.02577645 |
| ANXA1 | 0.73823795 | 0.00074408 | VNN1 | 0.47957675 | 0.02591115 |
| LOC100134229 | 0.73183087 | 0.00074435 | TPM3 | 0.77578302 | 0.02596422 |
| OSTM1 | 0.71670885 | 0.00075171 | CNNM1 | 0.75796579 | 0.02596422 |
| SMOX | 0.59247896 | 0.00075968 | MED21 | 0.78624253 | 0.02601824 |
| RTKN2 | 0.67259731 | 0.00076669 | GM2A | 0.80553342 | 0.02604295 |
| TMEM64 | 0.751443 | 0.00076931 | PSMC2 | 0.81330981 | 0.02617976 |
| BRWD3 | 0.70874449 | 0.00077331 | RAP1B | 0.79847594 | 0.02618716 |
| YTHDF3 | 0.73166588 | 0.00077638 | CYP4X1 | 0.71483031 | 0.02618716 |
| CLDN4 | 0.71007023 | 0.00077802 | PHTF2 | 0.81641271 | 0.0262022 |
| MMP1 | 0.55376446 | 0.00077869 | UBE2V2 | 0.81033911 | 0.02626899 |
| KCNN4 | 0.68465172 | 0.00079015 | ARHGAP20 | 0.78890875 | 0.02632695 |
| CLDN12 | 0.76454862 | 0.0007909 | RHBDL2 | 0.79592484 | 0.0264027 |
| COQ10B | 0.71874588 | 0.00079995 | SMAP1 | 0.81113172 | 0.02649101 |
| LRP12 | 0.71964731 | 0.00080097 | KRT10 | 0.68898712 | 0.02653464 |
| FOSL1 | 0.51166802 | 0.00082386 | RFK | 0.80461614 | 0.02655103 |
| PARD6B | 0.74223837 | 0.00082622 | RAP1GDS1 | 0.8420239 | 0.02657993 |
| LOC439990 | 0.69267458 | 0.00083354 | MAPK1IP1L | 0.82200085 | 0.02658191 |
| PDLIM5 | 0.76185114 | 0.00084129 | SLC35A5 | 0.81757126 | 0.02659754 |
| LTBP1 | 0.73928714 | 0.00084166 | GDAP2 | 0.776095 | 0.02667787 |
| HIGD1A | 0.74108416 | 0.00084269 | MIB1 | 0.82312043 | 0.02681784 |
| RANBP6 | 0.72113191 | 0.00085429 | ITPR2 | 0.72381288 | 0.02688482 |
| AFF4 | 0.75419694 | 0.00086212 | PGRMC2 | 0.82715791 | 0.02695215 |
| RCBTB2 | 0.72276464 | 0.00088071 | RAB14 | 0.8177047 | 0.02700102 |
| DEFB1 | 0.56084482 | 0.00088306 | ARL4A | 0.82412052 | 0.02702553 |
| SORBS1 | 0.69135874 | 0.00090133 | RYBP | 0.69095215 | 0.02702816 |
| LACTB2 | 0.75713601 | 0.00092553 | TDP2 | 0.68722637 | 0.02707132 |
| DAB2 | 0.69448887 | 0.00092633 | CBX3 | 0.80911237 | 0.02714575 |
| ZNF431 | 0.70801523 | 0.00092668 | TBC1D15 | 0.79826732 | 0.02725035 |
| MAN1A1 | 0.74578309 | 0.00093774 | ZNF292 | 0.79336479 | 0.02727831 |
| RNF19A | 0.7499563 | 0.00094857 | DEK | 0.79668216 | 0.02738693 |
| SRD5A3 | 0.68412211 | 0.00094857 | GTF2F2 | 0.79408033 | 0.0273958 |
| SDCBP2 | 0.69112547 | 0.00096472 | CCNG2 | 0.66348611 | 0.02746122 |
| GLS | 0.55743607 | 0.00096829 | FBXW7 | 0.77030162 | 0.02750752 |
| ARRDC3 | 0.73257404 | 0.00098514 | NCOA7 | 0.67006969 | 0.02759494 |
| PDZD8 | 0.74504511 | 0.00101932 | SLC39A10 | 0.81569938 | 0.02762611 |
| NT5C2 | 0.74411832 | 0.00102102 | CXCL1 | 0.5037887 | 0.02773044 |
| DDX52 | 0.74116607 | 0.00102436 | LMBRD2 | 0.79862543 | 0.02773263 |
| ZNF326 | 0.73410121 | 0.00104743 | RNF139 | 0.77894417 | 0.0277779 |
| SDCBP | 0.51524162 | 0.00106089 | ATXN3 | 0.81712764 | 0.02778695 |
| TAB2 | 0.73583939 | 0.00106325 | HMGCS1 | 0.83634026 | 0.02780334 |
| MDFIC | 0.75928971 | 0.00107939 | GAB1 | 0.75314903 | 0.02799812 |
| FAM126B | 0.65824303 | 0.00109786 | DR1 | 0.79711312 | 0.02810783 |
| MAT2A | 0.76256991 | 0.00110997 | TJP1 | 0.815017 | 0.02814271 |
| SAMD9 | 0.60678126 | 0.00110997 | SSFA2 | 0.81751861 | 0.02821836 |
| OSBPL8 | 0.69459764 | 0.00111029 | SH3GLB1 | 0.80551167 | 0.02824311 |
| LIG4 | 0.73079298 | 0.0011228 | EDIL3 | 0.73606278 | 0.02837228 |
| THRB | 0.76151823 | 0.00114313 | CMTM6 | 0.73956197 | 0.02838961 |
| TNFRSF10D | 0.62060304 | 0.00114435 | PIK3C2A | 0.83154276 | 0.02851279 |
| RIOK3 | 0.73962901 | 0.00115102 | PHACTR4 | 0.82152956 | 0.02867344 |
| 6-Mar | 0.69528665 | 0.00117913 | CD86 | 0.44546002 | 0.02875144 |
| VPS26A | 0.74010152 | 0.0012058 | RSL24D1 | 0.80075639 | 0.02876288 |
| GRHL1 | 0.74125467 | 0.00121284 | MAP4K3 | 0.82252973 | 0.02880875 |
| SEC23A | 0.74746817 | 0.00122351 | C4orf32 | 0.73140848 | 0.02889681 |
| CLOCK | 0.75080448 | 0.00124549 | TGIF1 | 0.80327776 | 0.02900415 |
| SAT1 | 0.70085873 | 0.00128002 | NFYA | 0.79091615 | 0.02900415 |
| POLB | 0.7265576 | 0.00129411 | XRCC4 | 0.79014548 | 0.02906143 |
| TAF13 | 0.74566967 | 0.00129461 | BACH1 | 0.60345946 | 0.02933929 |
| DSC3 | 0.67776861 | 0.00129939 | PRPF18 | 0.79195926 | 0.02934951 |
| SAMD8 | 0.73394378 | 0.00131822 | HSPA5 | 0.82254051 | 0.02939332 |
| NPEPPS | 0.7437029 | 0.00132561 | COBLL1 | 0.80869858 | 0.02939332 |
| TPD52 | 0.75898328 | 0.00135933 | STRN3 | 0.81460651 | 0.02940888 |
| NCEH1 | 0.7474324 | 0.00136541 | C16orf52 | 0.80347457 | 0.02940888 |
| AP1S3 | 0.80504206 | 0.00136961 | ACADSB | 0.81872232 | 0.02951968 |
| USP53 | 0.75319991 | 0.00137958 | CLCF1 | 0.79372787 | 0.02959393 |
| EDEM1 | 0.75561796 | 0.00139667 | SBDS | 0.82630688 | 0.02972834 |
| MBNL1 | 0.74932328 | 0.00141178 | C1orf96 | 0.73892616 | 0.02980835 |
| TMEM33 | 0.74560237 | 0.00141178 | SVIL | 0.77354524 | 0.02993904 |
| NMU | 0.50565668 | 0.00141984 | FRS2 | 0.82504155 | 0.02998364 |
| CCPG1 | 0.74604118 | 0.0014299 | DNAJB14 | 0.79384122 | 0.02998364 |
| TBK1 | 0.73752066 | 0.00144402 | IL8 | 0.12605808 | 0.02998364 |
| PCMTD1 | 0.75791312 | 0.00146293 | GJB4 | 0.79743165 | 0.03001609 |
| SMNDC1 | 0.72111534 | 0.00147433 | UBE2E1 | 0.8132693 | 0.03004003 |
| ARNTL2 | 0.73486575 | 0.00151723 | PRC1 | 0.76311242 | 0.03009422 |
| CHPT1 | 0.72326837 | 0.00151723 | KPNA4 | 0.79641384 | 0.03021352 |
| SEC61G | 0.7105942 | 0.00151723 | ALDH3B2 | 0.80496463 | 0.03021519 |
| SHISA2 | 0.59853622 | 0.00152782 | ARFIP1 | 0.81639333 | 0.03031551 |
| XIST | 0.44631578 | 0.00155743 | BMPR2 | 0.83541357 | 0.03031694 |
| TMOD3 | 0.77533314 | 0.00157527 | PUS10 | 0.73256187 | 0.03037422 |
| HERC4 | 0.73058905 | 0.00159354 | CENPN | 0.76828791 | 0.03047261 |
| FEM1C | 0.76590656 | 0.00160833 | YES1 | 0.82057502 | 0.03053073 |
| TFRC | 0.7570632 | 0.0016402 | ZNF468 | 0.84177205 | 0.03072911 |
| F8A1 | 0.7386134 | 0.00164374 | PIK3CG | 0.53271288 | 0.03078134 |
| ATP1B1 | 0.76704609 | 0.0016534 | LPCAT2 | 0.61892931 | 0.03081115 |
| ZDHHC13 | 0.75504945 | 0.00166529 | MAGOHB | 0.77202271 | 0.03087813 |
| ERV3.1 | 0.68654538 | 0.00167391 | PGGT1B | 0.81716901 | 0.03087848 |
| TMEM30A | 0.75615819 | 0.00169183 | SIKE1 | 0.81047669 | 0.03087848 |
| CCNYL1 | 0.74297343 | 0.00169817 | C15orf52 | 0.7677753 | 0.03095296 |
| IBTK | 0.76516915 | 0.0017406 | CHST4 | 0.75379626 | 0.03109953 |
| KLF6 | 0.64386779 | 0.0017406 | SLC28A3 | 0.80134905 | 0.03115551 |
| MAP2K4 | 0.73093628 | 0.00175469 | GTDC1 | 0.77009529 | 0.03131057 |
| PICALM | 0.60342183 | 0.00178068 | ITPRIP | 0.62964124 | 0.03136065 |
| DCUN1D1 | 0.78777005 | 0.00178761 | PERP | 0.81957926 | 0.03145735 |
| SRP19 | 0.73007773 | 0.00179995 | PSMD5 | 0.81822219 | 0.03147226 |
| GNE | 0.76363264 | 0.00180792 | CNIH | 0.8396771 | 0.03158417 |
| TMEM56 | 0.72176614 | 0.00184076 | PDE4B | 0.15925174 | 0.03166939 |
| NUS1 | 0.76925969 | 0.00185255 | FAM105A | 0.76759455 | 0.03184924 |
| TMED5 | 0.75920484 | 0.00185255 | GABRE | 0.72174883 | 0.03184924 |
| PMAIP1 | 0.61359208 | 0.00185497 | UHMK1 | 0.83795019 | 0.03186968 |
| TM9SF3 | 0.76920471 | 0.00186378 | CDK6 | 0.84259905 | 0.03206511 |
| ARL8B | 0.75277703 | 0.001865 | GSPT1 | 0.81333116 | 0.03211789 |
| CSTB | 0.7246213 | 0.0018664 | CLINT1 | 0.84129485 | 0.03258105 |
| TAOK1 | 0.76340931 | 0.00187476 | SPTLC1 | 0.82243139 | 0.03262099 |
| FRK | 0.74737271 | 0.00187862 | OXR1 | 0.82634351 | 0.03273304 |
| KRT6A | 0.50297318 | 0.00188266 | SYNCRIP | 0.82737388 | 0.03294625 |
| ZRANB2 | 0.73683865 | 0.00188671 | TWSG1 | 0.82516604 | 0.03294625 |
| MAOA | 0.75804286 | 0.00190091 | TUFT1 | 0.78129892 | 0.03294625 |
| UBE2K | 0.75499291 | 0.00193919 | FAM98A | 0.82227343 | 0.03311064 |
| ZCCHC6 | 0.64117131 | 0.00197834 | ANGPTL4 | 0.62447345 | 0.03316298 |
| TACC1 | 0.73591479 | 0.00201604 | SPIN1 | 0.82919111 | 0.03336936 |
| TRAM1 | 0.76688878 | 0.00202235 | FTSJD1 | 0.82751547 | 0.03348945 |
| PNRC2 | 0.76237127 | 0.00202235 | THBS1 | 0.3372848 | 0.03405027 |
| CDC25B | 0.73376831 | 0.00205757 | YPEL2 | 0.83006226 | 0.03422723 |
| MTHFD2 | 0.71278467 | 0.0020715 | C1GALT1C1 | 0.82711113 | 0.03422723 |
| ARL5B | 0.65205708 | 0.00208123 | SFT2D2 | 0.79342076 | 0.03422723 |
| VBP1 | 0.7564177 | 0.00208303 | NBPF14 | 0.62423931 | 0.03436711 |
| IRS1 | 0.74430144 | 0.00209694 | APPBP2 | 0.81820437 | 0.03439503 |
| GALNT1 | 0.75884893 | 0.0021133 | SUB1 | 0.79595423 | 0.03442763 |
| CD68 | 0.69932459 | 0.0021133 | CSTF2 | 0.81280844 | 0.03457978 |
| ALDH1A1 | 0.78129241 | 0.00211381 | SERPINB13 | 0.74386568 | 0.03462984 |
| GALNT3 | 0.7706992 | 0.00216886 | TAF12 | 0.75776079 | 0.03465156 |
| ANKRD50 | 0.77616647 | 0.00217264 | EAF2 | 0.73385631 | 0.03465156 |
| PMP22 | 0.44713619 | 0.00220309 | ACER2 | 0.81769965 | 0.03468364 |
| ARF4 | 0.76387404 | 0.00223255 | KIAA1370 | 0.8310723 | 0.03478594 |
| ERO1L | 0.75005002 | 0.00224373 | C6orf115 | 0.7920281 | 0.03480856 |
| KIAA1033 | 0.74890236 | 0.00224373 | TMEM161B | 0.82837568 | 0.03482004 |
| UBASH3B | 0.73513497 | 0.00225969 | SERPINB4 | 0.58217203 | 0.03526646 |
| CARD6 | 0.74899398 | 0.00228664 | TMEM206 | 0.76722577 | 0.03530246 |
| RABGEF1 | 0.71844668 | 0.00230748 | TMEM87A | 0.81927656 | 0.03544177 |
| MZT1 | 0.71720898 | 0.00230944 | TAOK3 | 0.79902307 | 0.03567122 |
| ASPHD2 | 0.74295902 | 0.00238373 | KIF5B | 0.83603725 | 0.03581481 |
| 2-Mar | 0.72623707 | 0.00241931 | ATP6AP2 | 0.81457493 | 0.03586138 |
| PPP1R12A | 0.72959311 | 0.00243185 | SPRR3 | 0.55146539 | 0.03606441 |
| TRA2A | 0.7429305 | 0.00243585 | BTBD10 | 0.80108306 | 0.03618119 |
| TRAPPC6B | 0.73528091 | 0.00244989 | CBR4 | 0.81257455 | 0.03620449 |
| RAP2C | 0.68175561 | 0.0024659 | LAD1 | 0.80458232 | 0.03629508 |
| C6orf62 | 0.75844544 | 0.00251409 | SMC2 | 0.82005575 | 0.03648829 |
| PPIP5K2 | 0.78387164 | 0.00252188 | MOSPD2 | 0.61436673 | 0.03648829 |
| TGFBI | 0.52785345 | 0.00252749 | NPAS2 | 0.83232392 | 0.03656964 |
| RB1 | 0.77191438 | 0.00252877 | FBXO32 | 0.80298304 | 0.03658334 |
| IMPA1 | 0.78178293 | 0.00254095 | PLEKHA2 | 0.80322887 | 0.03677678 |
| TNPO1 | 0.78650015 | 0.00256633 | KLHL2 | 0.79563549 | 0.03677678 |
| FBXO28 | 0.77608259 | 0.00259197 | RPH3AL | 0.79452691 | 0.03677678 |
| GALNT7 | 0.78732986 | 0.0026183 | AGFG1 | 0.79019227 | 0.03677678 |
| C1D | 0.71982264 | 0.00262033 | MYO6 | 0.83241148 | 0.03684746 |
| ACVR2A | 0.74257908 | 0.00262047 | AEBP2 | 0.80355723 | 0.03686652 |
| FAM18B1 | 0.76176472 | 0.00262281 | CREB3L2 | 0.84749284 | 0.03709572 |
| CXCL6 | 0.33096087 | 0.00262687 | RANBP9 | 0.81802251 | 0.03709572 |
| ERBB2IP | 0.7639335 | 0.00266838 | KLHL15 | 0.65857368 | 0.03709572 |
| APOBEC3B | 0.59242482 | 0.00270511 | CUL3 | 0.8096363 | 0.03710186 |
| DHRS9 | 0.75871115 | 0.002728 | RAB22A | 0.80433101 | 0.03711539 |
| PIGA | 0.73677237 | 0.00273775 | OSBPL11 | 0.78407533 | 0.0371207 |
| DUSP5 | 0.6422383 | 0.00276958 | KIAA1539 | 0.69819167 | 0.03714167 |
| CLIC4 | 0.73379796 | 0.00278346 | DLG1 | 0.83009251 | 0.03726826 |
| TMEM139 | 0.75516298 | 0.00278911 | UBXN2B | 0.7072684 | 0.03738914 |
| SMAGP | 0.75555643 | 0.00280753 | IRAK4 | 0.79536496 | 0.03758668 |
| PDCD4 | 0.75886671 | 0.00281775 | PI3 | 0.58243222 | 0.03758668 |
| PSMC6 | 0.75273204 | 0.00282496 | C2orf69 | 0.80329365 | 0.03766295 |
| MMP13 | 0.57119817 | 0.00284506 | ZFAND2A | 0.77084332 | 0.03768355 |
| LLPH | 0.73355098 | 0.00288026 | APAF1 | 0.66297493 | 0.0378646 |
| WBP5 | 0.71785926 | 0.0028814 | GCOM1 | 0.68735303 | 0.03797817 |
| ANKRD36 | 0.67810421 | 0.0028814 | CA13 | 0.80329168 | 0.03802656 |
| ERGIC2 | 0.76423191 | 0.00290561 | CASP3 | 0.82104836 | 0.03806237 |
| KLF3 | 0.78570378 | 0.00290614 | CPEB2 | 0.77921871 | 0.03806237 |
| ZNF770 | 0.78511401 | 0.00290848 | IPCEF1 | 0.7139869 | 0.03808773 |
| ATP11B | 0.75855302 | 0.00291572 | CHIC1 | 0.82883135 | 0.0381983 |
| SLC16A7 | 0.7565461 | 0.00298357 | TMTC1 | 0.78485797 | 0.03831128 |
| ST3GAL4 | 0.72572041 | 0.00300271 | USMG5 | 0.79549212 | 0.03832104 |
| PPP3CA | 0.7448162 | 0.00304887 | FRYL | 0.84203988 | 0.03853779 |
| ZNF117 | 0.50142805 | 0.00306525 | RASAL1 | 0.75179941 | 0.0387072 |
| KDM6A | 0.77213154 | 0.00308418 | NBN | 0.83154425 | 0.03872393 |
| PLXND1 | 0.72142004 | 0.00308418 | HIVEP2 | 0.78765473 | 0.03881849 |
| MIER1 | 0.73557856 | 0.00313244 | TXLNG | 0.83712784 | 0.03882687 |
| OVOL1 | 0.62502792 | 0.00317568 | DOCK5 | 0.64601096 | 0.03890144 |
| SERINC1 | 0.75179781 | 0.00321045 | LPHN2 | 0.79892749 | 0.03891655 |
| RNF13 | 0.72052005 | 0.00322686 | CRNKL1 | 0.798853 | 0.03894719 |
| ZNF323 | 0.77734232 | 0.00324034 | LYPLAL1 | 0.79886604 | 0.03899625 |
| NCOA4 | 0.74867373 | 0.00324034 | SPPL2A | 0.80742034 | 0.03902383 |
| MTAP | 0.75495838 | 0.00324226 | CORO1C | 0.7980739 | 0.03903911 |
| NUFIP2 | 0.77357636 | 0.00325406 | PANK3 | 0.83224164 | 0.03915089 |
| EREG | 0.33784392 | 0.00333776 | RMND5A | 0.79488445 | 0.03951253 |
| RAB9A | 0.75777512 | 0.00340898 | SKIL | 0.76881016 | 0.03955317 |
| CTSL2 | 0.55240955 | 0.00342468 | EXOC6 | 0.81125111 | 0.03955891 |
| TMEM87B | 0.78519368 | 0.00346666 | LOC100294145 | 0.80974179 | 0.03965787 |
| NCKAP1 | 0.78570783 | 0.00352262 | CYLD | 0.79867583 | 0.03971547 |
| ACTG1 | 0.76392092 | 0.00353277 | C6orf204 | 0.77428898 | 0.03971547 |
| STEAP1 | 0.70400557 | 0.0035547 | MAP3K5 | 0.80607409 | 0.03976224 |
| C20orf54 | 0.6725607 | 0.00357863 | PRKAA2 | 0.82840521 | 0.03988755 |
| GTF2A2 | 0.75863446 | 0.00358684 | CHUK | 0.81785294 | 0.04058768 |
| LAMP2 | 0.72705142 | 0.0035881 | SNX6 | 0.81732751 | 0.04097796 |
| B4GALT4 | 0.76856871 | 0.00359353 | PSMB2 | 0.82520067 | 0.04109294 |
| ETFDH | 0.75965073 | 0.00359783 | F3 | 0.84871606 | 0.04152053 |
| BLNK | 0.75809879 | 0.00362427 | CHST2 | 0.77943848 | 0.04178592 |
| FREM2 | 0.72246394 | 0.00366469 | STX3 | 0.67806804 | 0.04184764 |
| PSMD12 | 0.76433814 | 0.00368788 | MBD2 | 0.8052338 | 0.04189529 |
| SRP72 | 0.7794528 | 0.00375595 | MKLN1 | 0.82564266 | 0.04192489 |
| PLEKHF2 | 0.77591424 | 0.0038141 | LNPEP | 0.81160431 | 0.04207684 |
| TMX1 | 0.77242467 | 0.00382017 | USP15 | 0.57814041 | 0.042141 |
| CD2AP | 0.78829185 | 0.00383168 | QKI | 0.66036133 | 0.04236353 |
| SPIRE1 | 0.74145864 | 0.0038936 | DERL2 | 0.80411723 | 0.0425095 |
| MYD88 | 0.71278412 | 0.00392321 | ZMAT3 | 0.81595879 | 0.04264891 |
| SLMAP | 0.80047015 | 0.00393122 | ARFGEF1 | 0.8346722 | 0.04298754 |
| TUBB6 | 0.64642059 | 0.00397194 | ERP44 | 0.80464897 | 0.04298754 |
| ADAMDEC1 | 0.56927435 | 0.00403827 | HR | 0.7668347 | 0.04298754 |
| BCL2L15 | 0.7904988 | 0.00404876 | PITPNC1 | 0.77723239 | 0.04308056 |
| DDX21 | 0.77375237 | 0.0040688 | CCDC59 | 0.76646023 | 0.04319013 |
| TOPORS | 0.72470814 | 0.00408953 | PHF14 | 0.83670922 | 0.0432236 |
| ARMC1 | 0.78022166 | 0.0041395 | ACP5 | 0.70586156 | 0.04325972 |
| DTWD2 | 0.7787722 | 0.0041562 | ARPC2 | 0.79251427 | 0.04329313 |
| FMR1 | 0.77028713 | 0.00419389 | WDFY3 | 0.81539874 | 0.04355816 |
| LIN54 | 0.74726623 | 0.00423614 | STK17B | 0.59142405 | 0.04356623 |
| KRT23 | 0.7309985 | 0.00423614 | ATL3 | 0.81419607 | 0.04369002 |
| CAV2 | 0.77823069 | 0.00428967 | FAM84B | 0.81682318 | 0.04373954 |
| KLHL24 | 0.78910432 | 0.00432043 | SRSF1 | 0.84262736 | 0.04402008 |
| EPB41L5 | 0.74889943 | 0.00437807 | LRRC4 | 0.76990857 | 0.04408044 |
| CAV1 | 0.63489736 | 0.00443521 | EPT1 | 0.82795078 | 0.04408619 |
| PNP | 0.67837892 | 0.00444139 | CDC42 | 0.82028228 | 0.04412194 |
| SRSF3 | 0.76672922 | 0.00446884 | NBEAL1 | 0.84458841 | 0.04417812 |
| PLOD2 | 0.77561134 | 0.00450756 | CLTC | 0.83625892 | 0.04423619 |
| ATP6V1A | 0.76889678 | 0.00450756 | KAT2B | 0.80534479 | 0.04435063 |
| A2ML1 | 0.612115 | 0.00451131 | NDFIP2 | 0.83214986 | 0.0444398 |
| ETF1 | 0.75295148 | 0.00452275 | PEX11A | 0.81101355 | 0.04453493 |
| PPP2CA | 0.76256592 | 0.00459161 | NSF | 0.83222465 | 0.04459514 |
| SLC16A4 | 0.69724257 | 0.00459161 | MRPS36 | 0.78965942 | 0.04459514 |
| TPD52L1 | 0.75565633 | 0.00462225 | IFNGR2 | 0.72554575 | 0.04459514 |
| ABI1 | 0.78984533 | 0.00462963 | PPM1D | 0.75457637 | 0.0446064 |
| HSPB8 | 0.54030013 | 0.00463892 | CCDC90B | 0.83348758 | 0.04465495 |
| RAP1A | 0.6286857 | 0.00466577 | KRR1 | 0.8321851 | 0.04472713 |
| UBE2D3 | 0.71948245 | 0.00469068 | S100A2 | 0.55244156 | 0.04472713 |
| ANKRD36BP1 | 0.75516672 | 0.00472447 | SPAST | 0.82037816 | 0.04490377 |
| ZMPSTE24 | 0.78103406 | 0.0047778 | NFYB | 0.80065627 | 0.0449696 |
| EIF4E | 0.7660037 | 0.00485502 | RBM27 | 0.83065796 | 0.04524741 |
| EIF2S1 | 0.77037082 | 0.0048821 | FBXO30 | 0.81207512 | 0.04524741 |
| TIMP3 | 0.595252 | 0.00491633 | C16orf87 | 0.8049152 | 0.04524741 |
| RPS6KB1 | 0.77598677 | 0.0049242 | FUT1 | 0.79442719 | 0.04556648 |
| NMD3 | 0.77550502 | 0.0049698 | SNX27 | 0.81137971 | 0.04590608 |
| ZNF148 | 0.76729032 | 0.00501501 | TGFA | 0.80946531 | 0.04594414 |
| GLRX | 0.72655698 | 0.0050292 | SNAP23 | 0.76908603 | 0.04621429 |
| T0R1AIP2 | 0.75049332 | 0.00505042 | SS18L2 | 0.75904606 | 0.04629091 |
| PDCD10 | 0.77565396 | 0.00508211 | MED13L | 0.80323764 | 0.04639414 |
| MALT1 | 0.75049905 | 0.00508211 | KHDRBS3 | 0.79154107 | 0.04641655 |
| CHD1 | 0.66214755 | 0.00508211 | ZNF165 | 0.76560285 | 0.04651954 |
| XKRX | 0.73215187 | 0.00508311 | RASA2 | 0.77538631 | 0.04658899 |
| SPOPL | 0.67456908 | 0.00509812 | RGS10 | 0.78835868 | 0.04662598 |
| D4S234E | 0.74950027 | 0.0051853 | RPP30 | 0.8120508 | 0.04690347 |
| ZNF217 | 0.7862703 | 0.0052441 | LIPA | 0.83791908 | 0.04694484 |
| C3orf14 | 0.73804789 | 0.00525477 | ZNF438 | 0.62962389 | 0.04694484 |
| ZFX | 0.78085119 | 0.00529941 | LIMCH1 | 0.83370853 | 0.04700596 |
| FAM59A | 0.7610016 | 0.0053185 | LMO7 | 0.82293913 | 0.04710612 |
| LAMTOR3 | 0.75345856 | 0.00532764 | PUS7L | 0.80031465 | 0.04718282 |
| HK2 | 0.78199641 | 0.00534013 | CBFB | 0.82243007 | 0.04719184 |
| GOLT1B | 0.78276656 | 0.0053411 | LMBRD1 | 0.81532931 | 0.04726984 |
| TF | 0.53399053 | 0.00534914 | RIPK2 | 0.69796908 | 0.04754754 |
| SLC12A2 | 0.76713817 | 0.00541558 | SLC36A4 | 0.77616278 | 0.04774991 |
| BLZF1 | 0.76183931 | 0.00543208 | NR4A3 | 0.31905163 | 0.04778283 |
| MORC3 | 0.77320595 | 0.0054433 | TTC13 | 0.79548927 | 0.04780477 |
| ABHD13 | 0.75751055 | 0.0054433 | PRRC1 | 0.84094443 | 0.0480836 |
| ARHGAP10 | 0.76095515 | 0.0055016 | TOMM70A | 0.83565352 | 0.0480836 |
| PPP6C | 0.78390582 | 0.00565944 | EIF4A3 | 0.79211732 | 0.04817496 |
| AKTIP | 0.76242019 | 0.00566109 | FRG1 | 0.7766039 | 0.04833913 |
| IL18 | 0.74117905 | 0.00571372 | DIP2B | 0.81299057 | 0.048344 |
| AMMECR1 | 0.7666803 | 0.00572446 | MRPL50 | 0.83249841 | 0.04843281 |
| SMEK1 | 0.78090529 | 0.0057997 | SHISA9 | 0.76315554 | 0.04871027 |
| NXT2 | 0.76719049 | 0.00584548 | ITGAX | 0.21887106 | 0.0489067 |
| C12orf5 | 0.74487036 | 0.00585798 | FAM120AOS | 0.80855619 | 0.04915381 |
| NFE2L3 | 0.77997497 | 0.00588459 | MAP3K1 | 0.81117229 | 0.04919247 |
| SFIOC2 | 0.76830128 | 0.00591428 | BRMS1L | 0.78256727 | 0.04924817 |
| ERI1 | 0.72854148 | 0.00591448 | ST3GAL5 | 0.81440085 | 0.04925387 |
| ZDHHC20 | 0.78918118 | 0.00595532 | RALBP1 | 0.82325491 | 0.04929206 |
| MS4A7 | 0.50459021 | 0.00595907 | GTPBP10 | 0.83111393 | 0.04933293 |
| CTR9 | 0.77182568 | 0.00597991 | DOCK4 | 0.8068281 | 0.04934341 |
| FAM46A | 0.78379873 | 0.005986 | WDR26 | 0.8064914 | 0.04935751 |
| CPA4 | 0.73474526 | 0.005986 | CTH | 0.74246418 | 0.04943839 |
| TROVE2 | 0.71896413 | 0.00601438 | PARP9 | 0.8069565 | 0.04958092 |
| ARL6IP1 | 0.78399879 | 0.00601695 | ANKHD1 | 0.68180395 | 0.04988035 |
| GADD45A | 0.7103299 | 0.00619164 | TRNT1 | 0.82420431 | 0.04988205 |
| YOD1 | 0.60396183 | 0.00619164 | C15orf48 | 0.66963309 | 0.04988205 |
| CTTNBP2NL | 0.76796852 | 0.00625618 | FERMT2 | 0.80386104 | 0.04991843 |
| PLSCR4 | 0.79632728 | 0.00626049 | REACTOME_IMMUNE_SYSTEM | Genes involved | 1.07E−22 |
| | | | | in Immune | |
| | | | | System | |
| TMEM188 | 0.72279412 | 0.00632262 | REACTOME_METABOLISM_OF_LIP- | Genes involved | 1.47E−18 |
| | | | IDS_AND_LIPOPROTEINS | in Metabolism of | |
| | | | | lipids and | |
| | | | | lipoproteins | |
| MMADHC | 0.78690813 | 0.00643294 | REACTOME_ADAPTIVE_IMMUNE_SYSTEM | Genes involved | 1.46E−15 |
| | | | | in Adaptive | |
| | | | | Immune System | |
| ARG2 | 0.74715273 | 0.00650999 | REACTOME_HEMOSTASIS | Genes involved | 1.57E−14 |
| | | | | in Hemostasis | |
| SLC30A6 | 0.7797098 | 0.00651052 | PID_ERBB1_DOWNSTREAM_PATHWAY | ErbB1 | 2.05E−13 |
| | | | | downstream | |
| | | | | signaling | |
| SPRR2A | 0.37077622 | 0.0065136 | REACTOME_PPARA_ACTIVATES_GENE_EXPRESSION | Genes involved | 1.47E−12 |
| | | | | in PPARA | |
| | | | | Activates Gene | |
| | | | | Expression | |
| SPINK5 | 0.54459219 | 0.00663235 | PID_PDGFRB_PATHWAY | PDGFR-beta | 2.22E−12 |
| | | | | signaling | |
| | | | | pathway | |
| YWHAG | 0.78943324 | 0.00664564 | PID_P53_DOWNSTREAM_PATHWAY | Direct p53 | 8.30E−12 |
| | | | | effectors | |
| IFI16 | 0.78293982 | 0.00669397 | KEGG_PATHWAYS_IN_CANCER | Pathways in | 1.14E−11 |
| | | | | cancer | |
| CYP4F3 | 0.66425151 | 0.00672128 | REACTOME_FATTY_ACID_TRIACYL- | Genes involved | 1.65E−11 |
| | | | GLYCEROL_AND_KETONE_BODY_METABOLISM | in Fatty acid, | |
| | | | | triacylglycerol, | |
| | | | | and ketone body | |
| | | | | metabolism | |
| DSG2 | 0.79997277 | 0.00672627 | NABA_MATRISOME_ASSOCIATED | Ensemble of | 2.28E−10 |
| | | | | genes encoding | |
| | | | | ECM-associated | |
| | | | | proteins including | |
| | | | | ECM-affilaited | |
| | | | | proteins, ECM | |
| | | | | regulators and | |
| | | | | secreted factors | |
| ITGB1 | 0.78721307 | 0.00683767 | REACTOME_TRANSMEMBRANE_TRANS- | Genes involved | 2.48E−09 |
| | | | PORT_OF_SMALL_MOLECULES | in | |
| | | | | Transmembrane | |
| | | | | transport of small | |
| | | | | molecules | |
| SGMS2 | 0.80465915 | 0.00686207 | REACTOME_INNATE_IMMUNE_SYSTEM | Genes involved | 4.47E−09 |
| | | | | in Innate Immune | |
| | | | | System | |
| DMXL2 | 0.75565891 | 0.00687227 | KEGG_REGULATION_OF_ACTIN_CYTOSKELETON | Regulation of | 5.03E−09 |
| | | | | actin cytoskeleton | |
| UGP2 | 0.77377034 | 0.00689688 | KEGG_MAPK_SIGNALING_PATHWAY | MAPK signaling | 6.01E−09 |
| | | | | pathway | |
| TMEM165 | 0.76973779 | 0.00694615 | REACTOME_DIABETES_PATHWAYS | Genes involved | 7.31E−09 |
| | | | | in Diabetes | |
| | | | | pathways | |
| CDC73 | 0.76294135 | 0.00696238 | KEGG_SMALL_CELL_LUNG_CANCER | Small cell lung | 7.31E−09 |
| | | | | cancer | |
| MPP5 | 0.80257658 | 0.00703803 | NABA_ECM_REGULATORS | Genes encoding | 7.31E−09 |
| | | | | enzymes and | |
| | | | | their regulators | |
| | | | | involved in the | |
| | | | | remodeling of the | |
| | | | | extracellular | |
| | | | | matrix | |
| SP1 | 0.76405586 | 0.00705511 | REACTOME_APOPTOSIS | Genes involved | 7.61E−09 |
| | | | | in Apoptosis | |
| VDAC2 | 0.76968598 | 0.00707017 | NABA_MATRISOME | Ensemble of | 1.09E−08 |
| | | | | genes encoding | |
| | | | | extracellular | |
| | | | | matrix and | |
| | | | | extracellular | |
| | | | | matrix-associated | |
| | | | | proteins | |
| LRRFIP1 | 0.77118612 | 0.0070728 | PID_NFKAPPAB_CANONICAL_PATHWAY | Canonical NF- | 1.11E−08 |
| | | | | kappaB pathway | |
| C14orfl28 | 0.71927857 | 0.00711871 | KEGG_APOPTOSIS | Apoptosis | 1.29E−08 |
| LYPD3 | 0.68004615 | 0.00715007 | REACTOME_CLASS_I_MHC_MEDIATED_ANTI- | Genes involved | 1.98E−08 |
| | | | GEN_PROCESSING_PRESENTATION | in Class I MHC | |
| | | | | mediated antigen | |
| | | | | processing & | |
| | | | | presentation | |
| PTPRZ1 | 0.78817053 | 0.00719019 | REACTOME_TOLL_RECEPTOR_CASCADES | Genes involved | 2.71E−08 |
| | | | | in Toll Receptor | |
| | | | | Cascades | |
| RAB18 | 0.76366275 | 0.00722127 | REACTOME_ACTIVATED_TLR4_SIGNALLING | Genes involved | 2.71E−08 |
| | | | | in Activated | |
| | | | | TLR4 signalling | |
| AP3S1 | 0.75774232 | 0.00729569 | PID_CDC42_PATHWAY | CDC42 signaling | 2.71E−08 |
| | | | | events | |
| C17orf91 | 0.74332375 | 0.00730188 | KEGG_NOD_LIKE_RECEPTOR_SIGNALING_PATHWAY | NOD-like | 4.69E−08 |
| | | | | receptor signaling | |
| | | | | pathway | |
| XIAP | 0.79828911 | 0.0073532 | KEGG_FOCAL_ADHESION | Focal adhesion | 7.43E−08 |
| L0C374443 | 0.71361722 | 0.00737354 | REACTOME_TRAF6_MEDIATED_INDUC- | Genes involved | 9.93E−08 |
| | | | TION_OF_NFKB_AND_MAP_KINASES_UP- | in TRAF6 | |
| | | | ON_TLR7_8_OR_9_ACTIVATION | mediated | |
| | | | | induction of | |
| | | | | NFkB and MAP | |
| | | | | kinases upon | |
| | | | | TLR7/8 or 9 | |
| | | | | activation | |
| TWF1 | 0.79895735 | 0.00742683 | PID_TNF_PATHWAY | TNF receptor | 1.12E−07 |
| | | | | signaling | |
| | | | | pathway | |
| ELF1 | 0.77273855 | 0.00744917 | KEGG_EPITHELIAL_CELL_SIGNALING_IN_HELICO- | Epithelial cell | 1.49E−07 |
| | | | BACTER_PYLORI_INFECTION | signaling in | |
| | | | | Helicobacter | |
| | | | | pylori infection | |
| S100A14 | 0.76635669 | 0.00744917 | BIOCARTA_HIVNEF_PATHWAY | HIV-I Nef: | 1.71E−07 |
| | | | | negative effector | |
| | | | | of Fas and TNF | |
| SLC16A6 | 0.70750259 | 0.00745345 | KEGG_P53_SIGNALING_PATHWAY | p53 signaling | 1.71E−07 |
| | | | | pathway | |
| DCUN1D3 | 0.56968422 | 0.00747439 | REACTOME_ANTIGEN_PROCESSING_UBIQUI- | Genes involved | 1.79E−07 |
| | | | TINATION_PROTEASOME_DEGRADATION | in Antigen | |
| | | | | processing: | |
| | | | | Ubiquitination & | |
| | | | | Proteasome | |
| | | | | degradation | |
| SLC44A2 | 0.76320925 | 0.00753544 | PID_AP1_PATHWAY | AP-1 | 1.93E−07 |
| | | | | transcription | |
| | | | | factor network | |
| SESTD1 | 0.7924907 | 0.00756289 | KEGG_PATHOGENIC_ESCHERICHIA_COLI_INFECTION | Pathogenic | 1.93E−07 |
| | | | | Escherichia coli | |
| | | | | infection | |
| S100P | 0.64809558 | 0.00767001 | REACTOME_MYD88_MAL_CASCADE_INITI- | Genes involved | 2.31E−07 |
| | | | ATED_ON_PLASMA_MEMBRANE | in MyD88: Mal | |
| | | | | cascade initiated | |
| | | | | on plasma | |
| | | | | membrane | |
| ARPP19 | 0.78635202 | 0.00768701 | REACTOME_SIGNALLING_BY_NGF | Genes involved | 2.51E−07 |
| | | | | in Signalling by | |
| | | | | NGF | |
| KLF10 | 0.76312973 | 0.00775452 | KEGG_UBIQUITIN_MEDIATED_PROTEOLYSIS | Ubiquitin | 2.51E−07 |
| | | | | mediated | |
| | | | | proteolysis | |
| TGM1 | 0.55760183 | 0.00777418 | REACTOME_CYTOKINE_SIGNAL- | Genes involved | 2.56E−07 |
| | | | ING_IN_IMMUNE_SYSTEM | in Cytokine | |
| | | | | Signaling in | |
| | | | | Immune system | |
| BHLHE40 | 0.78959699 | 0.00777685 | KEGG_NEUROTROPHIN_SIGNALING_PATHWAY | Neurotrophin | 3.27E−07 |
| | | | | signaling | |
| | | | | pathway | |
| PLBD1 | 0.70356721 | 0.00777685 | REACTOME_TRIF_MEDIATED_TLR3_SIGNALING | Genes involved | 3.49E−07 |
| | | | | in TRIF mediated | |
| | | | | TLR3 signaling | |
| MYC | 0.76472327 | 0.00781167 | BIOCARTA_MAPK_PATHWAY | MAPKinase | 3.88E−07 |
| | | | | Signaling | |
| | | | | Pathway | |
| FAM91A1 | 0.77751938 | 0.00785683 | REACTOME_MEMBRANE_TRAFFICKING | Genes involved | 4.44E−07 |
| | | | | in Membrane | |
| | | | | Trafficking | |
| MREG | 0.76267651 | 0.00794736 | BIOCARTA_SALMONELLA_PATHWAY | How does | 4.71E−07 |
| | | | | salmonella hijack | |
| | | | | a cell | |
| GDPD1 | 0.81908069 | 0.0079732 | PID_HIF1_TFPATHWAY | HIF-1-alpha | 6.39E−07 |
| | | | | transcription | |
| | | | | factor network | |
| GPD2 | 0.80071021 | 0.00805078 | PID_TGFBR_PATHWAY | TGF-beta | 6.45E−07 |
| | | | | receptor signaling | |
| PVRL4 | 0.77402462 | 0.00805078 | PID_MYC_ACTIV_PATHWAY | Validated targets | 7.35E−07 |
| | | | | ofC-MYC | |
| | | | | transcriptional | |
| | | | | activation | |
| SUCLA2 | 0.76523468 | 0.00805078 | BIOCARTA_ACTINY_PATHWAY | Y branching of | 7.40E−07 |
| | | | | actin filaments | |
| ACER3 | 0.77959865 | 0.00808456 | REACTOME_PHOSPHOLIPID_METABOLISM | Genes involved | 7.42E−07 |
| | | | | in Phospholipid | |
| | | | | metabolism | |
| RABL3 | 0.7748714 | 0.00809777 | PID_MET_PATHWAY | Signaling events | 8.18E−07 |
| | | | | mediated by | |
| | | | | Hepatocyte | |
| | | | | Growth Factor | |
| | | | | Receptor (c-Met) | |
| RAB10 | 0.79901305 | 0.0082063 | KEGG_ENDOCYTOSIS | Endocytosis | 8.35E−07 |
| PJA2 | 0.7769656 | 0.00823489 | REACTOME_INSULIN_SYNTHESIS_AND_PROCESSING | Genes involved | 1.08E−06 |
| | | | | in Insulin | |
| | | | | Synthesis and | |
| | | | | Processing | |
| CAP1 | 0.72655632 | 0.00826187 | KEGG_PANCREATIC_CANCER | Pancreatic cancer | 1.12E−06 |
| RDX | 0.80715808 | 0.00827579 | KEGG_RENAL_CELLCARCINOMA | Renal cell | 1.12E−06 |
| | | | | carcinoma | |
| TES | 0.79507705 | 0.00829307 | PID_ATF2_PATHWAY | ATF-2 | 1.25E−06 |
| | | | | transcription | |
| | | | | factor network | |
| MUDENG | 0.79933934 | 0.0083017 | REACTOME_SLC_MEDIATED_TRANS- | Genes involved | 1.30E−06 |
| | | | MEMBRANE_TRANSPORT | in SLC-mediated | |
| | | | | transmembrane | |
| | | | | transport | |
| PPIL3 | 0.76235604 | 0.00834263 | REACTOME_SIGNAL- | Genes involved | 1.40E−06 |
| | | | ING_BY_THE_B_CELL_RECEPTOR_BCR | in Signaling by | |
| | | | | the B Cell | |
| | | | | Receptor (BCR) | |
| BIRC2 | 0.78625068 | 0.00837842 | PID_FOXO_PATHWAY | FoxO family | 1.45E−06 |
| | | | | signaling | |
| CCNB1 | 0.7807843 | 0.00847331 | REACTOME_NFKB_AND_MAP_KINASES_ACTI- | Genes involved | 1.46E−06 |
| | | | VATION_MEDIATED_BY_TLR4_SIGNAL- | in NFkB and | |
| | | | ING_REPERTOIRE | MAP kinases | |
| | | | | activation | |
| | | | | mediated by | |
| | | | | TLR4 signaling | |
| | | | | repertoire | |
| ATL2 | 0.77916813 | 0.0084764 | REACTOME_PLATELET_ACTIVATION_SIGNALING | Genes involved | 1.48E−06 |
| | | | AND_AGGREGATION | in Platelet | |
| | | | | activation, | |
| | | | | signaling and | |
| | | | | aggregation | |
| SORD | 0.75801895 | 0.0084879 | KEGG_TGF_BETA_SIGNALING_PATHWAY | TGF-beta | 1.74E−06 |
| | | | | signaling | |
| | | | | pathway | |
| ATP11C | 0.79291526 | 0.00853151 | PID_EPHB_FWD_PATHWAY | EPHB forward | 1.77E−06 |
| | | | | signaling | |
| RRAGC | 0.75615041 | 0.00853151 | REACTOME_APOPTOTIC_CLEA- | Genes involved | 1.77E−06 |
| | | | VAGE_OF_CELLULAR_PROTEINS | in Apoptotic | |
| | | | | cleavage of | |
| | | | | cellular proteins | |
| IFNGR1 | 0.69711126 | 0.00853151 | BIOCARTA_CDC42RAC_PATHWAY | Role of PI3K | 2.02E−06 |
| | | | | subunit p85 in | |
| | | | | regulation of | |
| | | | | Actin | |
| | | | | Organization and | |
| | | | | Cell Migration | |
| STEAP2 | 0.78974481 | 0.00856925 | REACTOME_CELL_CYCLE_MITOTIC | Genes involved | 2.04E−06 |
| | | | | in Cell Cycle, | |
| | | | | Mitotic | |
| WDR72 | 0.64839931 | 0.0086094 | PID_CASPASE_PATHWAY | Caspase cascade | 2.45E−06 |
| | | | | in apoptosis | |
| KRT4 | 0.67492283 | 0.00863552 | REACTOME_CIRCADIAN_CLOCK | Genes involved | 2.97E−06 |
| | | | | in Circadian | |
| | | | | Clock | |
| HS2ST1 | 0.7871526 | 0.00868303 | ST_FAS_SIGNALING_PATHWAY | Fas Signaling | 3.14E−06 |
| | | | | Pathway | |
| ZCCHC10 | 0.75926787 | 0.00868842 | BIOCARTA_DEATH_PATHWAY | Induction of | 3.18E−06 |
| | | | | apoptosis through | |
| | | | | DR3 and DR4/5 | |
| | | | | Death Receptors | |
| PPP2R2A | 0.79190305 | 0.00877521 | PID_RAC1_PATHWAY | RAC1 signaling | 3.49E−06 |
| | | | | pathway | |
| SQRDL | 0.75607401 | 0.00879068 | SIG_PIP3_SIGNALING_IN_CARDIAC_MYOCTES | Genes related to | 4.27E−06 |
| | | | | PIP3 signaling in | |
| | | | | cardiac myocytes | |
| STK38 | 0.78754071 | 0.00886943 | PID_BETA_CATENIN_NUC_PATHWAY | Regulation of | 4.37E−06 |
| | | | | nuclear beta | |
| | | | | catenin signaling | |
| | | | | and target gene | |
| | | | | transcription | |
| LYRM1 | 0.7382844 | 0.00898135 | REACTOME_APOPTOTIC_CLEA- | Genes involved | 5.72E−06 |
| | | | VAGE_OF_CELL_ADHESION_PROTEINS | in Apoptotic | |
| | | | | cleavage of cell | |
| | | | | adhesion | |
| | | | | proteins | |
| SYK | 0.64957988 | 0.00898135 | PID-PLK1_PATHWAY | PLK1 signaling | 6.25E−06 |
| | | | | events | |
| S100A10 | 0.76365242 | 0.00900115 | REACTOME_METABOLISM_OF_PROTEINS | Genes involved | 6.47E−06 |
| | | | | in Metabolism of | |
| | | | | proteins | |
| NTS | 0.73291849 | 0.00900309 | REACTOME_BMAL1_CLOCK_NPAS2_ACTI- | Genes involved | 6.56E−06 |
| | | | VATES_CIRCADIAN_EXPRESSION | in | |
| | | | | BMAL1: CLOCK/ | |
| | | | | NPAS2 | |
| | | | | Activates | |
| | | | | Circadian | |
| | | | | Expression | |
| LOC440434 | 0.68882777 | 0.00901276 | ST_P38_MAPK_PATHWAY | p38 MAPK | 8.35E−06 |
| | | | | Pathway | |
| GNA13 | 0.63583346 | 0.00908917 | REACTOME_DEVELOPMENTAL_BIOLOGY | Genes involved | 9.75E−06 |
| | | | | in Developmental | |
| | | | | Biology | |
| STK17A | 0.73661542 | 0.00912019 | PID_ARF6_TRAFFICKING_PATHWAY | Arf6 trafficking | 1.10E−05 |
| | | | | events | |
| ITSN2 | 0.76584981 | 0.00913286 | ST_TUMOR_NECROSIS_FACTOR_PATHWAY | Tumor Necrosis | 1.23E−05 |
| | | | | Factor Pathway. | |
| GOLT1A | 0.71280825 | 0.00924664 | PID_ECADHERIN_NASCENT_AJ_PATHWAY | E-cadherin | 1.29E−05 |
| | | | | signaling in the | |
| | | | | nascent adherens | |
| | | | | junction | |
| DIAPH1 | 0.77552848 | 0.00932056 | REACTOME_MAP_KINASE_ACTI- | Genes involved | 1.29E−05 |
| | | | VATION_IN_TLR_CASCADE | in MAP kinase | |
| | | | | activation in TLR | |
| | | | | cascade | |
| ZNF654 | 0.74649612 | 0.00934308 | KEGG_B_CELL_RECEPTOR_SIGNALING_PATHWAY | B cell receptor | 1.31E−05 |
| | | | | signaling | |
| | | | | pathway | |
| FPR3 | 0.48825296 | 0.00934423 | BIOCARTA_MITOCHONDRIA_PATHWAY | Role of | 1.40E−05 |
| | | | | Mitochondria in | |
| | | | | Apoptotic | |
| | | | | Signaling | |
| RCHY1 | 0.79749711 | 0.00935 | REACTOME_SIGNAL- | Genes involved | 1.48E−05 |
| | | | ING_BY_TGF_BETA_RECEPTOR_COMPLEX | in Signaling by | |
| | | | | TGF-beta | |
| | | | | Receptor | |
| | | | | Complex | |
| 4-Mar | 0.77086317 | 0.00935 | SIG_INSULIN_RECEPTOR_PATH- | Genes related to | 1.49E−05 |
| | | | WAY_IN_CARDIAC_MYOCYTES | the insulin | |
| | | | | receptor pathway | |
| REEP3 | 0.8126155 | 0.0094555 | REACTOME_NOD1_2_SIGNALING_PATHWAY | Genes involved | 1.49E−05 |
| | | | | in NOD1/2 | |
| | | | | Signaling | |
| | | | | Pathway | |
| TFG | 0.79338065 | 0.00956122 | ST_JNK_MAPK_PATHWAY | JNK MAPK | 1.49E−05 |
| | | | | Pathway | |
| SNX18 | 0.76111449 | 0.00960834 | REACTOME_MITOTIC_G1_G1_S_PHASES | Genes involved | 1.59E−05 |
| | | | | in Mitotic G1- | |
| | | | | G1/S phases | |
| TMEM79 | 0.77640651 | 0.00962273 | REACTOME_NGF_SIGNAL- | Genes involved | 1.59E−05 |
| | | | LING_VIA_TRKA_FROM_THE_PLASMA_MEMBRANE | in NGF signalling | |
| | | | | via TRKA from | |
| | | | | the plasma | |
| | | | | membrane | |
| C12orf35 | 0.56826344 | 0.00962273 | REACTOME_ACTIVA- | Genes involved | 1.63E−05 |
| | | | TION_OF_NF_KAPPAB_IN_B_CELLS | in Activation of | |
| | | | | NF-kappaB in B | |
| | | | | Cells | |
| GOLGA4 | 0.8023233 | 0.00962569 | PID_AVB3_OPN_PATHWAY | Osteopontin- | 1.85E−05 |
| | | | | mediated events | |
| PLA2R1 | 0.78448235 | 0.00972618 | PID_CD40_PATHWAY | CD40/CD40L | 1.85E−05 |
| | | | | signaling | |
| SYPL1 | 0.80241463 | 0.00979309 | PID_RB_1PATHWAY | Regulation of | 1.86E−05 |
| | | | | retinoblastoma | |
| | | | | protein | |
| C15orf34 | 0.76100423 | 0.0098085 | PID_TAP63_PATHWAY | Validated | 2.31E−05 |
| | | | | transcriptional | |
| | | | | targets of TAp63 | |
| | | | | isoforms | |
| AGA | 0.77317636 | 0.00987069 | REACTOME_APOPTOTIC_EXECUTION_PHASE | Genes involved | 2.31E−05 |
| | | | | in Apoptotic | |
| | | | | execution phase | |
| 10-Sep | 0.80194663 | 0.00988696 | ST_ERK1_ERK2_MAPK_PATHWAY | ERK1/ERK2 | 2.31E−05 |
| | | | | MAPK Pathway | |
| MFAP3 | 0.78771375 | 0.00994587 | BIOCARTA_CASPASE_PATHWAY | Caspase Cascade | 2.41E−05 |
| | | | | in Apoptosis | |
| | | | PID_INTEGRIN3_PATHWAY | Beta3 integrin | 2.55E−05 |
| | | | | cell surface | |
| | | | | interactions | |

TABLE 3
List of known asthma-associated genes³⁷ that overlap with genes in the RNAseq data sets.

| Number | |
|---|---|
| of Genes | Genes |
| 70 | ACE; ACO1; ACP1; ADRB2; ALOX5; C11orf71; C3; C3AR1; C5orf56; CCL5; |
| | CCR5; CD14; CDK2; CFTR; CHML; CRCT1; CYFIP2; DAP3; DEFB1; DENND1B; |
| | GAB1; GATA3; GSDMB; GSTP1; GSTT1; HAVCR2; HLA-DOA; HLA-DPA1; |
| | HLA-DPB1; HLA-DQA1; HLA-DQB1; HLA-DRA; HLA-DRB1; HNMT; IKZF4; |
| | IL15; IL18; IL1B; IL1R1; IL1RN; IL2RB; IL33; IL5RA; IL6R; IL8; IRAK2; IRF1; |
| | NDFIP1; NOD1; OPN3; ORMDL3; PBX2; PCDH20; PDE4D; PHF11; RAD50; |
| | RORA; SERPINA3; SLC22A5; SMAD3; SPATS2L; SPINK5; STAT6; TAP1; |
| | TGFB1; TIMP1; TLE4; TLR2; TLR4; VDR |

TABLE 4
List of the genes identified in the eight classification
models and unique genes comprising the asthma gene panel.

| Model/Asthma | Number | | Optimal Classification |
|---|---|---|---|
| Panel subset | of Genes | Genes | Threshold |
| LR-RFE & | 90 | PCSK6, HIPK2, TXNDC5, B3GNT6, CD177, | Approx 0.76 |
| Logistic | | KRT24, FCGBP, DLEC1, SERPINB3, CLEC2B, | |
| | | PTER, ERAP2, SYNM, CDKN1A, SPRR1A, | |
| | | C12orf36, SERPINE2, XIST, SLC9A3, SCD, | |
| | | TEKT2, EPPK1, RPH3AL, MS4A8B, SDK1, | |
| | | IGF1, FOS, SERPINB11, CPA3, HLA.C, | |
| | | SLC26A4, CYP1B1, SCGB1A1, SEMA5A, ESR1, | |
| | | CDHR3, NWD1, TMEM190, GNAL, ZNF117, | |
| | | EPDR1, DEFB1, PTAFR, SPRR2D, CHCHD10, | |
| | | LOC90784, AKR1B15, CROCCP2, S100A8, | |
| | | TFPI, C3, S100A7, DUSP1, LY6D, SORD, | |
| | | SERPINF1, TPSB2, NMU, GSTT1, LPAR6, | |
| | | CYFIP2, CPAMD8, SLC5A8, SLC5A3, SC4MOL, | |
| | | NR1D1, ARL4D, ALDH1A3, LPHN1, | |
| | | LOC286002, CRABP2, CEBPD, C6orf105, | |
| | | TM4SF1, ANKRD9, PCP4L1, SLC35E2, | |
| | | LOC388564, DNAI1, SLC44A5, LTBP1, CROCC, | |
| | | NCRNA00152, CDH26, TPSAB1, RHCG, | |
| | | CLEC7A, IER3, MMP9, ALOX15B | |
| LR-RFE & | 90 | PCSK6, HIPK2, TXNDC5, B3GNT6, CD177, | Approx 0.52 |
| SVM-Linear | | KRT24, FCGBP, DLEC1, SERPINB3, CLEC2B, | |
| | | PTER, ERAP2, SYNM, CDKN1A, SPRR1A, | |
| | | C12orf36, SERPINE2, XIST, SLC9A3, SCD, | |
| | | TEKT2, EPPK1, RPH3AL, MS4A8B, SDK1, | |
| | | IGF1, FOS, SERPINB11, CPA3, HLA.C, | |
| | | SLC26A4, CYP1B1, SCGB1A1, SEMA5A, ESR1, | |
| | | CDHR3, NWD1, TMEM190, GNAL, ZNF117, | |
| | | EPDR1, DEFB1, PTAFR, SPRR2D, CHCHD10, | |
| | | LOC90784, AKR1B15, CROCCP2, S100A8, | |
| | | TFPI, C3, S100A7, DUSP1, LY6D, SORD, | |
| | | SERPINF1, TPSB2, NMU, GSTT1, LPAR6, | |
| | | CYFIP2, CPAMD8, SLC5A8, SLC5A3, SC4MOL, | |
| | | NR1D1, ARL4D, ALDH1A3, LPHN1, | |
| | | LOC286002, CRABP2, CEBPD, C6orf105, | |
| | | TM4SF1, ANKRD9, PCP4L1, SLC35E2, | |
| | | LOC388564, DNAI1, SLC44A5, LTBP1, CROCC, | |
| | | NCRNA00152, CDH26, TPSAB1, RHCG, | |
| | | CLEC7A, IER3, MMP9, ALOX15B | |
| SVM-RFE & | 119 | PYCR1, TXNDC5, B3GNT6, CD177, FAM46C, | Approx 0.64 |
| SVM-Linear | | PPP2R2C, VWA1, PTER, KAL1, GNG4, ERAP2, | |
| | | SYNM, CCL5, TRIM31, DOCK1, NFKBIZ, | |
| | | MGST1, SPRR1A, PLIN4, TNFRSF18, ISYNA1, | |
| | | SLC9A4, SLC9A2, SLC9A3, CPA3, SERPINB11, | |
| | | OSM, MSMB, LGALS9C, SDK1, G0S2, | |
| | | DPYSL3, RPH3AL, KIF7, C11orf9, COL1A1, | |
| | | HLA.C, HCAR2, SLC26A4, SHF, SERPINF1, | |
| | | SPRR2D, SCGB1A1, ZDHHC2, SEMA5A, ESR1, | |
| | | VAV2, NWD1, CYP2E1, KRT13, KRT10, GNAL, | |
| | | ZNF117, EPDR1, PAX3, KLHL29, NBPF1, | |
| | | GPNMB, FABP5, CLCA2, C7orf13, SPRR2F, | |
| | | LOC90784, CYP2B6, CROCCP2, TFPI, S100A7, | |
| | | DUSP1, LY6D, PHYHD1, SORD, TMEM64, | |
| | | C15orf48, MXRA8, IL4I1, TPSB2, NMU, | |
| | | BPIFA2, ZNF528, HTR3A, STEAP1, STEAP2, | |
| | | LPAR6, OBSCN, MT2A, CPAMD8, D4S234E, | |
| | | ECM1, SLC16A4, LRRC26, CRCT1, SLC5A5, | |
| | | ZC3H12A, NR1D1, ALDH1A3, SLC37A2, | |
| | | LPHN1, CRABP2, TM4SF1, ANKRD9, CXCR7, | |
| | | TF, TMEM220, LOC388564, XIST, SLC44A5, | |
| | | LTBP1, RAB3B, MEX3D, TPSAB1, RHCG, | |
| | | SRRM3, SCGB3A1, RND1, REC8, SCD, | |
| | | ALOX15B, ATP6V0E2, COL6A6 | |
| SVM-RFE & | 119 | PYCR1, TXNDC5, B3GNT6, CD177, FAM46C, | Approx 0.69 |
| Logistic | | PPP2R2C, VWA1, PTER, KAL1, GNG4, ERAP2, | |
| | | SYNM, CCL5, TRIM31, DOCK1, NFKBIZ, | |
| | | MGST1, SPRR1A, PLIN4, TNFRSF18, ISYNA1, | |
| | | SLC9A4, SLC9A2, SLC9A3, CPA3, SERPINB11, | |
| | | OSM, MSMB, LGALS9C, SDK1, G0S2, | |
| | | DPYSL3, RPH3AL, KIF7, C11orf9, COL1A1, | |
| | | HLA.C, HCAR2, SLC26A4, SHF, SERPINF1, | |
| | | SPRR2D, SCGB1A1, ZDHHC2, SEMA5A, ESR1, | |
| | | VAV2, NWD1, CYP2E1, KRT13, KRT10, GNAL, | |
| | | ZNF117, EPDR1, PAX3, KLHL29, NBPF1, | |
| | | GPNMB, FABP5, CLCA2, C7orf13, SPRR2F, | |
| | | LOC90784, CYP2B6, CROCCP2, TFPI, S100A7, | |
| | | DUSP1, LY6D, PHYHD1, SORD, TMEM64, | |
| | | C15orf48, MXRA8, IL4I1, TPSB2, NMU, | |
| | | BPIFA2, ZNF528, HTR3A, STEAP1, STEAP2, | |
| | | LPAR6, OBSCN, MT2A, CPAMD8, D4S234E, | |
| | | ECM1, SLC16A4, LRRC26, CRCT1, SLC5A5, | |
| | | ZC3H12A, NR1D1, ALDH1A3, SLC37A2, | |
| | | LPHN1, CRABP2, TM4SF1, ANKRD9, CXCR7, | |
| | | TF, TMEM220, LOC388564, XIST, SLC44A5, | |
| | | LTBP1, RAB3B, MEX3D, TPSAB1, RHCG, | |
| | | SRRM3, SCGB3A1, RND1, REC8, SCD, | |
| | | ALOX15B, ATP6V0E2, COL6A6 | |
| LR-RFE & | 90 | PCSK6, HIPK2, TXNDC5, B3GNT6, CD177, | Approx 0.49 |
| AdaBoost | | KRT24, FCGBP, DLEC1, SERPINB3, CLEC2B, | |
| | | PTER, ERAP2, SYNM, CDKN1A, SPRR1A, | |
| | | C12orf36, SERPINE2, XIST, SLC9A3, SCD, | |
| | | TEKT2, EPPK1, RPH3AL, MS4A8B, SDK1, | |
| | | IGF1, FOS, SERPINB11, CPA3, HLA.C, | |
| | | SLC26A4, CYP1B1, SCGB1A1, SEMA5A, ESR1, | |
| | | CDHR3, NWD1, TMEM190, GNAL, ZNF117, | |
| | | EPDR1, DEFB1, PTAFR, SPRR2D, CHCHD10, | |
| | | LOC90784, AKR1B15, CROCCP2, S100A8, | |
| | | TFPI, C3, S100A7, DUSP1, LY6D, SORD, | |
| | | SERPINF1, TPSB2, NMU, GSTT1, LPAR6, | |
| | | CYFIP2, CPAMD8, SLC5A8, SLC5A3, SC4MOL, | |
| | | NR1D1, ARL4D, ALDH1A3, LPHN1, | |
| | | LOC286002, CRABP2, CEBPD, C6orf105, | |
| | | TM4SF1, ANKRD9, PCP4L1, SLC35E2, | |
| | | LOC388564, DNAI1, SLC44A5, LTBP1, CROCC, | |
| | | NCRNA00152, CDH26, TPSAB1, RHCG, | |
| | | CLEC7A, IER3, MMP9, ALOX15B | |
| LR-RFE & | 90 | PCSK6, HIPK2, TXNDC5, B3GNT6, CD177, | Approx 0.60 |
| RandomForest | | KRT24, FCGBP, DLEC1, SERPINB3, CLEC2B, | |
| | | PTER, ERAP2, SYNM, CDKN1A, SPRR1A, | |
| | | C12orf36, SERPINE2, XIST, SLC9A3, SCD, | |
| | | TEKT2, EPPK1, RPH3AL, MS4A8B, SDK1, | |
| | | IGF1, FOS, SERPINB11, CPA3, HLA.C, | |
| | | SLC26A4, CYP1B1, SCGB1A1, SEMA5A, ESR1, | |
| | | CDHR3, NWD1, TMEM190, GNAL, ZNF117, | |
| | | EPDR1, DEFB1, PTAFR, SPRR2D, CHCHD10, | |
| | | LOC90784, AKR1B15, CROCCP2, S100A8, | |
| | | TFPI, C3, S100A7, DUSP1, LY6D, SORD, | |
| | | SERPINF1, TPSB2, NMU, GSTT1, LPAR6, | |
| | | CYFIP2, CPAMD8, SLC5A8, SLC5A3, SC4MOL, | |
| | | NR1D1, ARL4D, ALDH1A3, LPHN1, | |
| | | LOC286002, CRABP2, CEBPD, C6orf105, | |
| | | TM4SF1, ANKRD9, PCP4L1, SLC35E2, | |
| | | LOC388564, DNAI1, SLC44A5, LTBP1, CROCC, | |
| | | NCRNA00152, CDH26, TPSAB1, RHCG, | |
| | | CLEC7A, IER3, MMP9, ALOX15B | |
| SVM-RFE & | 123 | HSPA6, GSTA1, PLIN4, TXNDC5, B3GNT6, | Approx 0.50 |
| RandomForest | | BHLHE40, CYP4F11, CD177, IRX5, TMX4, | |
| | | DDIT4, SCCPDH, FCGBP, ARRDC4, MUC16, | |
| | | TSPAN8, ACOT2, SPINK5, C19orf51, PTER, | |
| | | F2R, GNG4, SERPING1, C14orf167, ERAP2, | |
| | | MMP10, DOCK1, NFKBIZ, CHCHD10, MGST1, | |
| | | C12orf36, CLCA2, XIST, SLC9A2, SLC9A3, | |
| | | CPA3, TEKT2, EPPK1, SERPINB11, OVCA2, | |
| | | MSMB, CDC25B, TNS3, SDK1, FOS, RPH3AL, | |
| | | KIF7, COL1A1, HLA.C, HCAR2, SLC26A4, | |
| | | PAX3, SERPINF1, SPRR2F, DNER, GSTT1, | |
| | | ESR1, VAV2, CYP2E1, TMEM190, KRT13, | |
| | | GNAL, RPSAP58, FABP5, MALAT1, C7orf13, | |
| | | SCGB1A1, AKR1B15, CYP2B6, HBEGF, TFPI, | |
| | | C3, S100A7, DUSP1, HERC2P2, SORD, | |
| | | C15orf48, MXRA8, IL4I1, TPSB2, NMU, | |
| | | SEMA5A, BPIFA2, PRSS3, AK4, BASP1, | |
| | | HTR3A, COL21A1, LPAR6, MKI67, CYFIP2, | |
| | | CPAMD8, D4S234E, CRCT1, MFSD6L, CIT, | |
| | | SLC5A8, NR1D1, ALDH1A3, SLC37A2, LPHN1, | |
| | | LOC286002, CRABP2, CEBPD, ANKRD9, | |
| | | CXCR7, SLC35E2, LOC388564, SLC9A4, | |
| | | SLC44A5, LTBP1, CRYM, RAB3B, KAL1, | |
| | | MEX3D, TPSAB1, NCRNA00086, HLA.DQA1, | |
| | | RHCG, REC8, ALOX15B, ATP6V0E2, COL6A6 | |
| SVM-RFE & | 212 | IDAS, NR1D1, HIPK2, RCBTB2, PYCR1, | Approx 0.55 |
| AdaBoost | | TSPAN8, CPPED1, B3GNT6, HLA.DPB1, | |
| | | PARD6G, IP6K3, EIF1AX, CD177, FAM46C, | |
| | | IRX5, C3orf14, IFITM1, NGEF, SCCPDH, | |
| | | PPP2R2C, XYLT1, DLEC1, MUC16, SERPINB3, | |
| | | ACOT2, SLC35E2, SMPDL3B, C19orf51, | |
| | | LOC388796, MPV17L, SYK, SLC9A4, PTER, | |
| | | F2R, GNG4, BST1, C14orf167, CCNO, ERAP2, | |
| | | SYNM, EVL, CCL5, TRIM31, DOCK1, RRAS, | |
| | | MALAT1, MGST1, SLC29A1, C12orf36, PLIN4, | |
| | | SERPINE2, JUB, PTN, SLC9A2, CLEC7A, | |
| | | CPA3, TEKT2, EPPK1, SERPINB11, OVCA2, | |
| | | OSM, VWA1, CDC25B, LGALS9C, MS4A8B, | |
| | | SDK1, S100A13, DPYSL3, PDLIM2, RPH3AL, | |
| | | KIF7, C11orf9, TEKT4P2, PMEPA1, HLA.C, | |
| | | HCAR2, SLC26A4, PAX3, NLRP1, GIMAP6, | |
| | | SPRR2F, SPRR2C, DNER, ABCG1, ZDHHC2, | |
| | | ZNF532, SEMA5A, ESR1, VAV2, NWD1, | |
| | | CYP2E1, TMEM190, MAOB, CXCR7, GNAL, | |
| | | ZNF117, GAS7, EPDR1, NCF2, DEFB1, | |
| | | H2AFY2, GRTP1, NBPF1, CROCCP2, | |
| | | SERPING1, KRT5, CHCHD10, TP63, C7orf13, | |
| | | SCGB1A1, LOC90784, HIC1, AKR1B15, | |
| | | GAS2L2, HIFX, CYP2B6, GPNMB, HBEGF, | |
| | | ACAT2, TFPI, C3, S100A7, DUSP1, SLC9A3, | |
| | | LYSMD2, HERC2P2, PHYHD1, TOP1MT, | |
| | | PLCL2, SORD, TMEM64, C15orf48, PLXND1, | |
| | | CD8A, MXRA8, IL4I1, IL2RB, NMU, GSTT1, | |
| | | BPIFA2, ZNF528, IL32, WDR96, NPNT, | |
| | | DMRTA2, BASP1, CEBPD, HTR3A, COL21A1, | |
| | | OBSCN, CYFIP2, CPAMD8, XIST, D4S234E, | |
| | | IGF1R, ECM1, PTPRZ1, CRCT1, RRM2, MLKL, | |
| | | CIT, SC4MOL, DDIT4, ELF5, ARL4D, | |
| | | ALDH1A3, SLC37A2, LPHN1, LOC286002, | |
| | | CRABP2, CCNJL, MEGF6, TM4SF1, ANKRD9, | |
| | | C8orf4, SLC16A14, ALOX15B, PCP4L1, TOR1B, | |
| | | TF, ACOT11, HOMER3, LOC388564, CYP1B1, | |
| | | DNAI1, LRP12, LTBP1, ANXA6, CARD11, | |
| | | CROCC, CES1, ALDH3B2, NCRNA00152, | |
| | | RAB3B, TNC, KAL1, FOXN4, MEX3D, FCGBP, | |
| | | TPSAB1, NCRNA00086, HLA.DOA, KRT78, | |
| | | RHCG, NCALD, REC8, RDH10, SERPINF1, | |
| | | ATP6V0E2, POLR2J3, POU2F3, TCTEX1D4 | |
| Asthma gene | 275 | IDAS, HSPA6, PCSK6, HIPK2, C15orf48, | n/a |
| panel (275 | | TXNDC5, CPPED1, HLA.DPB1, PARD6G, | |
| unique genes) | | CYP4F11, FAM46C, IRX5, C3orf14, IGF1R, | |
| | | NGEF, SCCPDH, PPP2R2C, MUC16, ACOT2, | |
| | | SMPDL3B, C19orf51, MPV17L, SYK, CLEC2B, | |
| | | PTER, F2R, BST1, SYNM, EVL, CDKN1A, | |
| | | DOCK1, G0S2, MGST1, C12orf36, PLIN4, | |
| | | SERPINE2, JUB, SLC9A2, CLEC7A, TEKT2, | |
| | | EPPK1, OVCA2, MSMB, LGALS9C, MS4A8B, | |
| | | SDK1, PDLIM2, FOS, RPH3AL, KIF7, COL1A1, | |
| | | TEKT4P2, HLA.C, PAX3, SPRR2D, GIMAP6, | |
| | | SPRR2F, SPRR2C, DNER, ZDHHC2, GSTT1, | |
| | | ESR1, CDHR3, CYP2E1, TMEM190, BHLHE40, | |
| | | KRT13, KRT10, GNAL, RPSAP58, EPDR1, | |
| | | H2AFY2, GRTP1, NBPF1, SERPING1, PTAFR, | |
| | | KRT5, CHCHD10, HIC1, ZNF532, CROCCP2, | |
| | | HBEGF, ACAT2, S100A8, TFPI, C3, S100A7, | |
| | | HERC2P2, PLCL2, SORD, CD8A, MXRA8, | |
| | | IL2RB, NMU, LRRC26, BPIFA2, PRSS3, AK4, | |
| | | NPNT, SLC5A3, FCGBP, HTR3A, COL21A1, | |
| | | SLC5A5, MT2A, CYFIP2, XIST, ECM1, | |
| | | PTPRZ1, SLC5A8, MFSD6L, MLKL, ZC3H12A, | |
| | | ALDH1A3, SLC37A2, LOC286002, CCNJL, | |
| | | MEGF6, TM4SF1, SLC16A14, CXCR7, | |
| | | HOMER3, CYP1B1, ALDH3B2, SLC44A5, | |
| | | LTBP1, ANXA6, IL32, CDH26, MEX3D, VWA1, | |
| | | TPSAB1, HLA.DOA, ARRDC4, DMRTA2, | |
| | | SRRM3, IER3, RND1, REC8, RDH10, | |
| | | ATP6V0E2, POLR2J3, COL6A6, PCP4L1, | |
| | | GSTA1, RCBTB2, PYCR1, TSPAN8, B3GNT6, | |
| | | EIF1AX, CD177, PLXND1, IFITM1, DDIT4, | |
| | | KLHL29, KRT24, XYLT1, DLEC1, SERPINB3, | |
| | | IP6K3, TMEM220, LOC388796, KAL1, GNG4, | |
| | | C14orf167, CCNO, ERAP2, CCL5, TRIM31, | |
| | | RRAS, CLCA2, SLC29A1, SPRR1A, ARL4D, | |
| | | PTN, CPA3, OSM, TNS3, S100A13, IGF1, | |
| | | DPYSL3, SERPINB11, CDC25B, C11orf9, | |
| | | PMEPA1, HCAR2, SLC26A4, SHF, LOC90784, | |
| | | SCGB1A1, DNAI1, ABCG1, TMEM64, | |
| | | SEMA5A, CRYM, VAV2, NWD1, MAOB, | |
| | | ZNF117, GAS7, SPINK5, NCF2, DEFB1, KRT78, | |
| | | GPNMB, FABP5, MALAT1, MMP10, TP63, | |
| | | C7orf13, NLRP1, AKR1B15, GAS2L2, H1FX, | |
| | | CYP2B6, IL4I1, DUSP1, LYSMD2, PHYHD1, | |
| | | TOP1MT, SERPINF1, NFKBIZ, TPSB2, ZNF528, | |
| | | WDR96, BASP1, STEAP1, STEAP2, LPAR6, | |
| | | NCALD, OBSCN, MKI67, CPAMD8, D4S234E, | |
| | | SLC16A4, CRCT1, LY6D, RRM2, CIT, | |
| | | SC4MOL, NR1D1, ELF5, LPHN1, CRABP2, | |
| | | CEBPD, C6orf105, ANKRD9, C8orf4, | |
| | | TNFRSF18, TOR1B, TF, ACOT11, SLC35E2, | |
| | | LOC388564, SLC9A4, LRP12, ISYNA1, | |
| | | CARD11, MMP9, NCRNA00152, CROCC, CES1, | |
| | | TMX4, RAB3B, TNC, FOXN4, NCRNA00086, | |
| | | HLA.DQA1, RHCG, SLC9A3, SCGB3A1, SCD, | |
| | | ALOX15B, POU2F3, TCTEX1D4 | |

TABLE 5
Characteristics of the external asthma cohorts used in the validation of the asthma gene panel.

| | Asthmal²⁸ GEO GSE19187 | | | | | | Asthma2²⁹ GEO GSE46171* | | | | | |
|---|---|---|---|---|---|---|---|---|---|---|---|---|
| | Class | | | | | | | | | | | |
| | | | | | No | | | | | | No | |
| | Asthma | | | | Asthma | | Asthma | | | | Asthma | |
| | (n = 13) | | | | (n = 11) | | (n = 23) | | | | (n = 5) | |
| | Definition | | | | | | | | | | | |
| | Recurring | | | | | | | | | | | |
| | wheezing, | | | | No personal or | | | | | | | |
| | dyspnea, cough | | | | family | | | | | | No known | |
| | and bronchodilator | | | | history of atopy, | | History of | | | | airway | |
| | response | | | | rhinitis, or asthma | | asthma | | | | disease | |
| | Control | | | | | | | | | | | |
| | Controlled\hat{ } | | Uncontrolled | | n/a | | Controlled\hat{ } | | Uncontrolled | | n/a | |
| | Subjects | | | | | | | | | | | |
| | 7 | | 6 | | 11 | | 16 | | 7 | | 5 | |
| Age-years | 11.5 | (3.2) | 9.1 | (0.6) | 11.5 | (3.1) | 37 | (19-66)† | 29 | (25-46)† | 30 | (18-37)† |

| Female | 5 | (71.4%) | 2 | (33.3%) | 4 | (36.4%) | 36% | 20% | 14% |
|---|---|---|---|---|---|---|---|---|---|
| Race | | | | | | | | | |
| Caucasian | n/a | | n/a | | n/a | | 26% | 18% | 16% |
| African | n/a | | n/a | | n/a | | 8% | 2% | 0% |
| American | | | | | | | | | |
| Hispanic | n/a | | n/a | | n/a | | 6% | 0% | 0% |
| Other | n/a | | n/a | | n/a | | 6% | 2% | 2% |
| Rhinitis or | 7 | (100%) | 6 | (100%) | 0 | (0%) | 36% | 16% | 2% |
| atopic | | | | | | | | | |

| | | | | | | | | | | | |
|---|---|---|---|---|---|---|---|---|---|---|---|
| FEV1 | 97.6 | (13.2) | 78.2 | (7.7) | n/a | 97.8 | (16.5) | 91.2 | (10.8) | 98.3 | (11.0) |
| % predicted | | | | | | | | | | | |

| | | | | | | | | |
|---|---|---|---|---|---|---|---|---|
| FEV1/FVC | 89.3 | (5.6) | 76.5 | (3.2) | n/a | n/a | n/a | n/a |

| | | | | | | | | | |
|---|---|---|---|---|---|---|---|---|---|
| PC20 (mg/ml) | n/a | n/a | n/a | 4.5 | (5.1) | 4.4 | (5.2) | 28 | (27.1) |

Results are number (%) or mean (SD) unless otherwise indicated.
\hat{ }For Asthma1, criteria for control per NAEPP/EPR3 criteria. For Asthma2, criteria for control not specified.
*For Asthma2, data that the authors deposited in GEO GSE46171 are a subset of their published results.²⁹ GSE46171 has data for 16 of the 23 subjects with controlled asthma, 7 of the 11 subjects with uncontrolled asthma, and 5 of the 9 controls reported in the authors' publication.²⁹ The number of subjects with publically available data (GSE46171) that were used in these analyses are indicated. The summary statistics shown are drawn from the authors' publication on their reported sample.
†Median (range).

TABLE 6
Characteristics of the external cohorts with non-asthma respiratory conditions and controls used in the validation of the asthma gene panel.

| Allergic Rhinitis³⁵ | | URI Day 2²⁹ GEO | | URI Day 6²⁹ GEO | | Cystic Fibrosis³⁶ | | Smoking¹¹ | |
|---|---|---|---|---|---|---|---|---|---|
| GEO GSE43523* | | GSE46171\hat{ } | | GSE46171 | | GEO GSE40445 | | GEO GSE8987 | |
| Class | | | | | | | | | |
| Allergic | | | | | | Cystic | | | |
| Rhinitis | Control | URI | Control | URI | Control | Fibrosis | Control | Smoking | Control |
| N = 7 | N = 5 | N = 6 | N = 5 | N = 6 | N = 5 | N = 5 | N = 5 | N = 7 | N = 8 |

Defini-
tion**

| Age - | 37.9 (9.3) | 32.9 (7.8) | 30 (18-37)† | 30 (18-37)† | 30 (18-37)† | 30 (18-37)† | 14 | (4.2) | 14.8 | (1.1) | 47 | (12) | 43 | (18) |
|---|---|---|---|---|---|---|---|---|---|---|---|---|---|---|
| years | | | | | | | | | | | | | | |
| Female | 60% | 38.5% | 14% | 14% | 14% | 14% | 3 | (60%) | 2 | (40%) | 1 | (14.3%) | 2 | (25%) |
| Race | | | | | | | | | | | | | | |
| Cauca- | 0% | 0% | 16% | 16% | 16% | 16% | 5 | (100%) | 5 | (100%) | 3 | (42.9%) | 5 | (62.5%) |
| sian | | | | | | | | | | | | | | |

| | | | | | | | | | | | | |
|---|---|---|---|---|---|---|---|---|---|---|---|---|
| Af- | 0% | 0% | 0% | 0% | 0% | 0% | 0% | 0% | 2 | (28.6%) | 2 | (25%) |
| Amer- | | | | | | | | | | | | |
| ican | | | | | | | | | | | | |
| His- | 0% | 0% | 0% | 0% | 0% | 0% | 0% | 0% | 1 | (14.3%) | 1 | (12.5%) |
| panic | | | | | | | | | | | | |
| Other | 100% | 100% | 2% | 2% | 2% | 2% | 0% | 0% | 0 | (0%) | 0 | (0%) |

*Data that the authors deposited in GEO GSE43523 are a subset of their published results.³⁵ GSE43523 has data for 7 of the 15 subjects with allergic rhinitis, and 5 of the 13 controls reported in the authors' publication.³⁵ The number of subjects with publically available data (GSE43523) that were used in these analyses are indicated. The summary statistics shown are drawn from the authors' publication on their reported cohort.
\hat{ }Each subject provided a URI and control sample. The data that the authors deposited in GEO GSE46171 are a subset of their published results.²⁹ GSE46171 has data for 6 of the 9 healthy subjects reported in the authors' publication who provided samples during URI, and 5 of the 9 healthy subjects who provided samples after resolution of their URI.²⁹ The number of subjects with publically available data (GSE46171) that were used in these analyses are indicated. The summary statistics shown are drawn from the authors' publication on their reported cohort.
†Median (range).
**Definitions: Allergic Rhinitis = Rhinitis symptoms and ≥1 elevated sIgE to aeroallergen; Allergic rhinitis control = No symptoms, no sIgE to aeroallergen, total serum IgE < population mean. URI Day 2 = Day 2 following onset of “common cold” symptoms and no underlying airway disease; URI Day 2 control = No URI symptoms and no known airway disease. URI Day 6 = Day 6 following onset of “common cold” symptoms and no underlying airway disease; URI Day 6 control = No URI symptoms and no known airway disease. Cystic Fibrosis = Homozygous F508del mutation; Cystic Fibrosis control = Overweight but healthy. Smoking = ≥10 cigarettes/day in past month and smoking ≥10 pack years; Smoking control = Never smoker, no environmental cigarette exposure and no respiratory symptoms.

TABLE 7
Positive and negative predictive values (PPV and NPV respectively)
for the LR-RFE & Logistic asthma gene panel.

| Non-asthma data sets | PPV | NPV |
|---|---|---|
| Allergic Rhinitis | 0.00 (0.51) | 0.42 (0.16) |
| URI Day 2 | 0.50 (0.43) | 0.44 (0.22) |
| URI Day 6 | 0.00 (0.43) | 0.40 (0.23) |
| Cystic Fibrosis | 0.00 (0.44) | 0.50 (0.27) |
| Smoking | 0.00 (0.29) | 0.53 (0.36) |

Positive and negative predictive values (PPV and NPV respectively) obtained when the LR-RFE & Logistic asthma gene panel was applied to classifying samples in various microarray-derived data sets of subjects with non-asthma respiratory conditions and controls. Also shown in parentheses are the corresponding PPVs and NPVs obtained when random counterpart models are applied to these datasets for the same classification tasks.

### REFERENCES

1, Current Asthma Prevalence Percents by Age, Sex, and Race/Ethnicity, United States, 2012. Asthma Surveillance Data. National Health Interview Survey, National Center for Health Statistics, Centers for Disease Control and Prevention cdcgov/asthma/asthmadatahtm, downloaded 1/30/2017.
2. Yeatts K, Shy C, Sotir M, Music S, Herget C. Health consequences for children with undiagnosed asthma-like symptoms. Archives of pediatrics & adolescent medicine 157, 540-544 (2003).
3. Stempel D A, Spahn J D, Stanford R H, Rosenzweig J R, McLaughlin T P. The economic impact of children dispensed asthma medications without an asthma diagnosis. J Pediatr 148, 819-823 (2006).
4. Fanta C H. Asthma. N Engl J Med 360, 1002-1014 (2009).
5. Szefler S J, et al. Asthma outcomes: Biomarkers. Journal of Allergy and Clinical Immunology 129, S9-S23 (2012).
6. Reddel H K, et al. A summary of the new GINA strategy: a roadmap to asthma control. Eur Respir J 46, 622-639 (2015).
7. Expert Panel Report 3: Guidelines for the Diagnosis and Management of Asthma. (ed\hat{ }(eds). National Heart Lung and Blood Institute and National Asthma Education and Prevention Program (2007).
8. Gershon A S, Victor J C, Guan J, Aaron S D, To T. Pulmonary function testing in the diagnosis of asthma: a population study. Chest 141, 1190-1196 (2012).
9. Sokol K C, Sharma G, Lin Y L, Goldblum R M. Choosing wisely: adherence by physicians to recommended use of spirometry in the diagnosis and management of adult asthma. Am J Med 128, 502-508 (2015).
10. Petsky H L, et al. A systematic review and meta-analysis: tailoring asthma treatment on eosinophilic markers (exhaled nitric oxide or sputum eosinophils). Thorax 67, 199-208 (2012).
11. van Schayck C P, van Der Heijden F M, van Den Boom G, Tirimanna P R, van Herwaarden C L. Underdiagnosis of asthma: is the doctor or the patient to blame? The DIMCA project. Thorax 55, 562-565 (2000).
12. Sridhar S, et al. Smoking-induced gene expression changes in the bronchial airway are reflected in nasal and buccal epithelium. BMC Genomics 9, 259 (2008).
13. Wagener A H, et al. The impact of allergic rhinitis and asthma on human nasal and bronchial epithelial gene expression. PLoS One 8, e80257 (2013).
14. Guajardo J R, et al. Altered gene expression profiles in nasal respiratory epithelium reflect stable versus acute childhood asthma. J Allergy Clin Immunol 115, 243-251 (2005).
15. Poole A, et al. Dissecting childhood asthma with nasal transcriptomics distinguishes subphenotypes of disease. J Allergy Clin Immunol 133, 670-678 e612 (2014).
16. Byron S A, Van Keuren-Jensen K R, Engelthaler D M, Carpten J D, Craig D W. Translating RNA sequencing into clinical diagnostics: opportunities and challenges. Nat Rev Genet 17, 257-271 (2016).
17. Mendelsohn J. Personalizing oncology: perspectives and prospects. Journal of clinical oncology: official journal of the American Society of Clinical Oncology 31, 1904-1911 (2013).
18. Saeys Y, Inza I, Larranaga P. A review of feature selection techniques in bioinformatics. Bioinformatics 23, 2507-2517 (2007).
19. Witten I H, Frank E, Hall M A. Data mining: practical machine learning tools and techniques, 3rd edn. Morgan Kaufmann (2011).
20. Demsar J. Statistical Comparisons of Classifiers over Multiple Data Sets. J Mach Learn Res 7, 1-30 (2006).
21. The Childhood Asthma Management Program (CAMP): design, rationale, and methods. Childhood Asthma Management Program Research Group. Control Clin Trials 20, 91-120 (1999).
22. Covar R A, Fuhlbrigge A L, Williams P, Kelly H W, the Childhood Asthma Management Program Research G. The Childhood Asthma Management Program (CAMP): Contributions to the Understanding of Therapy and the Natural History of Childhood Asthma. Current respiratory care reports 1, 243-250 (2012).
23. Egan M, Bunyavanich S. Allergic rhinitis: the “Ghost Diagnosis” in patients with asthma. Asthma Research and Practice 1, DOI: 10.1186/s40733-40015-40008-40730 (2015).
24. Hoffman G E, Schadt E E. variancePartition: Quantifying and interpreting drivers of variation in complex gene expression studies. bioRxiv, doi: dx.doi.org/10.1101/040170 (2016).
25. Love M I, Huber W, Anders S. Moderated estimation of fold change and dispersion for RNA-seq data with DESeq2. Genome Biol 15, 550 (2014).
26. Subramanian A, et al. Gene set enrichment analysis: a knowledge-based approach for interpreting genome-wide expression profiles. Proc Natl Acad Sci USA 102, 15545-15550 (2005).
27. Whalen S, Pandey O P, Pandey G. Predicting protein function and other biomedical characteristics with heterogeneous ensembles. Methods 93, 92-102 (2016).
28. Powers D M. Evaluation: from precision, recall and F-measure to ROC, informedness, markedness and correlation. (2011).
29. Mathias R A. Introduction to genetics and genomics in asthma: genetics of asthma. Advances in experimental medicine and biology 795, 125-155 (2014).
30. Giovannini-Chami L, et al. Distinct epithelial gene expression phenotypes in childhood respiratory allergy. Eur Respir J 39, 1197-1205 (2012).
31. McErlean P, et al. Asthmatics with exacerbation during acute respiratory illness exhibit unique transcriptional signatures within the nasal mucosa. Genome medicine 6, 1 (2014).
32. Zhang W, et al. Comparison of RNA-seq and microarray-based models for clinical endpoint prediction. Genome Biol 16, 133 (2015).
33. Su Z, et al. An investigation of biomarkers derived from legacy microarray data for their utility in the RNA-seq era. Genome Biol 15, 523 (2014).
34. Venet D, Dumont J E, Detours V. Most Random Gene Expression Signatures Are Significantly Associated with Breast Cancer Outcome. PLoS computational biology 7, e1002240 (2011).
35. Chibon F. Cancer gene expression signatures—the rise and fall? European journal of cancer 49, 2000-2009 (2013).
36. Imoto Y, et al. Cystatin S N upregulation in patients with seasonal allergic rhinitis. PLoS One 8, e67057 (2013).
37. Clarke L A, Sousa L, Barreto C, Amaral M D. Changes in transcriptome of native nasal epithelium expressing F508del-CFTR and intersecting data from comparable studies. Respir Res 14, 38 (2013).
38. Oliver B G, Robinson P, Peters M, Black J. Viral infections and asthma: an inflammatory interface? Eur Respir J 44, 1666-1681 (2014).
39. Scott S, Currie J, Albert P, Calverley P, Wilding J P. Risk of misdiagnosis, health-related quality of life, and BMI in patients who are overweight with doctor-diagnosed asthma. Chest 141, 616-624 (2012).
40. Kulkarni M M. Digital multiplexed gene expression analysis using the NanoString nCounter system. Current protocols in molecular biology/edited by Frederick M Ausubel [et al] Chapter 25, Unit25B 10 (2011).
41. Veldman-Jones M H, et al. Evaluating Robustness and Sensitivity of the NanoString Technologies nCounter Platform to Enable Multiplexed Gene Expression Analysis of Clinical Samples. Cancer research 75, 2587-2593 (2015).
42. Leong H S, et al. Efficient molecular subtype classification of high-grade serous ovarian cancer. The Journal of pathology 236, 272-277 (2015).
43. Cardoso F, et al. 70-Gene Signature as an Aid to Treatment Decisions in Early-Stage Breast Cancer. N Engl J Med 375, 717-729 (2016).
44. Paik S, et al. A multigene assay to predict recurrence of tamoxifen-treated, nodenegative breast cancer. N Engl J Med 351, 2817-2826 (2004).
45. Wechsler M E. Managing asthma in primary care: putting new guideline recommendations into context. Mayo Clin Proc 84, 707-717 (2009).
46. Physician Fee Schedule Search. Centers for Medicare & Medicaid Services, available athttps://wwwcmsgov/apps/physician-fee-schedule/search/search-criteriaaspx and accessed on Jan. 30, 2017, (2016).
47. Goodwin S, McPherson J D, McCombie W R. Coming of age: ten years of nextgeneration sequencing technologies. Nat Rev Genet 17, 333-351 (2016).
48. Asthma in the U S. Centers for Disease Control and Prevention Vitalsigns http://wwwcdcgov/vitalsigns/asthma/, downloaded Jan. 30, 2017, (2011).
49. Cowling B J, et al. Comparative epidemiology of pandemic and seasonal influenza A in households. N Engl J Med 362, 2175-2184 (2010).
50. Bunyavanich S, Schadt E E. Systems biology of asthma and allergic diseases: A multiscale approach. J Allergy Clin Immunol, (2014).
51. Sordillo J, Raby B A. Gene expression profiling in asthma. Advances in experimental medicine and biology 795, 157-181 (2014).
52. Jain V V, Allison D R, Andrews S, Mejia J, Mills P K, Peterson M W. Misdiagnosis Among Frequent Exacerbators of Clinically Diagnosed Asthma and COPD in Absence of Confirmation of Airflow Obstruction. Lung 193, 505-512 (2015).
53. Brower V. Biomarkers: Portents of malignancy. Nature 471, S19-21 (2011).
54. Muraro A, et al. Precision medicine in patients with allergic diseases: Airway diseases and atopic dermatitis-PRACTALL document of the European Academy of Allergy and Clinical Immunology and the American Academy of Allergy, Asthma & Immunology. J Allergy Clin Immunol 137, 1347-1358 (2016).
55. Himes B E, et al. Genome-wide association analysis identifies PDE4D as an asthma susceptibility gene. Am J Hum Genet 84, 581-593 (2009).
56. Fromer M, et al. Gene expression elucidates functional impact of polygenic risk for schizophrenia. Nat Neurosci, (2016).
57. Langmead B, Trapnell C, Pop M, Salzberg S L. Ultrafast and memory-efficient alignment of short DNA sequences to the human genome. Genome Biol 10, R25 (2009).
58. Trapnell C, Pachter L, Salzberg S L. TopHat: discovering splice junctions with RNA-Seq. Bioinformatics 25, 1105-1111 (2009).
59. Trapnell C, et al. Transcript assembly and quantification by RNA-Seq reveals unannotated transcripts and isoform switching during cell differentiation. Nat Biotechnol 28, 511-515 (2010).
60. DeLuca D S, et al. RNA-SeQC: RNA-seq metrics for quality control and process optimization. Bioinformatics 28, 1530-1532 (2012).
61. Pedregosa F, Varoquaux Ge, Gramfort A, Michel V, Thirion B, others. Scikit-learn: Machine Learning in Python. Journal of Machine Learning Research 12, 2825-2830 (2011).
62. Guyon I, Weston, J, Barnhill, S, Vapnik, V. Gene selection for cancer classification using support vector machines. Machine Learning 46, 389-422 (2002).
63. Schadt E E, Friend S H, Shaywitz D A. A network view of disease and compound screening. Nature reviews Drug discovery 8, 286-295 (2009).
64. Bewick V, Cheek L, Ball J. Statistics review 14: Logistic regression. Crit Care 9, 112-118 (2005).
65. Burges C J. A tutorial on support vector machines for pattern recognition. Data mining and knowledge discovery 2, 121-167 (1998).
66. Freund Y, Schapire R E. A Decision-Theoretic Generalization of On-Line Learning and an Application to Boosting. J Comput Syst Sci 55, 119-139 (1997).
67. Breiman L. Random Forests. Machine Learning 45, 5-32 (2001).
68. Hollander M, Wolfe D A, Chicken E. Nonparametric statistical methods. John Wiley & Sons (2013).
69. Vidaurre D, Bielza C, Larrafiaga P. A Survey of L1 Regression. International Statistical Review 81, 361-387 (2013).
70. Barrett T, et al. NCBI GEO: archive for functional genomics data sets—update. Nucleic Acids Res 41, D991-995 (2013).

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed.

All patents, applications, publications, test methods, literature, and other materials cited herein are hereby incorporated by reference in their entirety as if physically present in this specification.

1. A method for diagnosing asthma in a subject, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

2. A method for detection of asthma in a subject, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

3. A method for differentially diagnosing asthma from other respiratory disorders in a subject, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

4. A method for classifying a subject as having asthma or not having asthma, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

5. A method for monitoring asthma in a subject, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

6. A method for selecting a subject for a clinical trial for asthma therapeutic compositions and/or methods, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold; and
d) identifying the subject as (i) having asthma when the probability output is greater than or equal to the optimal classification threshold or (ii) not having asthma when the probability output is less than the optimal classification threshold.

7. A method for treating asthma in a subject, comprising the steps of:
a) measuring the gene expression profile(s) of at least one of the genes in the asthma gene panel in a nasal swab/scraping/brushing/wash/sponge collected from the subject;
b) performing classification analysis on the gene counts obtained from the gene expression profile(s);
c) comparing the probability output obtained from the classification analysis to the optimal classification threshold;
d) identifying the subject as (i) having asthma when the probability output is greater than or equal to the optimal classification threshold or (ii) not having asthma when the probability output is less than the optimal classification threshold; and
e) utilizing appropriate therapeutic compositions and/or methods if the subject has asthma.

8. The method as described in claim 1, wherein step (a) further comprises the steps of (i) brushing/swabbing/scraping/washing/sponging the patient's nose, (ii) obtaining and appropriately preserving the nasal brushing/swab/scraping/wash/sponge sample, and (iii) assaying the gene expression profile of the cells and tissue contained in the sample, whether by isolating RNA as described herein or by use of a RNA profiling system that does not require a separate isolation step.

9. The method as described in claim 1, wherein the classification analysis comprises Logistic Regression-Recursive Feature Elimination (LR-RFE) algorithms in combination with Logistic algorithm, the asthma gene panel consists of the LR-RFE & Logistic asthma gene panel, and the optimal classification threshold is about 0.76.

10. The method as described in claim 1, wherein the classification analysis comprises LR-RFE algorithm in combination with SVM-Linear algorithms, the asthma gene panel consists of the LR-RFE & SVM-Linear asthma gene panel, and the optimal classification threshold is about 0.52.

11. The method as described in claim 1, wherein the classification analysis comprises the SVM-RFE algorithm in combination with the SVM-Linear algorithms, the asthma gene panel consists of the SVM-RFE & SVM-Linear asthma gene panel, and the optimal classification threshold is about 0.64.

12. The method as described in claim 1, wherein the classification analysis comprises the SVM-RFE algorithm in combination with the Logistic algorithms, the asthma gene panel consists of the SVM-RFE & Logistic asthma gene panel, and the optimal classification threshold is about 0.69.

13. The method as described in claim 1, wherein the classification analysis comprises the LR-RFE algorithm in combination with the AdaBoost algorithms, the asthma gene panel consists of the LR-RFE & AdaBoost asthma gene panel, and the optimal classification threshold is about 0.49.

14. The method as described in claim 1, wherein the classification analysis comprises the LR-RFE algorithm in combination with the RandomForest algorithms, the asthma gene panel consists of the LR-RFE & RandomForest asthma gene panel, and the optimal classification threshold is about 0.60.

15. The method as described in claim 1, wherein the classification analysis comprises the SVM-RFE algorithm in combination with the RandomForest algorithms, the asthma gene panel consists of the SVM-RFE & RandomForest asthma gene panel, and the optimal classification threshold is about 0.50.

16. The method as described in claim 1, wherein the classification analysis comprises the SVM-RFE algorithm in combination with the AdaBoost algorithm, the asthma gene panel consists of the SVM-RFE & AdaBoost asthma gene panel, and the optimal classification threshold is about 0.55.

17. The method as described in claim 1, wherein steps (b) and/or (c) and/or (d) are performed by a computer.

18. A kit for diagnosing and/or detecting asthma in a subject, said kit comprising probes directed towards one or more of the genes in the asthma gene panel, wherein the probes can be used to determine the expression levels of one or more of the genes in the asthma gene panel.

19. The kit of claim 12, further comprising: a detection means; an amplification means; and control probes.
